(19) United States
(12) Patent Application Publication
(10) Pub. No.: US 2024/0109972 A1
(43) Pub. Date: Apr. 4, 2024

(54) ANTIBODY AND TAXANE COMBINATION THERAPY

(71) Applicants: Genmab A/S, Copenhagen V (DK); BioNTech SE, Mainz (DE)

(72) Inventors: Ugur SAHIN, Mainz (DE); Alexander MUIK, Mainz (DE); Ulf FORSSMANN, Copenhagen V (DK); Maria JURE-KUNKEL, Plainsboro, NJ (US); Manish GUPTA, Plainsboro, NJ (US); Tahamtan AHMADI, Plainsboro, NJ (US); Kathy AMIRI, Plainsboro, NJ (US); Gaurav BAJAJ, Plainsboro, NJ (US)

(21) Appl. No.: 18/038,818

(22) PCT Filed: Dec. 7, 2021
(86) PCT No.: PCT/EP2021/084659

§ 371 Date: May 25, 2023

Related U.S. Application Data
(60) Provisional application No. 63/122,042, filed on Dec. 7, 2020.

(51) Int. Cl. C07K 16/28; A61K 31/337; A61K 45/06; A61P 35/00

(52) U.S. Cl. CPC C07K 16/2878; A61K 31/337; A61K 45/06; A61P 35/00; C07K 16/2827; A61K 2039/505

(57) ABSTRACT

The present invention provides combination therapy to reduce or prevent progression of a tumor or treating cancer in a subject. The combination therapy comprises administration of a binding agent that binds to human CD137 and to human PD-L1; and a taxane chemotherapeutic agent.

### RELATED APPLICATIONS

This application is a 35 U.S.C. § 371 filing of International Patent Application No. PCT/EP2021/084659, filed Dec. 7, 2021, which claims priority to U.S. Provisional Patent Application Ser. No. 63/122,042, filed Dec. 7, 2020, the entire disclosures of which are hereby incorporated herein by reference.

### SEQUENCE LISTING

The instant application contains a Sequence Listing which has been submitted electronically in ASCII format and is hereby incorporated by reference in its entirety. Said ASCII file, created on May 23, 2023, is named 743548_GMB9-008US_ST25.txt and is 59,128 bytes in size.

### FIELD OF INVENTION

The present invention relates to combination therapy using a binding agent that binds to human CD137 and to human PD-L1 in combination with a taxane chemotherapeutic agent to reduce or prevent progression of a tumor or treating cancer.

### BACKGROUND OF THE INVENTION

CD137 (4-1BB, TNFRSF9) is a member of the tumor necrosis factor (TNF) receptor (TNFR) family. CD137 is a co-stimulatory molecule on CD8+ and CD4+ T cells, regulatory T cells (Tregs), natural killer (NK) and NKT cells, B cells and neutrophils. On T cells, CD137 is not constitutively expressed, but induced upon T-cell receptor (TCR)-activation. Stimulation via its natural ligand 4-1BBL or agonist antibodies leads to signaling using TNFR-associated factor (TRAF)-2 and TRAF-1 as adaptors. Early signaling by CD137 involves K-63 poly-ubiquitination reactions that ultimately result in activation of the nuclear factor (NF)-κB and mitogen-activated protein (MAP)-kinase pathways. Signaling leads to increased T cell co-stimulation, proliferation, cytokine production, maturation and prolonged CD8⁺ T-cell survival. Agonistic antibodies against CD137 have been shown to promote anti-tumor control by T cells in various pre-clinical models (Murillo et al. 2008 Clin. Cancer Res. 14(21): 6895-6906). Antibodies stimulating CD137 can induce survival and proliferation of T cells, thereby enhancing the anti-tumor immune response. Antibodies stimulating CD137 have been disclosed in the prior art, and include urelumab, a human IgG4 antibody (WO2005035584) and utomilumab, a human IgG2 antibody (Fisher et al. 2012 Cancer Immunol. Immunother. 61: 1721-1733).

Programmed death ligand 1 (PD-L1, PDL1, CD274, B7H1) is a 33 kDa, single-pass type I membrane protein. Three isoforms of PD-L1 have been described, based on alternative splicing. PD-L1 belongs to the immunoglobulin (Ig) superfamily and contains one Ig-like C2-type domain and one Ig-like V-type domain. Freshly isolated T and B cells express negligible amounts of PD-L1 and a fraction (about 16%) of CD14⁺ monocytes constitutively express PD-L1. However, interferon-γ (IFNγ) is known to upregulate PD-L1 on tumor cells.

PD-L1 obstructs anti-tumor immunity by 1) tolerizing tumor-reactive T cells by binding to its receptor, programmed cell death protein 1 (PD-1) (CD279) on activated T cells; 2) rendering tumor cells resistant to CD8⁺ T cell and Fas ligand—mediated lysis by PD-1 signaling through tumor cell-expressed PD-L1; 3) tolerizing T cells by reverse signaling through T cell—expressed CD80 (B7.1); and 4) promoting the development and maintenance of induced T regulatory cells. PD-L1 is expressed in many human cancers, including melanoma, ovarian, lung and colon cancer (Latchman et al., 2004 Proc Natl Acad Sci USA 101, 10691-6).

PD-L1 blocking antibodies have shown clinical activity in several cancers known to overexpress PD-L1 (incl. melanoma, NSCLC). For example, atezolizumab is a humanized IgG1 monoclonal antibody against PD-L1. It is currently in clinical trials as an immunotherapy for several indications including various types of solid tumors (see e.g. Rittmeyer et al., 2017 Lancet 389:255-265) and is approved for non-small-cell lung cancer and bladder cancer indications. Avelumab, a PD-L1 antibody, (Kaufman et al Lancet Oncol. 201647(10):1374-1385) has been approved by the FDA for the treatment of adults and pediatric patients 12 years and older with metastatic Merkel cell carcinoma, and is currently in clinical trials in several cancer indiciations, including bladder cancer, gastric cancer, head and neck cancer, mesothelioma, NSCLC, ovarian cancer and renal cancer. Durvalumab, a PD-L1 antibody, is approved for locally advanced or metastatic urothelial carcinoma indications, and is in clinical development in multiple solid tumors and blood cancers (see e.g. Massard et al., 2016 J Clin Oncol. 34(26):3119-25). Further anti-PD-L1 antibodies have been described e.g. in WO2004004771.

Horton et al. (J Immunother Cancer. 2015; 3(Suppl 2): O10) discloses combination of an agonistic 4-1BB antibody with a neutralizing PD-L1 antibody. WO 2019/025545 provides binding agents, such as bispecific antibodies, binding human PD-L1 and binding human CD137.

However, despite these advances in the art there is a considerable need for improved therapies targeting PD-L1 and CD137.

### SUMMARY OF THE INVENTION

It is an object of the present invention to provide a method for reducing or preventing progression of a tumor or treating cancer in a subject, comprising providing to the subject combined treatment with
i) a binding agent comprising a first binding region binding to human CD137, and a second binding region binding to human PD-L1; and
ii) a taxane chemotherapeutic agent.

The amount of binding agent administered in each dose and/or in each treatment cycle may be
a) about 0.3-5 mg/kg body weight or about 25-400 mg in total; and/or
b) about 2.1×10⁻⁹−3.4×10⁻⁸ mol/kg body weight or about 1.7×10⁻⁷-2.7×10⁻⁶ mol in total.

It is a further object of the invention to provide a binding agent comprising a first binding region binding to human CD137, and a second binding region binding to human PD-L1 for use in treatment of cancer or for use in reducing or preventing progression of a tumor, wherein the binding agent is used in combination with a taxane chemotherapeutic agent.

Another aspect of the invention provides a taxane chemotherapeutic agent for use in treatment of cancer or for use in reducing or preventing progression of a tumor, wherein the taxane chemotherapeutic agent is used in combination with a binding agent comprising a first binding region binding to human CD137, and a second binding region binding to human PD-L1.

Finally, it is an object of the invention to provide a composition comprising a taxane chemotherapeutic agent and a binding agent comprising a first binding region binding to human CD137, and a second binding region binding to human PD-L1.

### BRIEF DESCRIPTION OF THE DRAWINGS

FIG. 1: Schematic representation of the anticipated mode of action of CD137×PD-L1 bispecific antibodies. (A) PD-L1 is expressed on antigen-presenting cells (APCs) as well as on tumor cells. PD-L1 binding to T cells expressing the negative regulatory molecule PD-1 effectively overrides T cell activation signals and eventually leads to T cell inhibition. (B) Upon addition of a CD137×PD-L1 bispecific antibody, the inhibitory PD-1:PD-L1 interaction is blocked via the PD-L1-specific arm and at the same time, the bispecific antibody, through the cell-cell interaction provides agonistic signaling to CD137 expressed on the T cells resulting in strong T cell co-stimulation

FIG. 2: Schematic outline of clinical trial design.

FIG. 3: Dose escalation; best percent change from baseline in tumor size, all patients. Data cut-off: Sep. 29, 2020. Post-baseline scans were not conducted for five patients. ^{a}Minimum duration of response (5 weeks) per RECIST v1.1 not reached. ^{b}PR was not confirmed on a subsequent scan. NE, non-evaluable; NSCLC, non-small cell lung cancer; PD, progressive disease; PD-(L)1, programmed death (ligand) 1; PR, partial response; SD, stable disease; SoD, sum of diameters; uPR, unconfirmed partial response.

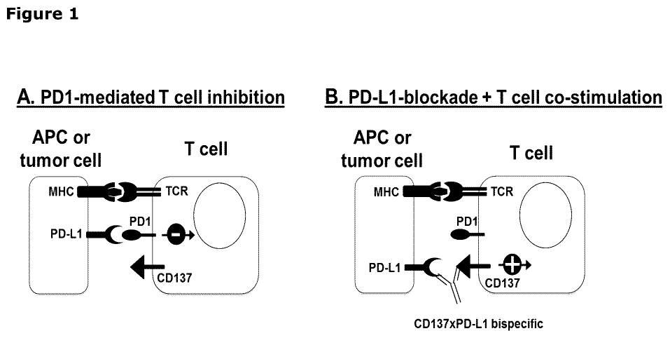

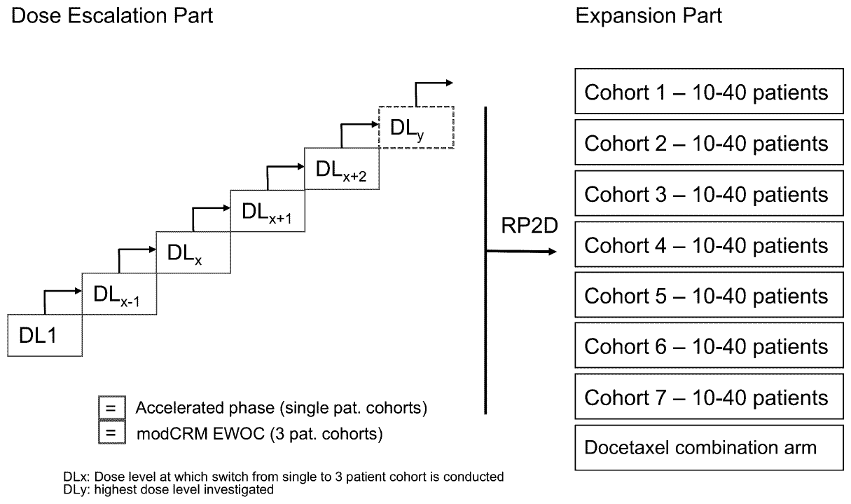

FIG. 4: Dose escalation; Best change from baseline in tumor size, patients with NSCLC. Data cut-off: Sep. 29, 2020.
^{a}PR was not confirmed by a subsequent scan.
bPD-L1 expression was assessed in archival tumor specimens.
BOR, best overall response; CR, complete response; ICI, immune checkpoint inhibitor; NA, not available; PD, progressive disease; PD-(L)1, programmed death (ligand) 1; PR, partial response; RECIST, Response Evaluation Criteria in Solid Tumors; SD, stable disease; SoD, sum of diameters; TPS, tumor proportion score; uPR, unconfirmed partial response.

FIG. 5: Expansion cohort 1; A) Best change from baseline in tumor size, B) Target lesion SoD change from baseline. Data cut-off: Oct. 12, 2020.
Denotes patients with ongoing treatment.
^{a}PR was not confirmed by a subsequent scan. ^{b}PD-L1 expression was assessed in tumor biopsies obtained prior to initiation of GEN1046 treatment (22C3 pharmDx assay, HistoGeneX, Belgium). Includes all patients who had at least one post-baseline tumor assessment (schedule is every 6 weeks), and thus could be assessed for clinical benefit; 6 of 12 patients are still on treatment. Of the remaining 12 patients not shown, three patients had clinical progression prior to first response assessment, and nine patients were still receiving treatment and had not had a first response assessment.
BOR and time point response assessed using RECIST 1.1; NA: Assessment succeeding first PD. BOR, best overall response; ICI, immune checkpoint inhibitor; NA, not available; NE, non-evaluable; NSCLC, non-small cell lung cancer; PD, progressive disease; PD-(L)1, programmed death (ligand) 1; PR, partial response; RECIST, Response Evaluation Criteria in Solid Tumors; SD, stable disease; SoD, sum of diameters; TPS, tumor proportion score; uPR, unconfirmed partial response.

FIG. 6: Model Predicted Maximal Trimer Formation and Receptor Occupancy for PD-L1 at 100 mg dose administered once every third week (1Q3W).

FIG. 7: MC38 syngeneic tumor model established by subcutaneous inoculation of 1×10⁶ MC38 cells into C57BL/6 mice. When tumors reached an average volume of 64 mm³, mice were randomized and treated with mbsIgG2a-PD-L1×4-1BB (0.5 mg/kg; 2QW×3), docetaxel (10 mg/kg; QW×3), either alone or in combination, or PBS. A. Data shown are the median tumor volume per treatment group (n=10) with data carried forward for animals that reached termination criteria. Growth curves were discontinued when <50% of the animals within a treatment group remained alive. Arrows indicate days of treatment. B. Progression-free survival, defined as the percentage of mice with tumor volume smaller than 500 mm³, is shown as Kaplan Meier curve. Mantel Cox analysis was used to compare survival between treatment groups after progression of all animals to a tumor volume above 500 mm³ (Table 14).

### DETAILED DESCRIPTION OF THE INVENTION

### Definitions

The term “binding agent” in the context of the present invention refers to any agent capable of binding to desired antigens. In certain embodiments of the invention, the binding agent is an antibody, antibody fragment, or construct thereof. The binding agent may also comprise synthetic, modified or non-naturally occurring moieties, in particular non-peptide moieties. Such moieties may, for example, link desired antigen-binding functionalities or regions such as antibodies or antibody fragments. In one embodiment, the binding agent is a synthetic construct comprising antigen-binding CDRs or variable regions.

The term “immunoglobulin” refers to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized. See for instance Fundamental Immunology Ch. 7 (Paul, W., ed., 2nd ed. Raven Press, N.Y. (1989)). Briefly, each heavy chain typically is comprised of a heavy chain variable region (abbreviated herein as V_{H} or VH) and a heavy chain constant region (abbreviated herein as C_{H} or CH). The heavy chain constant region typically is comprised of three domains, CH1, CH2, and CH3. The hinge region is the region between the CH1 and CH2 domains of the heavy chain and is highly flexible. Disulphide bonds in the hinge region are part of the interactions between two heavy chains in an IgG molecule. Each light chain typically is comprised of a light chain variable region (abbreviated herein as V_{L} or VL) and a light chain constant region (abbreviated herein as C_{L} or CL). The light chain constant region typically is comprised of one domain, CL. The VH and VL regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each VH and VL is typically composed of three CDRs and four FRs, arranged from amino-terminus to carboxy-terminus in the following order: FR1, CDR1, FR2, CDR2, FR3, CDR3, FR4 (see also Chothia and Lesk J. Mol. Biol. 196 901-917 (1987)). Unless otherwise stated or contradicted by context, CDR sequences herein are identified according to IMGT rules using DomainGapAlign (Lefranc MP., Nucleic Acids Research 1999; 27:209-212 and Ehrenmann F., Kaas Q. and Lefranc M.-P. Nucleic Acids Res., 38, D301-307 (2010); see also internet http address www.imgt.org/). Unless otherwise stated or contradicted by context, reference to amino acid positions in the constant regions in the present invention is according to the EU-numbering (Edelman et al., Proc Natl Acad Sci USA. 1969 May; 63(1):78-85; Kabat et al., Sequences of Proteins of Immunological Interest, Fifth Edition. 1991 NIH Publication No. 91-3242).

The term “amino acid” and “amino acid residue” may herein be used interchangeably, and are not to be understood limiting. Amino acids are organic compounds containing amine (—NH₂) and carboxyl (—COOH) functional groups, along with a side chain (R group) specific to each amino acid. In the context of the present invention, amino acids may be classified based on structure and chemical characteristics. Thus, classes of amino acids may be reflected in one or both of the following tables:

TABLE 1
Main classification based on structure and general
chemical characterization of R group

| Class | Amino acid |
|---|---|
| Acidic Residues | D and E |
| Basic Residues | K, R, and H |
| Hydrophilic Uncharged Residues | S, T, N, and Q |
| Aliphatic Uncharged Residues | G, A, V, L, and I |
| Non-polar Uncharged Residues | C, M, and P |
| Aromatic Residues | F, Y, and W |

TABLE 2
Alternative Physical and Functional Classifications
of Amino Acid Residues

| Class | Amino acid |
|---|---|
| Hydroxyl group containing residues | S and T |
| Aliphatic residues | I, L, V, and M |
| Cycloalkenyl-associated residues | F, H, W, and Y |
| Hydrophobic residues | A, C, F, G, H, I, L, |
| | M, R, T, V, W, and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in turn formation | A, C, D, E, G, H, K, |
| | N, Q, R, S, P, and T |
| Flexible residues | Q, T, K, S, G, P, D, E, and R |

Substitution of one amino acid for another may be classified as a conservative or non-conservative substitution. In the context of the invention, a “conservative substitution” is a substitution of one amino acid with another amino acid having similar structural and/or chemical characteristics, such substitution of one amino acid residue for another amino acid residue of the same class as defined in any of the two tables above: for example, leucine may be substituted with isoleucine as thay are both aliphatic, branched hydrophobes. Similarly, aspartic acid may be substituted with glutamic acid since they are both small, negatively charged residues.

The term “amino acid corresponding to position . . . ” as used herein refers to an amino acid position number in a human IgG1 heavy chain. Corresponding amino acid positions in other immunoglobulins may be found by alignment with human IgG1. Thus, an amino acid or segment in one sequence that “corresponds to” an amino acid or segment in another sequence is one that aligns with the other amino acid or segment using a standard sequence alignment program such as ALIGN, ClustalW or similar, typically at default settings and has at least 50%, at least 80%, at least 90%, or at least 95% identity to a human IgG1 heavy chain. It is considered well-known in the art how to align a sequence or segment in a sequence and thereby determine the corresponding position in a sequence to an amino acid position according to the present invention.

The term “antibody” (Ab) in the context of the present invention refers to an immunoglobulin molecule, a fragment of an immunoglobulin molecule, or a derivative of either thereof, which has the ability to specifically bind to an antigen under typical physiological conditions with a half-life of significant periods of time, such as at least about 30 minutes, at least about 45 minutes, at least about one hour, at least about two hours, at least about four hours, at least about 8 hours, at least about 12 hours, about 24 hours or more, about 48 hours or more, about 3, 4, 5, 6, 7 or more days, etc., or any other relevant functionally-defined period (such as a time sufficient to induce, promote, enhance, and/or modulate a physiological response associated with antibody binding to the antigen and/or time sufficient for the antibody to recruit an effector activity). The variable regions of the heavy and light chains of the immunoglobulin molecule contain a binding domain that interacts with an antigen. The term “antigen-binding region”, wherein used herein, refers to the region which interacts with the antigen and comprises both a VH region and a VL region. The term antibody when used herein comprises not only monospecific antibodies, but also multispecific antibodies which comprise multiple, such as two or more, e.g. three or more, different antigen-binding regions. The constant regions of the antibodies (Abs) may mediate the binding of the immunoglobulin to host tissues or factors, including various cells of the immune system (such as effector cells) and components of the complement system such as C1q, the first component in the classical pathway of complement activation. As indicated above, the term antibody herein, unless otherwise stated or clearly contradicted by context, includes fragments of an antibody that are antigen-binding fragments, i.e., retain the ability to specifically bind to the antigen. It has been shown that the antigen-binding function of an antibody may be performed by fragments of a full-length antibody. Examples of antigen-binding fragments encompassed within the term “antibody” include (i) a Fab′ or Fab fragment, a monovalent fragment consisting of the VL, VH, CL and CH1 domains, or a monovalent antibody as described in WO2007059782 (Genmab); (ii) F(ab′)₂ fragments, bivalent fragments comprising two Fab fragments linked by a disulfide bridge at the hinge region; (iii) a Fd fragment consisting essentially of the VH and CH1 domains; (iv) a Fv fragment consisting essentially of the VL and VH domains of a single arm of an antibody, (v) a dAb fragment (Ward et al., Nature 341, 544-546 (1989)), which consists essentially of a VH domain and also called domain antibodies (Holt et al; Trends Biotechnol. 2003 November;21(11):484-90); (vi) camelid or Nanobody molecules (Revets et al; Expert Opin Biol Ther. 2005 January;5(1):111-24) and (vii) an isolated complementarity determining region (CDR). Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (known as single chain antibodies or single chain Fv (scFv), see for instance Bird et al., Science 242, 423-426 (1988) and Huston et al., PNAS USA 85, 5879-5883 (1988)). Such single chain antibodies are encompassed within the term antibody unless otherwise noted or clearly indicated by context. Although such fragments are generally included within the meaning of antibody, they collectively and each independently are unique features of the present invention, exhibiting different biological properties and utility. These and other useful antibody fragments in the context of the present invention, as well as bispecific formats of such fragments, are discussed further herein. It also should be understood that the term antibody, unless specified otherwise, also includes polyclonal antibodies, monoclonal antibodies (mAbs), antibody-like polypeptides, such as chimeric antibodies and humanized antibodies, and antibody fragments retaining the ability to specifically bind to the antigen (antigen-binding fragments) provided by any known technique, such as enzymatic cleavage, peptide synthesis, and recombinant techniques. An antibody as generated can possess any isotype. As used herein, the term “isotype” refers to the immunoglobulin class (for instance IgG1, IgG2, IgG3, IgG4, IgD, IgA, IgE, or IgM) that is encoded by heavy chain constant region genes. When a particular isotype, e.g. IgG1, is mentioned herein, the term is not limited to a specific isotype sequence, e.g. a particular IgG1 sequence, but is used to indicate that the antibody is closer in sequence to that isotype, e.g. IgG1, than to other isotypes. Thus, e.g. an IgG1 antibody of the invention may be a sequence variant of a naturally occurring IgG1 antibody, including variations in the constant regions.

The term “bispecific antibody” or “bs” in the context of the present invention refers to an antibody having two different antigen-binding regions defined by different antibody sequences. In some embodiments, said different antigen-binding regions bind different epitopes on the same antigen. However, in preferred embodiments, said different antigen-binding regions bind different target antigens. A bispecific antibody can be of any format, including any of the bispecific antibody formats described herein below.

The term “full-length” when used in the context of an antibody indicates that the antibody is not a fragment, but contains all of the domains of the particular isotype normally found for that isotype in nature, e.g. the VH, CH1, CH2, CH3, hinge, VL and CL domains for an IgG1 antibody. In particular embodiments, a full-length antibody contains two heavy and two light chains. Each chain contains constant (C) and variable (V) regions, which can be divided into domains designated CHI, CH2, CH3, VH for the heavy chain, and CL, VL for the light chain. Preferably, the domains of the heavy chains are arranged in the order of a natural antibody: VH-CH1-CH2-CH3; which means that the VH domain is adjacent to the CHI domain, followed by a CH2 domain and subsequently followed by a CH3 domain. Preferably, the domains of the light chains are also present in the order of a natural antibody: VL-CL; meaning that the VL domain is adjacent to the CL domain.

The term “human antibody”, as used herein, is intended to include antibodies having variable and framework regions derived from human germline immunoglobulin sequences and a human immunoglobulin constant domain. The human antibodies of the invention may include amino acid residues not encoded by human germline immunoglobulin sequences (e.g., mutations, insertions or deletions introduced by random or site-specific mutagenesis in vitro or by somatic mutation in vivo). However, the term “human antibody”, as used herein, is not intended to include antibodies in which CDR sequences derived from the germline of another non-human species, such as a mouse, have been grafted onto human framework sequences.

The term “humanized antibody” as used herein, refers to a genetically engineered non-human antibody, which contains human antibody constant domains and non-human variable domains modified to contain a high level of sequence homology to human variable domains. This can be achieved by grafting of the six non-human antibody complementarity-determining regions (CDRs), which together form the antigen binding site, onto a homologous human acceptor framework region (FR) (see WO92/22653 and EP0629240). In order to fully reconstitute the binding affinity and specificity of the parental antibody, the substitution of framework residues from the parental antibody (i.e. the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions. Optionally, additional amino acid modifications, which are not necessarily back-mutations, may be applied to obtain a humanized antibody with preferred characteristics, such as affinity and biochemical properties.

When used herein, unless contradicted by context, the term “Fc region” refers to an antibody region consisting of the two Fc sequences of the heavy chains of an immunoglobulin, wherein said Fc sequences comprise at least a hinge region, a CH2 domain, and a CH3 domain.

The term “Fc region” as used herein, refers to a region comprising, in the direction from the N- to C-terminal end of the antibody, at least a hinge region, a CH2 region and a CH3 region. An Fc region of the antibody may mediate the binding of the immunoglobulin to host tissues or factors, including various cells of the immune system (such as effector cells) and components of the complement system.

The term “hinge region” as used herein refers to the hinge region of an immunoglobulin heavy chain. Thus, for example the hinge region of a human IgG1 antibody corresponds to amino acids 216-230 according to the Eu numbering as set forth in Kabat (Kabat, E. A. et al., Sequences of proteins of immunological interest. 5th Edition—US Department of Health and Human Services, NIH publication No. 91-3242, pp 662,680,689 (1991). However, the hinge region may also be any of the other subtypes as described herein.

The term “CH1 region” or “CH1 domain” as used herein refers to the CH1 region of an immunoglobulin heavy chain. Thus, for example the CH1 region of a human IgG1 antibody corresponds to amino acids 118-215 according to the Eu numbering as set forth in Kabat (ibid). However, the CH1 region may also be any of the other subtypes as described herein.

The term “CH2 region” or “CH2 domain” as used herein refers to the CH2 region of an immunoglobulin heavy chain. Thus, for example the CH2 region of a human IgG1 antibody corresponds to amino acids 231-340 according to the Eu numbering as set forth in Kabat (ibid). However, the CH2 region may also be any of the other subtypes as described herein.

The term “CH3 region” or “CH3 domain” as used herein refers to the CH3 region of an immunoglobulin heavy chain. Thus, for example the CH3 region of a human IgG1 antibody corresponds to amino acids 341-447 according to the Eu numbering as set forth in Kabat (ibid). However, the CH3 region may also be any of the other subtypes as described herein.

The term “full-length” when used in the context of an antibody indicates that the antibody is not a fragment, but contains all of the domains of the particular isotype normally found for that isotype in nature, e.g. the VH, CH1 region, CH2 region, CH3 region, hinge, VL and CL domains for an IgG1 antibody.

As used herein, the terms “binding” or “capable of binding” in the context of the binding of an antibody to a predetermined antigen or epitope typically is a binding with an affinity corresponding to a K_{D} of about 10⁻⁷ M or less, such as about 10⁻⁸ M or less, such as about 10⁻⁹ M or less, about 10⁻¹⁰ M or less, or about 10⁻¹¹ M or even less, when determined using Bio-Layer Interferometry (BLI) or, for instance, when determined using surface plasmon resonance (SPR) technology in a BIAcore 3000 instrument using the antigen as the ligand and the antibody as the analyte. The antibody binds to the predetermined antigen with an affinity corresponding to a K_{D} that is at least ten-fold lower, such as at least 100-fold lower, for instance at least 1,000-fold lower, such as at least 10,000-fold lower, for instance at least 100,000-fold lower than its K_{D} for binding to a non-specific antigen (e.g., BSA, casein) other than the predetermined antigen or a closely-related antigen. The amount with which the affinity is higher is dependent on the K_{D} of the antibody, so that when the K_{D} of the antibody is very low (that is, the antibody is highly specific), then the degree to which the affinity for the antigen is lower than the affinity for a non-specific antigen may be at least 10,000-fold.

The term “k_{d}” (sec⁻¹), as used herein, refers to the dissociation rate constant of a particular antibody-antigen interaction. Said value is also referred to as the k_{off} value.

The term “K_{D}” (M), as used herein, refers to the dissociation equilibrium constant of a particular antibody-antigen interaction.

The term “PD-L1” when used herein, refers to the Programmed Death-Ligand 1 protein. PD-L1 is found in humans and other species, and thus, the term “PD-L1” is not limited to human PD-L1 unless contradicted by context. The human PD-L1 sequences can be found through Genbank accession no. NP_054862.1. The sequence of human PD-L1 is also shown in SEQ ID NO: 25, wherein amino acids 1-18 are predicted to be a signal peptide. The mature polypeptide sequence is provided in SEQ ID NO: 26.

The term “PD-1” when used herein, refers to the human Programmed Death-1 protein, also known as CD279 (UniProtKB Q15116).

The term “programmed cell death-1 (PD-1) pathway” or “PD-1 pathway” refers to the molecular signaling pathway comprising cell surface receptor PD-1 and its ligands PD-L1 and PD-L2. Activation of this pathway induces immune tolerance, while inhibition releases T-cell suppression, which may lead to immune activation.

The term “CD137” as used herein, refers to the human Cluster of Differentiation 137 protein. CD137 (4-1BB), also referred to as TNFRSF9, is the receptor for the ligand TNFSF9/4-1BBL. CD137 is believed to be involved in T cell activation. Human CD137, has UniProt accession number Q07011. The sequence of human CD137 is also shown in SEQ ID NO: 23, wherein amino acids 1-23 are predicted to be a signal peptide. The mature sequence of human CD137 is provided in SEQ ID NO: 24.

The term “treatment” refers to the administration of an effective amount of a therapeutically active antibody optionally in combination with a taxane chemotherapeutic drug as provided by the present invention, with the purpose of easing, ameliorating, arresting or eradicating (curing) symptoms or disease states.

The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The percent identity between two nucleotide or amino acid sequences may e.g. be determined using the algorithm of E. Meyers and W. Miller, Comput. Appl. Biosci 4, 11-17 (1988) which has been incorporated into the ALIGN program (version 2.0), using a PAM120 weight residue table, a gap length penalty of 12 and a gap penalty of 4. In addition, the percent identity between two amino acid sequences may be determined using the Needleman and Wunsch, J. Mol. Biol. 48, 444-453 (1970) algorithm.

In the context of the present invention, the following notations are, unless otherwise indicated, used to describe a mutation: i) substitution of an amino acid in a given position is written as e.g. K409R which means a substitution of a lysine in position 409 of the protein with an arginine; and ii) for specific variants the specific three or one letter codes are used, including the codes Xaa and X to indicate any amino acid residue. Thus, the substitution of lysine with arginine in position 409 is designated as: K409R, and the substitution of lysine with any amino acid residue in position 409 is designated as K409X. In case of deletion of lysine in position 409 it is indicated by K409*.

In the context of the present invention, “inhibition of PD-L1 binding to PD-1” refers to any detectably significant reduction in the binding of PD-L1 to PD-1 in the presence of an antibody capable of binding PD-L1. Typically, inhibition means an at least about 10% reduction, such as an at least about 15%, e.g. an at least about 20%, such as an at least 40% reduction in binding between PD-L1 and PD-1, caused by the presence of an anti-PD-L1 antibody. Inhibition of PD-L1 binding to PD-1 may be determined by any suitable technique. In one embodiment, inhibition is determined as described in Example 6 of WO 2019/025545.

The resistance to, failure to respond to and/or relapse from treatment with a binding agent of the invention and/or other therapeutic agent(s) may be determined according to the Response Evaluation Criteria In Solid Tumors; version 1.1 (RECIST Criteria v1.1). The RECIST Criteria are set forth in the table below.

TABLE 3
Definition of Response (RECIST Criteria v1.1)

| | Category | Criteria |
|---|---|---|
| Based on target | Complete | Disappearance of all target lesions. Any pathological lymph |
| lesions | Response (CR) | nodes must have reduction in short axis to <10 mm. |
| | Partial | ≥30% decrease in the sum of the LD of target lesions, taking |
| | Response (PR) | as reference the baseline sum LD. |
| | Stable | Neither sufficient shrinkage to qualify for PR nor sufficient |
| | Disease (SD) | increase to qualify for PD, taking as reference the smallest |
| | | sum of LDs since the treatment started. |
| | Progressive | ≥20% increase in the sum of the LDs of target lesions, taking |
| | Disease (PD) | as reference the smallest sum of the LDs recorded since the |
| | | treatment started or the appearance of one or more new lesions. |
| Based on non- | CR | Disappearance of all non-target lesions and normalization of |
| target lesions | | tumor marker level. All lymph nodes must be non-pathological |
| | | in size (<10 mm short axis). |
| | SD | Persistence of one or more non-target lesion(s) or/and |
| | | maintenance of tumor marker level above the normal limits. |
| | PD | Appearance of one or more new lesions and/or unequivocal |
| | | progression of existing non-target lesions. |

The “best overall response” is the best response recorded from the start of the treatment until disease progression/recurrence (the smallest measurements recorded since the treatment started will be used as the reference for PD). Subjects with CR or PR are considered to be objective response. Subjects with CR, PR or SD are considered to be in disease control. Subjects with NE are counted as non-responders. The best overall response is the best response recorded from the start of the treatment until disease progression/recurrence (the smallest measurements recorded since the treatment started will be used as the reference for PD). Subjects with CR, PR or SD are considered to be in disease control. Subjects with NE are counted as non-responders.

“Duration of response (DOR)” only applies to subjects whose confirmed best overall response is CR or PR and is defined as the time from the first documentation of objective tumor response (CR or PR) to the date of first PD or death due to underlying cancer.

“Progression-free survival (PFS)” is defined as the number of days from Day 1 in Cycle 1 to the first documented progression or death due to any cause.

“Overall survival (OS)” is defined as the number of days from Day 1 in Cycle 1 to death due to any cause. If a subject is not known to have died, then OS will be censored at the latest date the subject was known to be alive (on or before the cut-off date).

“Taxane chemotherapeutic agent” includes chemotherapeutic agents, which are taxanes as well as chemotherapeutic agents which are derivatives of taxanes, such as semi-synthetic or synthetic taxane derivatives.

In the context of the present invention, the term “treatment regimen” refers to a structured treatment plan designed to improve and maintain health.

In a first aspect the present invention provides a method for reducing or preventing progression of a tumor or treating cancer in a subject, comprising providing to the subject combined treatment with
I) a binding agent comprising a first binding region binding to human CD137, and a second binding region binding to human PD-L1; and
ii) a taxane chemotherapeutic agent.

Preferably, the binding agent and the taxane chemotherapeutic agent are administered to said subject in at least one treatment cycle, such in a plurality pf treatment cycles; e.g. at least 2, at least 3, at least 4, at least 5, at least 6, at least 7, at least 8, at least 9 or at least 10 treatment cycles.

It is further preferred that the binding agent is one wherein the first binding region binds to human CD137 having the sequence set forth in SEQ ID NO: 24, and/or the second binding region binds to human PD-L1 having the sequence set forth in SEQ ID NO: 26.

The binding agent may be one that activates human CD137 when bound thereto and inhibits the binding of human PD-L1 to human PD-1 when bound to PD-L1. I particular embodiments the binding agent used according to the invention binds to human PD-L1, whereby binding of human PD-L1 to human PD-1 is inhibited or blocked, and wherein by binding to human PD-L1 the binding agent also mediates conditional 4-1BB co-stimulation, such as to enhance T-cell and NK cell function.

In the method according to the invention the binding agent may be one, wherein
a) the first binding region comprises, consists of or consist essentially of a heavy chain variable region (VH) comprising the CDR1, CDR2, and CDR3 sequences of SEQ ID NO: 1, and a light chain variable region (VL) comprising the CDR1, CDR2, and CDR3 sequences of SEQ ID NO: 5;
and
b) the second antigen-binding region comprises, consists of or consist essentially of a heavy chain variable region (VH) comprising the CDR1, CDR2, and CDR3 sequences of SEQ ID NO: 8, and a light chain variable region (VL) comprising the CDR1, CDR2, and CDR3 sequences of SEQ ID NO: 12.

In the method according to the invention the binding agent may be one, wherein
a) the first binding region comprises, consists of or consist essentially of a heavy chain variable region (VH) comprising the CDR1, CDR2, and CDR3 sequences set forth in: SEQ ID NO: 2, 3, and 4, respectively, and a light chain variable region (VL) comprising the CDR1, CDR2, and CDR3 sequences set forth in: SEQ ID NO: 6, GAS, 7, respectively;
and
b) the second antigen-binding region comprises, consists of or consist essentially of a heavy chain variable region (VH) comprising the CDR1, CDR2, and CDR3 sequences set forth in: SEQ ID NO: 9, 10, 11 respectively, and a light chain variable region (VL) comprising the CDR1, CDR2, and CDR3 sequences set forth in: SEQ ID NO: 13, DDN, 14, respectively.

Further, in the method according to the invention the binding agent may be one, wherein
a) The first binding region comprises, consists of or consist essentially of a heavy chain variable region (VH) comprising an amino acid sequence having at least 90%, at least 95%, at least 97%, at least 99%, or 100% sequence identity to SEQ ID NO: 1 and a light chain variable region (VL) region comprising an amino acid sequence having at least 90%, at least 95%, at least 97%, at least 99%, or 100% sequence identity to SEQ ID NO: 5;
   and
b) the second binding region comprises, consists of or consist essentially of a heavy chain variable region (VH) comprising an amino acid sequence having at least 90%, at least 95%, at least 97%, at least 99%, or 100% sequence identity to SEQ ID NO: 8 and a light chain variable region (VL) region comprising an amino acid sequence having at least 90%, at least 95%, at least 97%, at least 99%, or 100% sequence identity to SEQ ID NO: 12.

In the method according to the invention the binding agent is one, wherein
a) The first binding region comprises, consists of or consist essentially of a heavy chain variable region (VH) comprising the amino acid sequence set forth in SEQ ID NO: 1 and a light chain variable region (VL) region comprising the amino acid sequence set forth in SEQ ID NO: 5;
and
b) the second binding region comprises, consists of or consist essentially of a heavy chain variable region (VH) comprising the amino acid sequence set forth in SEQ ID NO: 8 and a light chain variable region (VL) region comprising the amino acid sequence set forth in SEQ ID NO: 12.

The binding agent may in particular be an antibody, such as a multispecific antibody, or such as a bispecific antibody.

Also, the binding agent may be in the format of a full-length antibody or an antibody fragment.

It is further preferred that the antibody is a human antibody or a humanized antibody

Each variable region may comprise three complementarity determining regions (CDR1, CDR2, and CDR3) and four framework regions (FR1, FR2, FR3, and FR4).

The complementarity determining regions and the framework regions may be arranged from amino-terminus to carboxy-terminus in the following order: FR1, CDR1, FR2, CDR2, FR3, CDR3, FR4.

The binding agent may comprise
i) a polypeptide comprising, consisting of or consisting essentially of, said first heavy chain variable region (VH) and a first heavy chain constant region (CH), and
ii) a polypeptide comprising, consisting of or consisting essentially of, said second heavy chain variable region (VH) and a second heavy chain constant region (CH).

In the method according to the invention the binding agent may comprise, consist of or consist essentially of
i) a polypeptide comprising said first light chain variable region (VL) and further comprising a first light chain constant region (CL), and
ii) a polypeptide comprising said second light chain variable region (VL) and further comprising a second light chain constant region (CL).

The binding agent may be an antibody comprising a first binding arm and a second binding arm, wherein the first binding arm comprises, consists of or consist essentially of
i) a polypeptide comprising said first heavy chain variable region (VH) and said first heavy chain constant region (CH), and
ii) a polypeptide comprising said first light chain variable region (VL) and said first light chain constant region (CL);
   and the second binding arm comprises, consists of or consist essentially of
iii) a polypeptide comprising said second heavy chain variable region (VH) and said second heavy chain constant region (CH), and
iv) a polypeptide comprising said second light chain variable region (VL) and said second light chain constant region (CL).

The binding agent may comprise, consist of or consist essentially of
i) a first heavy chain and light chain comprising said antigen-binding region capable of binding to CD137, and
ii) a second heavy chain and light chain comprising said antigen-binding region capable of binding PD-L1.

The binding agent may comprise, consist of or consist essentially of
i) a first heavy chain and light chain comprising said antigen-binding region capable of binding to CD137, the first heavy chain comprising a first heavy chain constant region and the first light chain comprising a first light chain constant region; and
ii) a second heavy chain and light chain comprising said antigen-binding region capable of binding PD-L1, the second heavy chain comprising a second heavy chain constant region and the second light chain comprising a second light chain constant region.

Each of the first and second heavy chain constant regions (CH) may comprise one or more of a constant heavy chain 1 (CH1) region, a hinge region, a constant heavy chain 2 (CH2) region and a constant heavy chain 3 (CH3) region, preferably at least a hinge region, a CH2 region and a CH3 region.

Each of the first and second heavy chain constant regions (CHs) may comprise a CH3 region and wherein the two CH3 regions comprise asymmetrical mutations.

In the said first heavy chain constant region (CH) at least one of the amino acids in a position corresponding to a position selected from the group consisting of T366, L368, K370, D399, F405, Y407, and K409 in a human IgG1 heavy chain according to EU numbering may have been substituted, and in said second heavy chain constant region (CH) at least one of the amino acids in a position corresponding to a position selected from the group consisting of T366, L368, K370, D399, F405, Y407, and K409 in a human IgG1 heavy chain according to EU numbering may have been substituted. In particular embodiments, the first and the second heavy chains are not substituted in the same positions.

The binding agent may be one, wherein (i) the amino acid in the position corresponding to F405 in a human IgG1 heavy chain according to EU numbering is L in said first heavy chain constant region (CH), and the amino acid in the position corresponding to K409 in a human IgG1 heavy chain according to EU numbering is R in said second heavy chain constant region (CH), or (ii) the amino acid in the position corresponding to K409 in a human IgG1 heavy chain according to EU numbering is R in said first heavy chain, and the amino acid in the position corresponding to F405 in a human IgG1 heavy chain according to EU numbering is L in said second heavy chain.

In the method according to the invention, the binding agent may be one which induces Fc-mediated effector function to a lesser extent compared to another antibody comprising the same first and second antigen binding regions and two heavy chain constant regions (CHs) comprising human IgG1 hinge, CH2 and CH3 regions.

In particular, the method may use a binding agent, wherein said first and second heavy chain constant regions (CHs) are modified so that the antibody induces Fc-mediated effector function to a lesser extent compared to an antibody which is identical except for comprising non-modified first and second heavy chain constant regions (CHs). In particular, each non-modified first and second heavy chain constant regions (CHs) or both non-modified first and second CHs may comprise, consists of or consist essentially of the amino acid sequence set forth in SEQ ID NO: 15.

The Fc-mediated effector function may be determined by measuring binding of the binding agent to Fcγ receptors, binding to C1q, or induction of Fc-mediated cross-linking of Fcγ receptors. In particular, the Fc-mediated effector function may be determined by measuring binding of the binding agent to C1q.

The first and second heavy chain constant regions of the binding agent may have been modified so that binding of C1q to said antibody is reduced compared to a wild-type antibody, preferably reduced by at least 70%, at least 80%, at least 90%, at least 95%, at least 97%, or 100%, wherein C1q binding is preferably determined by ELISA.

The binding agent used in the method provided herein may be one wherein, in at least one of said first and second heavy chain constant regions (CH), one or more amino acids in the positions corresponding to positions L234, L235, D265, N297, and P331 in a human IgG1 heavy chain according to EU numbering, are not L, L, D, N, and P, respectively.

In the binding agent used according to the invention the positions corresponding to positions L234 and L235 in a human IgG1 heavy chain according to EU numbering may be F and E, respectively, in said first and second heavy chains.

In particular, the positions corresponding to positions L234, L235, and D265 in a human IgG1 heavy chain according to EU numbering may be F, E, and A, respectively, in said first and second heavy chain constant regions (HCs).

The binding agent used in the method according to the invention may be one, wherein the positions corresponding to positions L234 and L235 in a human IgG1 heavy chain according to EU numbering of both the first and second heavy chain constant regions are F and E, respectively, and wherein (i) the position corresponding to F405 in a human IgG1 heavy chain according to EU numbering of the first heavy chain constant region is L, and the position corresponding to K409 in a human IgG1 heavy chain according to EU numbering of the second heavy chain is R, or (ii) the position corresponding to K409 in a human IgG1 heavy chain according to EU numbering of the first heavy chain constant region is R, and the position corresponding to F405 in a human IgG1 heavy chain according to EU numbering of the second heavy chain is L.

The binding agent used in the method according to the invention may be one, wherein the positions corresponding to positions L234, L235, and D265 in a human IgG1 heavy chain according to EU numbering of both the first and second heavy chain constant regions are F, E, and A, respectively, and wherein (i) the position corresponding to F405 in a human IgG1 heavy chain according to EU numbering of the first heavy chain constant region is L, and the position corresponding to K409 in a human IgG1 heavy chain according to EU numbering of the second heavy chain constant region is R, or (ii) the position corresponding to K409 in a human IgG1 heavy chain according to EU numbering of the first heavy chain is R, and the position corresponding to F405 in a human IgG1 heavy chain according to EU numbering of the second heavy chain is L.

The binding agent used in the method according to the invention may be one, wherein the constant region of said first and/or second heavy chain comprises or consists essentially of or consists of an amino acid sequence selected from the group consisting of
a) the sequence set forth in SEQ ID NO: 15 [IgG1-FC],
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 10 substitutions, such as at the most 9 substitutions, at the most 8, at the most 7, at the most 6, at the most 5, at the most 4, at the most 3, at the most 2 or at the most 1 substitution compared to the amino acid sequence defined in a) or b).

The binding agent used in the method according to the invention may be one, wherein the constant region of said first or second heavy chain, such as the second heavy chain, comprises or consists essentially of or consists of an amino acid sequence selected from the group consisting of
a) the sequence set forth in SEQ ID NO: 16 [IgG1-F405L],
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 9 substitutions, such as at the most 8, at the most 7, at the most 6, at the most 5, at the most 4, at the most 3, at the most 2 or at the most 1 substitution compared to the amino acid sequence defined in a) or b).

The binding agent used in the method according to the invention may be one, wherein the constant region of said first or second heavy chain, such as the first heavy chain comprises or consists essentially of or consists of an amino acid sequence selected from the group consisting of
a) the sequence set forth in SEQ ID NO: 17 [IgG1-F409R]
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 10 substitutions, such as at the most 9 substitutions, at the most 8, at the most 7, at the most 6, at the most 5, at the most 4 substitutions, at the most 3, at the most 2 or at the most 1 substitution compared to the amino acid sequence defined in a) or b).

The binding agent used in the method according to the invention may be one, wherein the constant region of said first and/or second heavy chain comprises or consists essentially of or consists of an amino acid sequence selected from the group consisting of
a) the sequence set forth in SEQ ID NO: 18 [IgG1-Fc_FEA],
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 7 substitutions, such as at the most 6 substitutions, at the most 5, at the most 4, at the most 3, at the most 2 or at the most 1 substitution compared to the amino acid sequence defined in a) or b).

The binding agent used in the method according to the invention may be one, wherein the constant region of said first and/or second heavy chain, such as the second heavy chain, comprises or consists essentially of or consists of an amino acid sequence selected from the group consisting of
a) the sequence set forth in SEQ ID NO: 20 [IgG1-Fc_FEAL],
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 6 substitutions, such as at the most 5 substitutions, at the most 4 substitutions, at the most 3, at the most 2 or at the most 1 substitution compared to the amino acid sequence defined in a) or b).

The binding agent used in the method according to the invention may be one, wherein the constant region of said first and/or second heavy chain, such as the first heavy chain, comprises or consists essentially of or consists of an amino acid sequence selected from the group consisting of
a) the sequence set forth in SEQ ID NO: 19 [IgG1-Fc_FEAR]
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 6 substitutions, such as at the most 5 substitutions, at the most 4, at the most 3, at the most 2 or at the most 1 substitution compared to the amino acid sequence defined in a) or b).

The binding agent used in the method according to the invention may comprise a kappa (κ) light chain constant region.

The binding agent used in the method according to the invention may comprises comprise a lambda (λ) light chain constant region.

The binding agent used in the method according to the invention may be one, wherein said first light chain constant region is a kappa (κ) light chain constant region.

The binding agent used in the method according to the invention may be one, wherein said second light chain constant region is a lambda (λ) light chain constant region.

The binding agent used in the method according to the invention may be one, wherein said first light chain constant region is a lambda (λ) light chain constant region.

The binding agent used in the method according to the invention may be one, wherein second light chain constant region is a kappa (κ) light chain constant region.

The binding agent used in the method according to the invention may be one, wherein the kappa (κ) light chain comprises an amino acid sequence selected from the group consisting of
a) the sequence set forth in SEQ ID NO: 21,
b) a subsequence of the sequence in a), such as a subsequence wherein 1, 2, 3, 4, 5, 6, 7, 8, 9 or 10 consecutive amino acids has/have been deleted, starting from the N-terminus or C-terminus of the sequence defined in a); and
c) a sequence having at the most 10 substitutions, such as at the most 9 substitutions, at the most 8, at the most 7, at the most 6, at the most 5, at the most 4 substitutions, at the most 3, at the most 2 or at the most 1 substitution, compared to the amino acid sequence defined in a) or b).

The binding agent used in the method according to the invention may be one, wherein the lambda (λ) light chain comprises an amino acid sequence selected from the group consisting of
a) the sequence set forth in SEQ ID NO: 22,
b) a subsequence of the sequence in a), such as a subsequence wherein 1, 2, 3, 4, 5, 6, 7, 8, 9 or 10 consecutive amino acids has/have been deleted, starting from the N-terminus or C-terminus of the sequence defined in a); and
c) a sequence having at the most 10 substitutions, such as at the most 9 substitutions, at the most 8, at the most 7, at the most 6, at the most 5, at the most 4 substitutions, at the most 3, at the most 2 or at the most 1 substitution, compared to the amino acid sequence defined in a) or b).

The binding agent may be of an isotype selected from the group consisting of IgG1, IgG2, IgG3, and IgG4.

In particular, the binding agent may be a full-length IgG1 antibody.

In currently preferred embodiments, the antibody is of the IgG1m(f) allotype.

It is further preferred that said binding agent or antibody is acasunlimab or a biosimilar thereof.

Taxanes are a class of diterpenes several of which are in use or is being developed for use as chemotherapeutic agents including paclitaxel and docetaxel that produce antitumor activity by causing stabilization of cellular microtubules, thereby inhibiting cell division. Taxane has the molecular formula C₂₀H₃₆ and the following chemical structure:

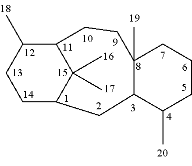 [SMILES: CC1CCCC2(C)CCC3C(C)CCC(CC12)C3(C)C]

The taxane chemotherapeutic agent may in particular be selected from the group consisting of: Docetaxel, Paclitaxel, Cabazitaxel and Tesetaxel.

Paclitaxel was first approved by the FDA in 1992 and is marketed under the brand/trade names Taxol, Abraxane. The molecular formula of paclitaxel is: C₄₇H₅₁NO₁₄ and it has the following chemical structure:

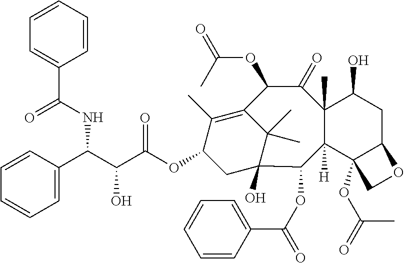 [SMILES: CC(=O)O[C@H]1C(=O)[C@@]2(C)[C@H]([C@H](OC(=O)c3ccccc3)[C@]3(O)C[C@H](OC(=O)[C@H](O)[C@@H](NC(=O)c4ccccc4)c4ccccc4)C(C)=C1C3(C)C)[C@]1(OC(C)=O)CO[C@@H]1C[C@@H]2O]

Docetaxel was first approved by the FDA in 1996 and is marketed under the brand/trade name Taxotere. The molecular formula of docetaxel is C₄₃H₅₃NO₁₄ and it has the following chemical structure

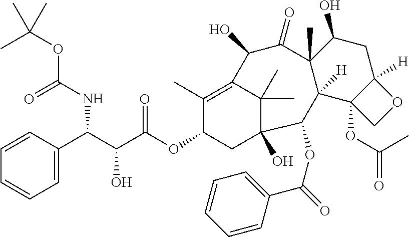

Cabazitaxel was first approved by the FDA in 2010 and is marketed under the brand/trade name Jevtana. The molecular formula of cabaztaxel is C₄₅H₅₇NO₁₄ and it has the following chemical structure:

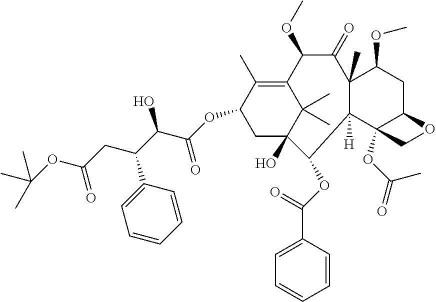 [SMILES: CO[C@H]1C(=O)[C@]2(C)[C@@H](OC)C[C@H]3OC[C@@]3(OC(C)=O)[C@H]2[C@H](OC(=O)c2ccccc2)[C@]2(O)C[C@H](OC(=O)[C@H](O)[C@@H](CC(=O)OC(C)(C)C)c3ccccc3)C(C)=C1C2(C)C]

Tesetaxel is a semi-synthetic, orally bioavailable taxane derivative with the chemical formula C₄₆H₅₀FN₃O₁₃. The chemical formula of tesetaxel is:

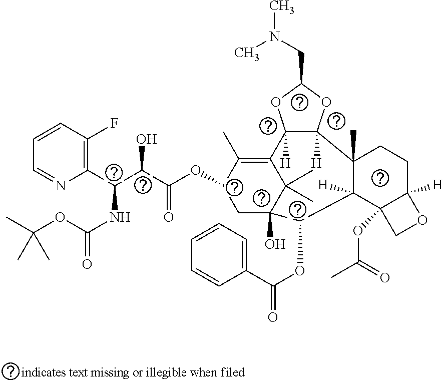 [SMILES: CC(=O)O[C@@]12CO[C@@H]1CC[C@@]1(C)[C@@H]3O[C@H](CN(C)C)O[C@@H]3C3=C(C)[C@@H](OC(=O)[C@H](O)[C@@H](NC(=O)OC(C)(C)C)c4ncccc4F)C[C@@](O)([C@@H](OC(=O)c4ccccc4)[C@@H]12)C3(C)C]

In currently preferred embodiments the taxane chemotherapeutic drug is docetaxel.

The amount of binding agent administered in each dose and/or in each treatment cycle may be
a) about 0.3-15 mg/kg body weight or about 25-1200 mg in total; and/or
b) about 2.1×10⁻⁹−1.2×10⁻⁷ mol/kg body weight or about 1.7×10⁻⁷−8.1×10⁻⁶ mol in total.

The amount of binding agent administered in each dose and/or in each treatment cycle may also be
a) about 0.3-10 mg/kg body weight or about 25-800 mg in total; and/or
b) about 2.1×10⁻⁹−6.8×10⁻⁸ mol/kg body weight or about 1.7×10⁻⁷−5.4×10⁻⁶ mol in total.

Further, the amount of binding agent administered in each dose and/or in each treatment cycle may be
a) about 0.3-5 mg/kg body weight or about 25-400 mg in total; and/or
b) about 2.1×10⁻⁹−3.4×10⁻⁸ mol/kg body weight or about 1.7×10⁻⁷−2.7×10⁻⁶ mol in total.

In particular embodiments the amount of binding agent administered in each dose and/or in each treatment cycle may be
a) about 1.25 mg/kg body weight or about 100 mg in total; and/or
b) about 8.5×10⁻⁹ mol/kg body weight or about 6.8×10⁻⁷ mol in total.

The amount of taxane chemotherapeutic agent administered in each dose and/or in each treatment cycle may be about 10-200 mg/m², such as 20-40 mg/m², 30-50 mg/m², 40-100 mg/m², 50-100 mg/m², 50-80 mg/m², 50-70 mg/m², 50-60 mg/m², 50-110 mg/m², 60-100 mg/m², 60-100 mg/m², 60-90 mg/m², 70-80 mg/m², 80-200 mg/m², 90-180 mg/m², 90-110 mg/m², 100-175 mg/m², or such as a bout 170-180 mg/m².

In particular embodiments, the taxane chemotherapeutic agent is docetaxel and the amount administered in each dose and/or in each treatment cycle is about 50-60 mg/m², such as about 55 mg/m².

In other embodiments, the taxane chemotherapeutic agent is docetaxel and the amount administered in each dose and/or in each treatment cycle is about 70-80 mg/m², such as about 75 mg/m².

It is to be understood that in treatment regimens according to the invention, the amount of taxane chemotherapeutic agent may initially be dosed as set forth above and may then be subject to dose reduction in order to avoid or reduce adverse effects. The dose reduction may in particular be according to label and/or according to established local clinical practice. For instance, while the amount of taxane chemotherapeutic agent administered in each dose and/or in each treatment cycle may initially be about 50-60 mg/m², such as about 55 mg/m² or about 70-80 mg/m², such as about 75 mg/m², the amount administered in each dose and/or in each treatment cycle may later be reduced to 20-40 mg/m², such as to 35 mg/m².

The binding agent and/or the taxane chemotherapeutic agent used according to the present invention may in particular be administered by systemic administration.

Preferably, the binding agent and/or the taxane chemotherapeutic agent is/are administered to said subject by intravenous injection or infusion.

At least one dose of said binding agent and at least one dose of said taxane chemotherapeutic drug may be administered in each treatment cycle.

Each treatment cycle treatment cycle may be one week (7 days), two weeks (14 days), three weeks (21 days) or four weeks (28 days).

In particular embodiments of the invention, each dose is administered or infused once every week, once every second week (1Q2W), once every third week (1Q3W) or once every fourth week (1Q4W).

The binding agent and the taxane chemotherapeutic agent may be administered on the same day.

In particular embodiments, each dose of said binding agent is infused over a minimum of 30 minutes, such as over a minimum of 60 minutes, a minimum of 90 minutes, a minimum of 120 minutes or a minimum of 240 minutes.

In the method according to the invention administration of the binding agent preferably precedes administration of the taxane chemotherapeutic agent by at least 30 minutes, such as by at least 1 hour or such as at least 2 hours.

In relation to the dosing of binding agent and taxane chemotherapeutic agent according to the invention it is to be understood that administration of multiple small doses over a short time span; e.g. within 2-24 hours, such as 2-12 hours or on the same day, may be considered to be equal to administration of a larger single dose. For example, infusion of 25 mg binding agent four times on the same day may be considered equivalent to a single dose of 100 mg provided by uninterrupted infusion.

In a currently preferred treatment regimen one dose of said binding agent and/or one dose of said taxane chemotherapeutic agent is/are administered on day 1 of each treatment cycle.

In particular, one dose of said binding agent may be administered every third week (1Q3W), such as on day 1 on each three-week treatment cycle.

It is currently preferred that one dose of said taxane chemotherapeutic agent is administered every third week (1Q3W), such as on day 1 on each three-week treatment cycle.

The method according to any one of the preceding claims, wherein each dose of taxane chemotherapeutic agent is preceded by premedication, such as steroid premedication, e.g. to reduce the incidence and severity of fluid retention, as well as the severity of hypersensitivity reactions. The steroid premedication may for instance be with an oral corticosteroid; e.g. administration of about 8 mg dexamethasone 2 times a day for 3 days starting 1 day prior to administration of the taxane chemotherapeutic agent.

The subject to be treated according to the present invention is preferably a human subject.

The tumor or cancer is preferably a solid tumor.

The tumor or cancer may be selected from the group consisting of melanoma, ovarian cancer, lung cancer (e.g. non-small cell lung cancer (NSCLC), colorectal cancer, head and neck cancer, gastric cancer, breast cancer, renal cancer, urothelial cancer, bladder cancer, esophageal cancer, pancreatic cancer, hepatic cancer, thymoma and thymic carcinoma, brain cancer, glioma, adrenocortical carcinoma, thyroid cancer, other skin cancers, sarcoma, multiple myeloma, leukemia, lymphoma, myelodysplastic syndromes, ovarian cancer, endometrial or cancer, prostate cancer, penile cancer, cervical cancer, Hodgkin's lymphoma, non-Hodgkin's lymphoma, Merkel cell carcinoma and mesothelioma.

In particular embodiments, the tumor or cancer is selected from the group consisting of lung cancer (e.g. non-small cell lung cancer (NSCLC), urothelial cancer (cancer of the bladder, ureter, urethra, or renal pelvis), endometrial cancer (EC), breast cancer (e.g. triple negative breast cancer (TNBC)), squamous cell carcinoma of the head and neck (SCCHN) (e.g. cancer of the oral cavity, pharynx or larynx) and cervical cancer.

The tumor or cancer may in particular be a lung cancer.

The lung cancer may be a non-small cell lung cancer (NSCLC), such as a squamous or a non-squamous NSCLC.

Lung cancer is the most common malignancy and the most common cause of cancer death worldwide. Non-small cell lung cancer (NSCLC) accounts for 85-90% of all lung cancer cases (Jemal et al., 2011). The five-year survival rate for NSCLC is approximately 18% (SEER, 2018). Major histological subtypes of NSCLC include adenocarcinoma, squamous cell carcinoma, adenosquamous carcinoma, large cell carcinoma, carcinoid tumors, and other less common subtypes, with adenocarcinoma being the most common.

Standard of care for patients with advanced or metastatic NSCLC who have progressed on targeted therapy or are no longer candidates for targeted therapy typically includes platinum-based chemotherapy. Platinum combinations have generated an overall response rate (ORR) of approximately 25-35%, a time to progression (TTP) of 4 6 months, and median survival of 8-10 months.

Tumor gene mutations/alterations have been identified and have impact on therapy selection. Identification of specific mutations or alterations in genes within the tumor, such as anaplastic lymphoma kinase (ALK), epidermal growth factor receptor (EGFR), c-ROS oncogene 1 (ROS1), BRAF, KRAS, and program death ligand-1 (PD-L1), aids the selection of potentially efficacious targeted therapies, while avoiding the use of therapies unlikely to provide clinical benefit (NCCN, 2018c). Activating sensitizing EGFR mutations are predictive for response to the EGFR Tyrosine Kinase Inhibitors (TKIs) (e.g., gefitinib, erlotinib, afatinib, and osimertinib). Similarly, TKIs (e.g., alectinib, ceritinib, and crizotinib) are effective therapies for ALK and ROS1 mutations and are also approved as first-line therapy for the respective mutations. Checkpoint inhibitor antibodies (e.g., pembrolizumab and nivolumab) that block the PD 1 and PD-L1 interaction have also been shown as effective treatment alone or in combination with chemotherapy for the treatment of patients with advanced or metastatic NSCLC whose tumors express PD-L1.

Despite multiple treatment options, patients with stage IV NSCLC ultimately have a poor prognosis and lung cancer remains the leading cause of cancer death for both men and women. The treatment rate diminishes with each line of therapy, as patients succumb to their cancer or experience deterioration of their health that makes further treatment impossible.

The lung cancer may be NSCLC, which does not have an epidermal growth factor (EGFR)-sensitizing mutation and/or anaplastic lymphoma (ALK) translocation/ROS1 rearrangement. EGFR sensitizing mutations refers to mutations that confer sensitivity to EGFR tyrosine kinase inhibitors (TKIs), such as approved tyrosine kinase inhibitors erlotinib, osimertinib, gefintinib, olmutinib, nazartinib and avitinib.

The epidermal growth factor receptor (EGFR) amino acid sequence is provided herein as SEQ ID NO: 27.

The sensitizing mutation in the epidermal growth factor receptor (EGFR) amno acid sequence may be selected from the group consisting of:
i) An in-frame deletion and optionally insertion of one or more amino acids at position 746-751, such as any of the deletions and insertions defined in table 4,
ii) Substitution of a single amino acid at any one of positions 709, 715, 719, 720, 768, 858 and 861 such as any of the deletions and insertions defined in table 5, and
iii) An in-frame duplication and/or insertion selected from the duplications/insertions defined in Table 6;
amino acid numbering referring to the numbering of amino acids in SEQ ID NO: 27.

TABLE 4
In-frame deletions within exon 19 of the human EGFR
gene (Adapted from Shigematsu et al., Clinical and
Biological Features Associated With Epidermal Growth
Factor Receptor Gene Mutations in Lung Cancers, JNCI:
Journal of the National Cancer Institute, Volume 97,
Issue 5, 2 Mar. 2005). del = deletion; ins = insertion.

| Designation | Amino acid change |
|---|---|
| Δ1 | E746-A750 del |
| Δ2 | E746-A750 del |
| Δ3 | L747-T751 del |
| Δ4 | L747-E749 del P ins |
| Δ5 | L747-T750 del P ins |
| Δ6 | L747-S752 del S ins |
| Δ7 | E746-T751 del V ins |
| Δ8 | L747-S752 del |
| Δ9 | E746-T751 del I ins |
| Δ10 | E746-A750 del V ins |
| Δ11 | L747-S752 del Q ins |

TABLE 5
Single nucleotide substitutions and resulting amino acid
changes within exon 21 of the human EGFR gene (Adapted
from Shigematsu et al., Clinical and Biological Features
Associated With Epidermal Growth Factor Receptor Gene
Mutations in Lung Cancers, JNCI: Journal of the National
Cancer Institute, Volume 97, Issue 5, 2 Mar. 2005).

| Designation | Amino acid change |
|---|---|
| M1 | L858R |
| M2 | E709V |
| M3 | I715S |
| M4 | G719C |
| M5 | G719S |
| M6 | G719A |
| M7 | S720F |
| M8 | S768I |
| M9 | L861Q |

TABLE 6
In-frame duplications and/or insertions within exon 20 of the human
EGFR gene (Adapted from Shigematsu et al., Clinical and Biological
Features Associated With Epidermal Growth Factor Receptor Gene
Mutations in Lung Cancers, JNCI: Journal of the National Cancer
Institute, Volume 97, Issue 5, 2 Mar. 2005). ins = insertion.

| Designation | Amino acid change |
|---|---|
| D1 | ASV770-772 ins |
| D2 | H774 ins |
| D3 | G771 ins |
| D4 | CV770-771 ins |
| D5 | NP773-774 ins, H775Y |
| D6 | PH774-775 ins |
| D7 | NPH774-776 ins |
| D8 | HV775-776 ins |

The non-small cell lung cancer may be characterized by, and/or the subject receiving the treatment may have, at least one mutation in the EGFR amino acid sequence selected from L747S, D761Y, T790M, C797S, T854A, such as T790M, C797S, D761Y, and double mutations T790M/D761Y and T790/C797S; amino acid numbering referring to the numbering of amino acids in SEQ ID NO: 27.

The non-small cell lung cancer may be characterized by expression of an epidermal growth factor receptor (EGFR) selected form the group consisting of:
i. a wild-type human EGFR; e.g. a human EFGR that comprises the sequence set forth in SEQ ID NO: 27 or a mature polypeptide thereof; and
ii. a human EGFR which is a variant of the EGFR in item i and which, when compared with the EGFR in item I, does not have any sensitizing mutations.

The non-small cell lung cancer may be a cancer which is not characterized by a sensitizing epidermal growth factor receptor (EGFR) mutation selected from the group consisting of:
i) An in-frame deletion and optionally insertion of one or more amino acids at position 746-751, such as any of the deletions and insertions defined in table 4,
ii) Substitution of a single amino acid at any one of positions 709, 715, 719, 720, 768, 858 and 861 such as any of the deletions and insertions defined in table 5, and
iii) An In-frame duplication and/or insertion selected from the duplications/insertions defined in Table 6;
   amino acid numbering referring to the numbering of amino acids in SEQ ID NO: 27. Likewise, the subject receiving treatment according to the invention may be a subject that does not have such a sensitizing EGFR mutation.

The non-small cell lung cancer may be a cancer, which is not characterized by a mutation in the EGFR amino acid sequence selected from L747S, D761Y, T790M, C797S, T854A, such as from T790M, C797S, D761Y, and double mutations T790M/D761Y and T790/C797S; amino acid numbering referring to the numbering of amino acids in SEQ ID NO: 27. Likewise, the subject receiving treatment according to the invention may be a subject that does not have any of the said mutations.

The non-small cell lung cancer and/or the subject receiving treatment according to the invention may be characterized by having a mutation in the gene coding for the ALK tyrosine kinase (ALK), which leads to rearrangement of the gene coding for ALK (UniProt Q9UM73) with a gene coding for a fusion partner, to form a fusion oncogene.

The non-small cell lung cancer may be characterized by, and/or the subject receiving treatment according to the invention may have a mutation in the gene coding the ALK, said mutation leading to rearrangement of the gene coding for ALK with the gene (EML4) coding for Echinoderm microtubule-associated protein-like 4 (EMAPL4) (UniProt Q9HC35) (and formation of an EML4-ALK fusion oncogene).

The non-small cell lung cancer may be characterized by, and/or the subject receiving treatment according to the invention may have a mutation in the gene coding for the ALK tyrosine kinase (ALK), leading to rearrangement of the gene coding for the ALK with a gene selected from the group consisting of
i. KIF5B coding for Kinesin-1 heavy chain (KINH) (UniProt P33176),
ii. KLC1 coding for Kinesin light chain 1 (KLC1) (UniProt Q07866),
iii. TFG coding for Protein TFG (UniProt Q92734),
iv. TPR coding for Nucleoprotein TPR (UniProt P12270),
v. HIP1 coding for Huntington-interacting protein 1 (HIP-1) (UniProtKB—O00291),
vi. STRN coding for Striatin (UniProtKB—O43815),
vii. DCTN1 coding for dynactin subunit 1 (UniProt Q14203),
viii. SQSTM1 coding for sequestosome-1 (UniProtKB—Q13501),
ix. NPM1 coding for nucleophosmin (UniProt P06748),
x. BCL11A coding for B-cell lymphoma/leukemia 11A (UniProt Q9H165), and
xi. BIRC6 coding for baculoviral IAP repeat-containing protein (UniProt Q13490);
   and formation of the respective fusion oncogene selected from the group consisting of a KIF5B-ALK fusion oncogene, a KLC1-ALK fusion oncogene, a TFG-ALK fusion oncogene, a TPR-ALK fusion oncogene, an HIP1-ALK fusion oncogene, a STRN-ALK fusion oncogene, a DCTN1-ALK fusion oncogene, a SQSTM1-ALK fusion oncogene, a NPM1-ALK fusion oncogene, a BCL11A-ALK fusion oncogene and a BIRC6-ALK fusion oncogene.

The non-small cell lung cancer may be characterized by expression of a wild-type human ALK tyrosine kinase; e.g. a human ALK tyrosine kinase that comprises the sequence provided under UniProt Q9HC35 or a mature polypeptide thereof.

The non-small cell lung cancer may be characterized by not having a mutation in the gene coding for the ALK tyrosine kinase (ALK), leading to rearrangement of ALK with fusion partner to form a fusion oncogene and/or the subject does not have such a mutation.

The non-small cell lung cancer may be characterized by not having a mutation in the gene coding for the ALK tyrosine kinase (ALK), leading to rearrangement of the gene (EML4) coding for Echinoderm microtubule-associated protein-like 4 (EMAPL4) (UniProt Q9HC35) with ALK (UniProt Q9HC35) and formation of an EML4-ALK fusion oncogene and/or the subject may be a subject that does not have such a mutation.

The non-small cell lung cancer may be characterized by not having a mutation in any gene selected from the group consisting of the gene coding for the ALK tyrosine kinase (ALK), the gene (EML4) coding for Echinoderm microtubule-associated protein-like 4 (EMAPL4) (UniProt Q9HC35).

The non-small cell lung cancer may be a cancer that is not characterized by a mutation selected from the group consisting of
a sensitizing epidermal growth factor receptor (EGFR) mutation,
a mutation in the gene coding for the ALK tyrosine kinase (ALK), leading to rearrangement of EML4 with ALK and formation of an EML4-ALK fusion oncogene,
a mutation in the EGFR amino acid sequence, which induces or confers resistance of said subject to one or more EGFR tysrosine kinase inhibitors (EGFR-TKIs); and
   the subject may have been treated with a programmed cell death-1 (PD-1)/programmed cell death-1 (PD-1) inhibitor (e.g. nivolumab, genolimzumab, atezolizumab, durvalumab or avelumab) or with chemotherapy (e.g. chemotherapy comprising platinum, a taxane, pemetrexed and/or gemcitabine) and may have failed with such previous treatment.

The non-small cell lung cancer may be characterized by a mutation selected from the group consisting of
a sensitizing epidermal growth factor receptor (EGFR) mutation,
a mutation in the EGFR amino acid sequence, which induces or confers resistance of said subject to one or more EGFR tysrosine kinase inhibitors (EGFR-TKIs),
a mutation in the gene coding for the ALK tyrosine kinase (ALK), leading to rearrangement of EML4 with ALK and formation of an EML4-ALK fusion oncogene; and
   the subject may have been treated with an EGFR inhibitor (e.g. erlotinib, osimertinib, gefintinib, olmutinib, nazartinib and avitinib) or with a PD-1/PD-L1 inhibitor (e.g. nivolumab, genolimzumab, atezolizumab, durvalumab or avelumab) and has failed with such previous treatment.

The subject has received up to four prior systemic treatment regimens for advanced/metastatic disease to treat the lung cancer and has experienced disease progression on or after last prior systemic treatment, such as disease progression determined by radiography.

Before receiving treatment according to the present invention, the subject has received platinum-based chemotherapy to treat the lung cancer. Alternatively, the subject may not be eligible for platinum-based therapy and have received alternative chemotherapy, e.g., a treatment with gemcitabine-containing regimen.

The subject may have received prior treatment with checkpoint inhibitor(s) to treat the lung cancer, such as agent(s) targeting programmed cell death-1 (PD-1)/programmed death-ligand 1 (PD-L1), such as a PD-1/PD-L1 inhibitor. Preferably, subjects must have received only one prior treatment with PD-1/PD-L1 inhibitor alone or in combination.

In particular, the subject may have experienced disease progression on or after treatment with checkpoint inhibitor(s), such as agent(s) targeting PD-1/PD-L, such as a PD-1/PD-L1 inhibitor. Further, the subject has experienced disease progression on or after last prior treatment with checkpoint inhibitor(s), such as agent(s) targeting PD-1/PD-L, such as a PD-1/PD-L1 inhibitor.

The inhibitor of PD-1 and/or PD-L1 may in particular comprise an antibody, or antigen-binding fragment thereof, capable of binding to PD-L1.

Known inhibitors of PD-1 and/or PD-L1 include pembrolizumab (Merck & Co), CBT-501 (genolimzumab; Genor Bio/CBT Pharma), nivolumab (BMS), REGN2810 (Cemiplimab; Regeneron), BGB-A317 (Tislelizumab; BeiGene/Celgene), Amp-514 (MEDI0680) (Amplimmune), TSR-042 (Dostarlimab; Tesaro/AnaptysBio), JNJ-63723283/JNJ-3283 (Johnson & Johnson), PF-06801591 (Pfizer), JS-001 (Tripolibamab/Toripalimab; Shanghai Junshi Bio), SHR-1210/INCSHR-1210 (Camrelizumab; Incyte corp), PDR001 (Spartalizumab; Novartis), BCD-100 (BioCad), AGEN2034 (Agenus), IBI-308 (Sintilimab; Innovent Biologics), RG7446/MPDL-3280A (atezolizumab; Roche), MSB-0010718C (avelumab; Merck Serono/Pfizer) and MEDI-4736 (durvalumab; AstraZeneca), KN-035 (envafolimab; 3DMed/Alphamab Co.).

In particular, the subject may have experienced disease progression on or after last prior systemic treatment, such as disease progression determined by radiography.

Alternatively, the subject receiving treatment according to the invention may be one that has not received prior treatment with checkpoint inhibitor(s) to treat said lung cancer, such as agent(s) targeting PD-1/PD-L, such as a PD-1/PD-L1 inhibitor; e.g. any of the PD-1/PD-L1 inhibitors recited above.

In other embodiments the tumor or cancer is an endometrial cancer. In the US as well as other developed countries, uterine endometrial cancer (EC) was the most common gynecological malignancy, with increasing incidence globally. In the United States, there were estimated 60,000 new cases reported and over 10,000 deaths in 2016. Worldwide in 2012, 527,600 women were diagnosed with uterine EC. A majority of EC cases are identified at an early stage and are treated with surgery with or without radiotherapy or chemotherapy. However, patients with advanced disease have a poorer prognosis with a 5-year survival rate of less than 50% for patients with lymph node metastases and less than 20% for patients with peritoneal or distant metastases.

Multiagent chemotherapy is the preferred treatment for metastatic, recurrent, or high-risk disease; however, there is no consensus on a standard regimen. Carboplatin and paclitaxel are increasingly used in the first-line setting for advanced/metastatic or recurrent EC. Response rates with carboplatin and paclitaxel range from 40% to 62% with an OS of approximately 13 to 29 months. Patients who progress on combination therapy or who are unable to tolerate multi-agent chemotherapy may receive single-agent therapy, however, chemotherapeutic options in this setting have produced only modest activity, especially in the second-line setting and beyond. Single agent response rates range from 21% to 36% in the first-line setting and 4% to 27% in the second-line setting (NCCN, 2018d).

Most recently, pembrolizumab has demonstrated anti-tumor activity in patients with locally advanced or metastatic PD-L1 positive EC who experienced progression on or after standard therapy

In particular, the subject or the endometrial cancer treated according to the invention may have epithelial endometrial histology including: endometrioid, serous, squamous, clear-cell carcinoma, or carcinosarcoma.

The subject may have received up to four prior systemic treatment regimens for advanced/metastatic disease to treat said endometrial cancer and may have experienced disease progression on or after last prior systemic treatment, such as disease progression determined by radiography.

The subject may be one that has not received prior treatment with checkpoint inhibitor(s) to treat said endometrial cancer, such as agent(s) targeting PD-1/PD-L, such as a PD-1/PD-L1 inhibitor; e.g. a PD-1/PD-L1 inhibitor selected from the list of PD-1/PD-L1 inhibitors above.

According to other embodiments, the tumor or cancer is an urothelial cancer, including cancer of the bladder, ureter, urethra, or renal pelvis.

The subject may have received up to four prior systemic treatment regimens for advanced/metastatic disease to treat said urothelial cancer and may have experienced disease progression on or after last prior systemic treatment, such as disease progression determined by radiography.

The subject may have received prior treatment with checkpoint inhibitor(s) to treat said urothelial cancer, such as agent(s) targeting PD-1/PD-L, such as a PD-1/PD-L1 inhibitor; e.g. any one of the PD-1/PD-L1 inhibitors listed above.

Further, the subject may be one that has received platinum-based chemotherapy to treat said urothelial cancer; i.e. chemotherapy with an agent which is a are coordination complex of platinum. Examples of platinum-based chemotherapy include treatment with cisplatin, oxaliplatin, and carboplatin.

The subject may be one that is not eligible for platinum-based therapy and has received alternative chemotherapy, e.g., a treatment with gemcitabine-containing regimen.

In other embodiments according to the invention, the tumor or cancer is a breast cancer, such as a triple negative breast cancer (TNBC). TNBC generally refers to breast cancers that lack expression of the estrogen receptor (ER), progesterone receptor (PR), and human epidermal growth factor receptor 2 (HER2). The TNBC may in particular be HER2 negative, such as determined by Fluorescence in situ hybridization (FISH) or determination of protein expression by immunohistochemistry.

The subject may have received at least one prior systemic treatment regimen for locally advanced/metastatic disease to treat said breast cancer, such as at least one prior systemic treatment regimen including anthracycline-, taxane-, antimetabolite- or microtubule inhibitor-containing regimens.

In further embodiments, the subject may have received at the most 4 prior systemic treatment regimens for locally advanced/metastatic disease to treat said breast cancer, such including as at least one prior systemic treatment regimen including anthracycline-, taxane-, antimetabolite- or microtubule inhibitor-containing regimens.

The subject may have received prior treatment with checkpoint inhibitor(s) to treat the breast cancer, such as agent(s) targeting PD-1/PD-L, such as a PD-1/PD-L1 inhibitor; e.g. any one of the PD-/PD-L1 inhibitors listed above.

The subject may have experienced disease progression on or after said prior treatment with checkpoint inhibitor(s) to treat the breast cancer, such as disease progression determined by radiography.

In other embodiments, the subject may be one that has not received prior treatment with checkpoint inhibitor(s) to treat the breast cancer, such a subject that has not received treatment with as agent(s) targeting PD-1/PD-L, such as a PD-1/PD-L1 inhibitor; e.g. the PD-/PD-L1 inhibitors listed above.

The tumor or cancer may be a head and neck cancer, such as a squamous cell carcinoma of the head and neck (SCCHN). Squamous-cell carcinoma of the head and neck (SCCHN) is a major cause of death with over 600,000 cases diagnosed annually worldwide. In 2018, approximately 64,690 people will develop oral cavity, pharyngeal, or laryngeal cancers in the US and an estimated 13,740 deaths will occur over the same period. Head and neck cancers can arise in the oral cavity, pharynx, larynx, nasal cavity, paranasal sinuses, thyroid, and salivary glands. Tobacco use and alcohol greatly increase the risk of developing head and neck cancer. In addition, human papillomavirus (HPV) infection has a causal association with squamous cancers of the oropharynx (particularly tonsils and base of tongue) and recent evidence suggests that HPV may also be associated with increased risk of squamous cell carcinoma of the larynx. Patients with locally HPV-positive head and neck cancers have improved outcomes for response to treatment, PFS, and OS as compared with HPV-negative tumors.

Treatment of head and neck cancers is complex and requires a multidisciplinary approach. The prognosis of patients with recurrent or metastatic SCCHN is generally poor with a median survival of approximately 6 to 12 months depending on patient's performance status and disease-related factors. First-line therapy for fit patients includes cetuximab with cisplatin or carboplatin plus 5-fluorouracil (5-FU). The addition of cetuximab resulted in prolonged survival as compared with platinum and 5-FU alone (10.1 months vs. 7.4 months) as well as prolonged mPFS (3.3 months vs. 5.6 months). Single agent chemotherapy is recommended for patients with poorer performance status. In the past, the most widely used single agents included platinum compounds, taxanes, nab-paclitaxel, methotrexate, fluorouracil, and cetuximab.

In the US and several other countries, pembrolizumab and nivolumab are approved for patients with progressive disease (PD) after platinum-containing chemotherapy. While data from trials exploring the single agent activity of PD-1 targeted appear encouraging, response rates remain low.

In particular, the tumor or cancer may be recurrent of metastatic SCCHN.

In particular embodiments relating to SCCHN, the tumor or cancer is cancer of the oral cavity, pharynx or larynx.

The subject may have received up to four prior systemic treatment regimens for recurrent/metastatic disease to treat the SCCHN and may have experienced disease progression on or after last prior systemic treatment, such as disease progression determined by radiography.

The subject may have received platinum-based chemotherapy to treat the SCCHN, such as treatment with treatment with cisplatin, oxaliplatin, and carboplatin.

Alternatively, the subject may not be eligible for platinum-based therapy and may have received alternative chemotherapy to treat the SCCHN.

The subject may be one that has received prior treatment with checkpoint inhibitor(s) to treat the SCCHN, such as agent(s) targeting PD-1/PD-L, such as a PD-1/PD-L1 inhibitor; e.g. the PD-/PD-L1 inhibitors listed above.

The subject may have experienced disease progression on or after said prior treatment with checkpoint inhibitor(s), such as disease progression determined by radiography.

In other embodiments the subject may be one that has not received prior treatment with checkpoint inhibitor(s), such as agent(s) targeting PD-1/PD-L, such as a PD-1/PD-L1 inhibitor; e.g. a subject that has not received treatment with any of the PD-/PD-L1 inhibitors listed above.

In further embodiments, the tumor or cancer is a cervical cancer. Cervical cancer poses a significant medical problem worldwide with an estimated incidence of more than 500,000 new cases. In the US, approximately 12,800 new cases and 4,210 deaths are estimated to occur in 2017. Cervical cancer has a median age of diagnosis of 49 years in the US and even lower in developing countries. While the 5-year survival rate for patients in the US diagnosed with localized disease is 91%, the prognosis for patients with advanced disease remains poor. Five-year survival rates for advanced/metastatic disease are less than 35%.

First-line treatment for recurrent or metastatic cervical cancer is comprised of bevacizumab combined with paclitaxel and platinum (cisplatin or carboplatin) or paclitaxel and topotecan. Despite a 48% ORR and a median OS of approximately 18 months, almost all patients relapse after this first-line treatment. For second line therapy, pembrolizumab is approved in the US for the treatment of patients with recurrent or metastatic cervical cancer with disease progression on or after chemotherapy and whose tumors express PD-L1 as determined by an FDA-approved test. No additional approved therapies are available however, patients are often treated with single agent modalities including, but not limited to: pemetrexed, topotecan, docetaxel, nab-paclitaxel, vinorelbine, and in some cases bevacizumab. Response rates with single agent treatment are very low (range: 0-15%) and for this reason, cervical cancer remains a population of very high unmet medical need.

The cervical cancer may in particular be of squamous cell, adenocarcinoma or adenosquamous histology.

The subject treated according to the invention may be a subject that has received at least one prior systemic treatment regimen for recurrent/metastatic disease to treat said cervical cancer, such as chemotherapy in combination with treatment targeting vascular endothelial growth factor A, such as treatment with bevacizumab, and has experienced disease progression on or after last prior systemic treatment, such as disease progression determined by radiography.

The subject treated according to the invention may be a subject that has received at the most 4 prior systemic treatment regimens for recurrent/metastatic disease, including chemotherapy in combination with treatment targeting vascular endothelial growth factor A, such as treatment with bevacizumab.

In some embodiments the subject treated according to the invention is a subject that has not received prior treatment with checkpoint inhibitor(s), such as agent(s) targeting PD-1/PD-L, such as a PD-1/PD-L1 inhibitor; e.g. a subject that has not received treatment with any of the PD-/PD-L1 inhibitors listed above.

Preferably, the subject is a female.

A further aspect of the invention provides composition, such as a pharmaceutical composition comprising a taxane chemotherapeutic agent and a binding agent comprising a first binding region binding to human CD137, and a second binding region binding to human PD-L1.

Preferably the amount of binding agent in the composition is about 25-1200 mg or about 1.7×10⁻⁷−8.1×10⁻⁶ mol, such as 25-1200 mg or 1.7×10⁻⁷-8.1×10⁻⁶ mol; about 25-800 mg or about 1.7×10⁻⁷−5.4×10⁻⁶ mol, such as 25-800 mg or 1.7×10⁻⁷−5.4×10⁻⁶ mol or about 25-400 mg or about 1.7×10⁻⁷ −2.7×10⁻⁶ mol, such as 25-400 mg or 1.7×10⁻⁷−2.7×10⁻⁶ mol.

The amount of binding agent administered in said composition may in particular be
about 25-320 mg or about 1.7×10⁻⁷−2.2×10⁻⁶ mol, such as 25-320 mg or 1.7×10⁻⁷−2.2×10⁻⁶ mol;
about 30-320 mg or about 2.4×10⁻⁷−2.2×10⁻⁶ mol; such as 30-320 mg or 2.4×10⁻⁷−2.2×10⁻⁶ mol
about 40-260 mg or about 2.7×10⁻⁷−1.8×10⁻⁶ mol, such as 40-260 mg or 2.7×10⁻⁷−1.8×10⁻⁶ mol;
about 50-200 mg or about 3.4×10⁻⁷−1.4×10⁻⁶ mol, such as 50-200 mg or 3.4×10⁻⁷−1.4×10⁻⁶ mol;
about 60-140 mg or about 4.1×10⁻⁷−9.5×10⁻⁷ mol, such as 60-140 mg or 4.1×10⁻⁷−9.5×10⁻⁷ mol;
about 70-140 mg or about 4.8×10⁻⁷−9.5×10⁻⁷ mol, such as 70-140 mg or 4.8×10⁻⁷−9.5×10⁻⁷ mol;
about 80-120 mg or about 5.5×10⁻⁷−8.2×10⁻⁷ mol, such as 80-120 mg or 5.5×10⁻⁷−8.2×10⁻⁷ mol;
about 90-110 mg or about 6.1×10⁻⁷−7.5×10⁻⁷ mol, such as 90-110 mg or 6.1×10⁻⁷−7.5×10⁻⁷ mol;
about 95-105 mg or about 6.5×10⁻⁷−7.2×10⁻⁷ mol, such as 95-105 mg or 6.5×10⁻⁷−7.2×10⁻⁷ mol;
about 65-120 mg or about 4.4×10⁻⁷−8.2×10⁻⁷ mol, such as 65-120 mg or 4.4×10⁻⁷−8.2×10⁻⁷ mol;
about 70-100 mg or about 4.8×10⁻⁷−6.8×10⁻⁷ mol, such as 70-100 mg or 4.8×10⁻⁷−6.8×10⁻⁷ mol;
or about 75-90 mg or about 5.1×10⁻⁷−6.1×10⁻⁷ mol, such as 75-90 mg or 5.1×10⁻⁷−6.1×10⁻⁷ mol.

The composition or pharmaceutical composition may be formulated with a carrier, excipient and/or diluent as well as any other components suitable for pharmaceutical compositions, including known adjuvants, in accordance with conventional techniques such as those disclosed in Remington: The Science and Practice of Pharmacy, 19^{th} Edition, Gennaro, Ed., Mack Publishing Co., Easton, PA, 1995. The pharmaceutically acceptable carriers or diluents as well as any known adjuvants and excipients should be suitable for the antibody or antibody conjugate of the present invention and the chosen mode of administration. Suitability for carriers and other components of pharmaceutical compositions is determined based on the lack of significant negative impact on the desired biological properties of the chosen compound or pharmaceutical composition of the present invention (e.g., less than a substantial impact [10% or less relative inhibition, 5% or less relative inhibition, etc.] upon antigen binding).

A pharmaceutical composition of the present invention may include diluents, fillers, salts, buffers, detergents (e. g., a nonionic detergent, such as Tween-20 or Tween-80), stabilizers (e.g., sugars or protein-free amino acids), preservatives, solubilizers, and/or other materials suitable for inclusion in a pharmaceutical composition.

Pharmaceutically acceptable carriers include any and all suitable solvents, dispersion media, coatings, antibacterial and antifungal agents, isotonicity agents, antioxidants and absorption-delaying agents, and the like that are physiologically compatible with a compound of the present invention.

Examples of suitable aqueous and non-aqueous carriers which may be employed in the pharmaceutical compositions of the present invention include water, saline, phosphate buffered saline, ethanol, dextrose, polyols (such as glycerol, propylene glycol, polyethylene glycol, and the like), and suitable mixtures thereof, vegetable oils, such as olive oil, corn oil, peanut oil, cottonseed oil, and sesame oil, carboxymethyl cellulose colloidal solutions, tragacanth gum and injectable organic esters, such as ethyl oleate, and/or various buffers. Other carriers are well known in the pharmaceutical arts.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the present invention is contemplated.

Pharmaceutical compositions of the present invention may also comprise pharmaceutically acceptable antioxidants for instance (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

Pharmaceutical compositions of the present invention may also comprise isotonicity agents, such as sugars, polyalcohols, such as mannitol, sorbitol, glycerol or sodium chloride in the compositions.

Pharmaceutical compositions of the present invention may also contain one or more adjuvants appropriate for the chosen route of administration such as preservatives, wetting agents, emulsifying agents, dispersing agents, preservatives or buffers, which may enhance the shelf life or effectiveness of the composition. The combination of compounds of the present invention may be prepared with carriers that will protect the compound against rapid release, such as a controlled release formulation, including implants, transdermal patches, and micro-encapsulated delivery systems. Such carriers may include gelatin, glyceryl monostearate, glyceryl distearate, biodegradable, biocompatible polymers such as ethylene vinyl acetate, polyanhydrides, polyglycolic acid, collagen, poly-ortho esters, and polylactic acid alone or with a wax, or other materials well known in the art. Methods for the preparation of such formulations are generally known to those skilled in the art, see e.g. Sustained and Controlled Release Drug Delivery Systems, J. R. Robinson, ed., Marcel Dekker, Inc., New York, 1978.

In one embodiment, the binding agent used according to the present invention may be formulated to ensure proper distribution in vivo. Pharmaceutically acceptable carriers for parenteral administration include sterile aqueous solutions or dispersions and sterile powders for the extemporaneous preparation of sterile injectable solutions or dispersion. The use of such media and agents for pharmaceutically active substances is known in the art. Except in so far as any conventional media or agent is incompatible with the active compound, use thereof in the compositions of the present invention is contemplated. Other active or therapeutic compounds may also be incorporated into the compositions.

Pharmaceutical compositions for injection must typically be sterile and stable under the conditions of manufacture and storage. The composition may be formulated as a solution, micro-emulsion, liposome, or other ordered structure suitable to high drug concentration. The carrier may be an aqueous or a non-aqueous solvent or dispersion medium containing for instance water, ethanol, polyols (such as glycerol, propylene glycol, polyethylene glycol, and the like), and suitable mixtures thereof, vegetable oils, such as olive oil, and injectable organic esters, such as ethyl oleate. The proper fluidity may be maintained, for example, by the use of a coating such as lecithin, by the maintenance of the required particle size in the case of dispersion and by the use of surfactants. In many cases, it will be preferable to include isotonic agents, for example, sugars, polyalcohols such as glycerol, mannitol, sorbitol, or sodium chloride in the composition. Prolonged absorption of the injectable compositions may be brought about by including in the composition an agent that delays absorption, for example, monostearate salts and gelatin. Sterile injectable solutions may be prepared by incorporating the active compound in the required amount in an appropriate solvent with one or a combination of ingredients e.g. as enumerated above, as required, followed by sterilization microfiltration. Generally, dispersions are prepared by incorporating the active compound into a sterile vehicle that contains a basic dispersion medium and the required other ingredients e.g. from those enumerated above. In the case of sterile powders for the preparation of sterile injectable solutions, examples of methods of preparation are vacuum drying and freeze-drying (lyophilization) that yield a powder of the active ingredient plus any additional desired ingredient from a previously sterile-filtered solution thereof.

Sterile injectable solutions may be prepared by incorporating the active compounds in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, examples of methods of preparation are vacuum-drying and freeze-drying (lyophilization) that yield a powder of the active ingredient plus any additional desired ingredient from a previously sterile-filtered solution thereof.

In one embodiment, the composition according to the invention comprises about 5.5×10⁻⁷ mol or about 80 mg of said binding agent, such as 5.5×10⁻⁷ mol or 80 mg.

In a currently preferred embodiment, the composition according to the invention comprises about 6.8×10⁻⁷ mol or about 100 mg of said binding agent, such as 6.8×10⁻⁷ mol or 100 mg of said binding agent.

In the composition according to the invention the binding agent may be as defined above; e.g. the binding agent may comprise any of the variable regions and constant regions defined above.

In the composition according to the invention, the taxane chemotherapeutic agent is preferably as defined above. The present invention further comprises a dosage unit form of a binding agent or composition as disclosed above Preferably, the dosage unit form is for systemic administration. In particular embodiments, the dosage unit form is for injection or infusion, such as intravenous injection or infusion into a subject.

In the composition or dosage unit form the binding agent is preferably in aqueous solution, such in 0.9% NaCl (saline). The dosage unit form may have a volume of 50-500 mL, such as 50-250 mL, 50-500 mL, 100-500 mL or 100-250 mL.

In yet a further aspect, the present application provides a binding agent comprising a first binding region binding to human CD137, and a second binding region binding to human PD-L1 for use in treatment of cancer or for use in reducing or preventing progression of a tumor, wherein the binding agent is used in combination with a taxane chemotherapeutic agent.

The binding agent for use according to the invention is preferably a binding agent as defined above; e.g. the binding agent may comprise any of the variable regions and constant regions defined above.

In relation to the binding agent for use according to the invention, the taxane chemotherapeutic agent may be as defined above. In currently preferred embodiments, the taxane chemotherapeutic agent is docetaxel.

The binding agent and the taxane chemotherapeutic agent may be in a composition as defined above.

The present invention further provides a taxane chemotherapeutic agent for use in treatment of cancer or for use in reducing or preventing progression of a tumor, wherein the taxane chemotherapeutic agent is used in combination with a binding agent comprising a first binding region binding to human CD137, and a second binding region binding to human PD-L1.

The binding agent may be as defined above; e.g. the binding agent may comprise any of the variable regions and constant regions defined above.

The taxane chemotherapeutic agent may be as defined above. In currently preferred embodiments, the taxane chemotherapeutic drug is docetaxel.

The binding agent and the taxane chemotherapeutic agent may be in a composition as defined above.

Finally, the invention provides a binding agent comprising a first binding region binding to human CD137, and a second binding region binding to human PD-L1 for the manufacture of a medicament for use the treatment of cancer or for reducing or preventing progression of a tumor, wherein the medicament is for use in combination with a taxane chemotherapeutic agent.

The binding agent may be as defined above; e.g. the binding agent may comprise any of the variable regions and constant regions defined above.

The taxane chemotherapeutic agent may be as defined above. In currently preferred embodiments, the taxane chemotherapeutic drug is docetaxel.

The binding agent and the taxane chemotherapeutic agent may be in a composition as defined above.

Additional items of the present disclosure include:
1. A method for reducing or preventing progression of a tumor or treating cancer in a subject, comprising providing to the subject combined treatment with
   I) a binding agent comprising a first binding region binding to human CD137, and a second binding region binding to human PD-L1; and
   ii) a taxane chemotherapeutic agent.
2. The method according to item 1, comprising administering the binding agent and the taxane chemotherapeutic agent to said subject in at least one treatment cycle.
3. The method according to any one of the preceding items, wherein the first binding region binds to human CD137 having the sequence set forth in SEQ ID NO: 24, and/or the second binding region binds to human PD-L1 having the sequence set forth in SEQ ID NO: 26.
4. The method according to any one of the preceding items, wherein the binding agent activates human CD137 when bound thereto and inhibits the binding of human PD-L1 to human PD-1 when bound to PD-L1.
5. The method according to any one of the preceding items, wherein
   a) the first binding region comprises a heavy chain variable region (VH) comprising the CDR1, CDR2, and CDR3 sequences of SEQ ID NO: 1, and a light chain variable region (VL) comprising the CDR1, CDR2, and CDR3 sequences of SEQ ID NO: 5;
   and
   b) the second antigen-binding region comprises a heavy chain variable region (VH) comprising the CDR1, CDR2, and CDR3 sequences of SEQ ID NO: 8, and a light chain variable region (VL) comprising the CDR1, CDR2, and CDR3 sequences of SEQ ID NO: 12.
6. The method according to any one of the preceding items, wherein
   a) the first binding region comprises a heavy chain variable region (VH) comprising the CDR1, CDR2, and CDR3 sequences set forth in: SEQ ID NO: 2, 3, and 4, respectively, and a light chain variable region (VL) comprising the CDR1, CDR2, and CDR3 sequences set forth in: SEQ ID NO: 6, GAS, 7, respectively;
   and
   b) the second antigen-binding region comprises a heavy chain variable region (VH) comprising the CDR1, CDR2, and CDR3 sequences set forth in: SEQ ID NO: 9, 10, 11 respectively, and a light chain variable region (VL) comprising the CDR1, CDR2, and CDR3 sequences set forth in: SEQ ID NO: 13, DDN, 14, respectively.
7. The method according to any one of the preceding items, wherein
   a) The first binding region comprises a heavy chain variable region (VH) comprising an amino acid sequence having at least 90%, at least 95%, at least 97%, at least 99%, or 100% sequence identity to SEQ ID NO: 1 and a light chain variable region (VL) region comprising an amino acid sequence having at least 90%, at least 95%, at least 97%, at least 99%, or 100% sequence identity to SEQ ID NO: 5;
   and
   b) the second binding region comprises a heavy chain variable region (VH) comprising an amino acid sequence having at least 90%, at least 95%, at least 97%, at least 99%, or 100% sequence identity to SEQ ID NO: 8 and a light chain variable region (VL) region comprising an amino acid sequence having at least 90%, at least 95%, at least 97%, at least 99%, or 100% sequence identity to SEQ ID NO: 12.
8. The method according to any one of the preceding items, wherein
   a) The first binding region comprises a heavy chain variable region (VH) comprising the amino acid sequence set forth in SEQ ID NO: 1 and a light chain variable region (VL) region comprising the amino acid sequence set forth in SEQ ID NO: 5;
   and
   b) the second binding region comprises a heavy chain variable region (VH) comprising the amino acid sequence set forth in SEQ ID NO: 8 and a light chain variable region (VL) region comprising the amino acid sequence set forth in SEQ ID NO: 12.
9. The method according to any one of the preceding items, wherein the binding agent is an antibody, a multispecific antibody, such as a bispecific antibody.
10. The method according to any one of the preceding items, wherein the binding agent is in the format of a full-length antibody or an antibody fragment.
11. The method according to any one of items 5-10, wherein each variable region comprises three complementarity determining regions (CDR1, CDR2, and CDR3) and four framework regions (FR1, FR2, FR3, and FR4).
12. The method according to item 11, wherein said complementarity determining regions and said framework regions are arranged from amino-terminus to carboxy-terminus in the following order: FR1, CDR1, FR2, CDR2, FR3, CDR3, FR4.
13. The method according to any one of the preceding items, which comprises
   i) a polypeptide comprising, consisting of or consisting essentially of, said first heavy chain variable region (VH) and a first heavy chain constant region (CH), and
   ii) a polypeptide comprising, consisting of or consisting essentially of, said second heavy chain variable region (VH) and a second heavy chain constant region (CH).
14. The method according to any one of the preceding items, which comprises
   i) a polypeptide comprising said first light chain variable region (VL) and further comprising a first light chain constant region (CL), and
   ii) a polypeptide comprising said second light chain variable region (VL) and further comprising a second light chain constant region (CL).
15. The method according to any one of the preceding items, wherein the binding agent is an antibody comprising a first binding arm and a second binding arm, wherein the first binding arm comprises
   i) a polypeptide comprising said first heavy chain variable region (VH) and said first heavy chain constant region (CH), and
   ii) a polypeptide comprising said first light chain variable region (VL) and said first light chain constant region (CL);
   and the second binding arm comprises
   iii) a polypeptide comprising said second heavy chain variable region (VH) and said second heavy chain constant region (CH), and
   iv) a polypeptide comprising said second light chain variable region (VL) and said second light chain constant region (CL).
16. The method according to any one of the preceding items, which comprises
   i) a first heavy chain and light chain comprising said antigen-binding region capable of binding to CD137, and
   ii) a second heavy chain and light chain comprising said antigen-binding region capable of binding PD-L1.
17. The method according to any one of the preceding items, wherein said binding agent comprises
   i) a first heavy chain and light chain comprising said antigen-binding region capable of binding to CD137, the first heavy chain comprising a first heavy chain constant region and the first light chain comprising a first light chain constant region; and
   ii) a second heavy chain and light chain comprising said antigen-binding region capable of binding PD-L1, the second heavy chain comprising a second heavy chain constant region and the second light chain comprising a second light chain constant region.
18. The method according to any one of items 13-17, wherein each of the first and second heavy chain constant regions (CH) comprises one or more of a constant heavy chain 1 (CH1) region, a hinge region, a constant heavy chain 2 (CH2) region and a constant heavy chain 3 (CH3) region, preferably at least a hinge region, a CH2 region and a CH3 region.
19. The method according to any one of items 13-18, wherein each of the first and second heavy chain constant regions (CHs) comprises a CH3 region and wherein the two CH3 regions comprise asymmetrical mutations.
20. The method according to any one of items 13-19, wherein in said first heavy chain constant region (CH) at least one of the amino acids in a position corresponding to a position selected from the group consisting of T366, L368, K370, D399, F405, Y407, and K409 in a human IgG1 heavy chain according to EU numbering has been substituted, and in said second heavy chain constant region (CH) at least one of the amino acids in a position corresponding to a position selected from the group consisting of T366, L368, K370, D399, F405, Y407, and K409 in a human IgG1 heavy chain according to EU numbering has been substituted, and wherein said first and said second heavy chains are not substituted in the same positions.
21. The method according to item 20, wherein (i) the amino acid in the position corresponding to F405 in a human IgG1 heavy chain according to EU numbering is L in said first heavy chain constant region (CH), and the amino acid in the position corresponding to K409 in a human IgG1 heavy chain according to EU numbering is R in said second heavy chain constant region (CH), or (ii) the amino acid in the position corresponding to K409 in a human IgG1 heavy chain according to EU numbering is R in said first heavy chain, and the amino acid in the position corresponding to F405 in a human IgG1 heavy chain according to EU numbering is L in said second heavy chain.
22. The method according to any of the preceding items, wherein said binding agent induces Fc-mediated effector function to a lesser extent compared to another antibody comprising the same first and second antigen binding regions and two heavy chain constant regions (CHs) comprising human IgG1 hinge, CH2 and CH3 regions.
23. The method according to item 22, wherein said first and second heavy chain constant regions (CHs) are modified so that the antibody induces Fc-mediated effector function to a lesser extent compared to an antibody which is identical except for comprising non-modified first and second heavy chain constant regions (CHs).
24. The method according to item 23, wherein each of said non-modified first and second heavy chain constant regions (CHs) comprises the amino acid sequence set forth in SEQ ID NO: 15.
25. The method according to any of items 23-24, wherein said Fc-mediated effector function is measured by binding to Fcγ receptors, binding to C1q, or induction of Fc-mediated cross-linking of Fcγ receptors.
26. The method according to item 25, wherein said Fc-mediated effector function is measured by binding to C1q.
27. The method according to any one of items 22-26, wherein said first and second heavy chain constant regions have been modified so that binding of C1q to said antibody is reduced compared to a wild-type antibody, preferably reduced by at least 70%, at least 80%, at least 90%, at least 95%, at least 97%, or 100%, wherein C1q binding is preferably determined by ELISA.
28. The method according to any one of the preceding items, wherein in at least one of said first and second heavy chain constant regions (CH), one or more amino acids in the positions corresponding to positions L234, L235, D265, N297, and P331 in a human IgG1 heavy chain according to EU numbering, are not L, L, D, N, and P, respectively.
29. The method according to item 28, wherein the positions corresponding to positions L234 and L235 in a human IgG1 heavy chain according to EU numbering are F and E, respectively, in said first and second heavy chains.
30. The method according to item 28 or 29, wherein the positions corresponding to positions L234, L235, and D265 in a human IgG1 heavy chain according to EU numbering are F, E, and A, respectively, in said first and second heavy chain constant regions (HCs).
31. The method according to any one of items 28-30, wherein the positions corresponding to positions L234 and L235 in a human IgG1 heavy chain according to EU numbering of both the first and second heavy chain constant regions are F and E, respectively, and wherein (i) the position corresponding to F405 in a human IgG1 heavy chain according to EU numbering of the first heavy chain constant region is L, and the position corresponding to K409 in a human IgG1 heavy chain according to EU numbering of the second heavy chain is R, or (ii) the position corresponding to K409 in a human IgG1 heavy chain according to EU numbering of the first heavy chain constant region is R, and the position corresponding to F405 in a human IgG1 heavy chain according to EU numbering of the second heavy chain is L.
32. The method according to any one of items 28-31, wherein the positions corresponding to positions L234, L235, and D265 in a human IgG1 heavy chain according to EU numbering of both the first and second heavy chain constant regions are F, E, and A, respectively, and wherein (i) the position corresponding to F405 in a human IgG1 heavy chain according to EU numbering of the first heavy chain constant region is L, and the position corresponding to K409 in a human IgG1 heavy chain according to EU numbering of the second heavy chain constant region is R, or (ii) the position corresponding to K409 in a human IgG1 heavy chain according to EU numbering of the first heavy chain is R, and the position corresponding to F405 in a human IgG1 heavy chain according to EU numbering of the second heavy chain is L.
33. The method according to any one of items 13-32, wherein the constant region of said first and/or second heavy chain comprises or consists essentially of or consists of an amino acid sequence selected from the group consisting of
   a) the sequence set forth in SEQ ID NO: 15 [IgG1-FC],
   b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
   c) a sequence having at the most 10 substitutions, such as at the most 9 substitutions, at the most 8, at the most 7, at the most 6, at the most 5, at the most 4, at the most 3, at the most 2 or at the most 1 substitution compared to the amino acid sequence defined in a) or b).
34. The method according to any one of items 13-33, wherein the constant region of said first or second heavy chain, such as the second heavy chain, comprises or consists essentially of or consists of an amino acid sequence selected from the group consisting of
   a) the sequence set forth in SEQ ID NO: 16 [IgG1-F405L],
   b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
   c) a sequence having at the most 9 substitutions, such as at the most 8, at the most 7, at the most 6, at the most 5, at the most 4, at the most 3, at the most 2 or at the most 1 substitution compared to the amino acid sequence defined in a) or b).
35. The method according to any one of items 13-34, wherein the constant region of said first or second heavy chain, such as the first heavy chain comprises or consists essentially of or consists of an amino acid sequence selected from the group consisting of
   a) the sequence set forth in SEQ ID NO: 17 [IgG1-F409R]
   b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
   c) a sequence having at the most 10 substitutions, such as at the most 9 substitutions, at the most 8, at the most 7, at the most 6, at the most 5, at the most 4 substitutions, at the most 3, at the most 2 or at the most 1 substitution compared to the amino acid sequence defined in a) or b).
36. The method according to any one of items 13-35, wherein the constant region of said first and/or second heavy chain comprises or consists essentially of or consists of an amino acid sequence selected from the group consisting of
   a) the sequence set forth in SEQ ID NO: 18 [IgG1-Fc_FEA],
   b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
   c) a sequence having at the most 7 substitutions, such as at the most 6 substitutions, at the most 5, at the most 4, at the most 3, at the most 2 or at the most 1 substitution compared to the amino acid sequence defined in a) or b).
37. The method according to any one of items 13-36, wherein the constant region of said first and/or second heavy chain, such as the second heavy chain, comprises or consists essentially of or consists of an amino acid sequence selected from the group consisting of
   a) the sequence set forth in SEQ ID NO: 20 [IgG1-Fc_FEAL],
   b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
   c) a sequence having at the most 6 substitutions, such as at the most 5 substitutions, at the most 4 substitutions, at the most 3, at the most 2 or at the most 1 substitution compared to the amino acid sequence defined in a) or b).
38. The method according to any one of items 13-37, wherein the constant region of said first and/or second heavy chain, such as the first heavy chain, comprises or consists essentially of or consists of an amino acid sequence selected from the group consisting of
   a) the sequence set forth in SEQ ID NO: 19 [IgG1-Fc_FEAR]
   b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
   c) a sequence having at the most 6 substitutions, such as at the most 5 substitutions, at the most 4, at the most 3, at the most 2 or at the most 1 substitution compared to the amino acid sequence defined in a) or b).
39. The method according to any one of the preceding items, wherein said binding agent comprises a kappa (κ) light chain constant region.
40. The method according to any one of the preceding items, wherein said binding agent comprises a lambda (λ) light chain constant region.
41. The method according to any one of the preceding items, wherein said first light chain constant region is a kappa (κ) light chain constant region.
42. The method according to any one of the preceding items, wherein said second light chain constant region is a lambda (λ) light chain constant region.
43. The method according to any one of the preceding items, wherein said first light chain constant region is a lambda (λ) light chain constant region.
44. The method according to any one of the preceding items, wherein said second light chain constant region is a kappa (κ) light chain constant region.
45. The method according to any one of items 39-44, wherein the kappa (κ) light chain comprises an amino acid sequence selected from the group consisting of
   a) the sequence set forth in SEQ ID NO: 21,
   b) a subsequence of the sequence in a), such as a subsequence wherein 1, 2, 3, 4, 5, 6, 7, 8, 9 or 10 consecutive amino acids has/have been deleted, starting from the N-terminus or C-terminus of the sequence defined in a); and
   c) a sequence having at the most 10 substitutions, such as at the most 9 substitutions, at the most 8, at the most 7, at the most 6, at the most 5, at the most 4 substitutions, at the most 3, at the most 2 or at the most 1 substitution, compared to the amino acid sequence defined in a) or b).
46. The method according to any one of items 40-45, wherein the lambda (λ) light chain comprises an amino acid sequence selected from the group consisting of
   a) the sequence set forth in SEQ ID NO: 22,
   b) a subsequence of the sequence in a), such as a subsequence wherein 1, 2, 3, 4, 5, 6, 7, 8, 9 or 10 consecutive amino acids has/have been deleted, starting from the N-terminus or C-terminus of the sequence defined in a); and
   c) a sequence having at the most 10 substitutions, such as at the most 9 substitutions, at the most 8, at the most 7, at the most 6, at the most 5, at the most 4 substitutions, at the most 3, at the most 2 or at the most 1 substitution, compared to the amino acid sequence defined in a) or b).
47. The method according to any one of the preceding items, wherein the binding agent is of an isotype selected from the group consisting of IgG1, IgG2, IgG3, and IgG4.
48. The method according to any one of the preceding items, wherein the binding agent is a full-length IgG1 antibody.
49. The method according to any one of the preceding items, wherein said antibody is of the IgG1m(f) allotype.
50. The method according to any one of the preceding items, wherein said antibody is acasunlimab or a biosimilar thereof.
51. The method according to any one of the preceding items, wherein the taxane is selected from the group consisting of: Docetaxel, paclitaxel, cabazitaxel and tesetaxel.
52. The method according to any one of the preceding items, wherein the taxane chemotherapeutic drug is docetaxel.
53. The method according to any one of the preceding items, wherein the amount of binding agent administered in each dose and/or in each treatment cycle is
   a) about 0.3-15 mg/kg body weight or about 25-1200 mg in total; and/or
   b) about 2.1×10⁻⁹−1.2×10⁻⁷ mol/kg body weight or about 1.7×10⁻⁷−8.1×10⁻⁶ mol in total.
54. The method according to any one of the preceding items, wherein the amount of binding agent administered in each dose and/or in each treatment cycle is
   a) about 0.3-10 mg/kg body weight or about 25-800 mg in total; and/or
   b) about 2.1×10⁻⁹−6.8×10⁻⁸ mol/kg body weight or about 1.7×10⁻⁷−5.4×10⁻⁶ mol in total.
55. The method according to any one of the preceding items, wherein the amount of binding agent administered in each dose and/or in each treatment cycle is
   a) about 0.3-5 mg/kg body weight or about 25-400 mg in total; and/or
   b) about 2.1×10⁻⁹−3.4×10⁻⁸ mol/kg body weight or about 1.7×10⁻⁷−2.7×10⁻⁶ mol in total.
56. The method according to any one of the preceding items, wherein the amount of binding agent administered in each dose and/or in each treatment cycle is
   a) about 1.25 mg/kg body weight or about 100 mg in total; and/or
   b) about 8.5×10⁻⁹ mol/kg body weight or about 6.8×10⁻⁷ mol in total.
57. The method according to any one of the preceding items, wherein the amount of taxane chemotherapeutic agent administered in each dose and/or in each treatment cycle is about 10-200 mg/m², such as 20-40 mg/m², 30-50 mg/m², 40-100 mg/m², 50-100 mg/m², 50-80 mg/m², 50-70 mg/m², 50-60 mg/m², 50-110 mg/m², 60-100 mg/m², 60-100 mg/m², 60-90 mg/m², 70-80 mg/m², 80-200 mg/m², 90-180 mg/m², 90-110 mg/m², 100-175 mg/m², or such as a bout 170-180 mg/m².
58. The method according to any one of the preceding items, wherein the taxane chemotherapeutic agent is docetaxel and the amount administered in each dose and/or in each treatment cycle is about 50-60 mg/m², such as about 55 mg/m².
59. The method according to any one of the preceding items, wherein the taxane chemotherapeutic agent is docetaxel and the amount administered in each dose and/or in each treatment cycle is about 70-80 mg/m², such as about 75 mg/m².
60. The method according to any one of the preceding items, wherein the binding agent and/or the taxane chemotherapeutic agent is/are administered by systemic administration.
61. The method according to any one of the preceding items, wherein the binding agent and/or the taxane chemotherapeutic agent is/are administered by intravenous injection or infusion.
62. The method according to any one of the preceding items, wherein at least one dose of said binding agent and at least one dose of said taxane chemotherapeutic drug are administered in each treatment cycle.
63. The method according to any one of the preceding items, wherein each treatment cycle is three weeks (21 days).
64. The method according to any one of the preceding items, wherein said binding agent and said taxane chemotherapeutic agent are administered on the same day.
65. The method according to any one of the preceding items, wherein each dose of said binding agent is infused over a minimum of 30 minutes, such as over a minimum of 60 minutes, a minimum of 90 minutes, a minimum of 120 minutes or a minimum of 240 minutes.
66. The method according to any one of the preceding items, wherein administration of the binding agent precedes administration of the taxane chemotherapeutic agent by at least 30 minutes, such as by at least 1 hour or such as at least 2 hours.
67. The method according to any one of the preceding items, wherein one dose of said binding agent and/or one dose of said taxane chemotherapeutic agent is/are administered on day 1 of each treatment cycle.
68. The method according to any one of the preceding items, wherein one dose of said binding agent is administered every third week (1Q3W), such as on day 1 on each three-week treatment cycle.
69. The method according to any one of the preceding items, wherein one dose of said taxane chemotherapeutic agent is administered every third week (1Q3W), such as on day 1 on each three-week treatment cycle.
70. The method according to any one of the preceding items, wherein each dose of taxane chemotherapeutic agent is preceded by steroid premedication, such as premedication with an oral corticosteroid; e.g. administration of about 8 mg dexamethasone 2 times a day for 3 days starting 1 day prior to administration of the taxane chemotherapeutic agent.
71. The method according to any one of the preceding items, wherein the subject is a human subject.
72. The method according to any one of the preceding items, wherein the tumor or cancer is a solid tumor.
73. The method according to any one of the preceding items, wherein the tumor or cancer is selected from the group consisting of melanoma, ovarian cancer, lung cancer (e.g. non-small cell lung cancer (NSCLC), colorectal cancer, head and neck cancer, gastric cancer, breast cancer, renal cancer, urothelial cancer, bladder cancer, esophageal cancer, pancreatic cancer, hepatic cancer, thymoma and thymic carcinoma, brain cancer, glioma, adrenocortical carcinoma, thyroid cancer, other skin cancers, sarcoma, multiple myeloma, leukemia, lymphoma, myelodysplastic syndromes, ovarian cancer, endometrial cancer, prostate cancer, penile cancer, cervical cancer, Hodgkin's lymphoma, non-Hodgkin's lymphoma, Merkel cell carcinoma and mesothelioma.
74. The method according to any one of the preceding items, wherein the tumor or cancer is selected from the group consisting of lung cancer (e.g. non-small cell lung cancer (NSCLC), urothelial cancer (cancer of the bladder, ureter, urethra, or renal pelvis), endometrial cancer (EC), breast cancer (e.g. triple negative breast cancer (TNBC)), squamous cell carcinoma of the head and neck (SCCHN) (e.g. cancer of the oral cavity, pharynx or larynx) and cervical cancer.
75. The method according to any one of the preceding items, wherein the tumor or cancer is a lung cancer.
76. The method according to item 75, wherein the lung cancer is a non-small cell lung cancer (NSCLC), such as a squamous or non-squamous NSCLC.
77. The method according to item 56, wherein the NSCLC does not have an epidermal growth factor (EGFR)-sensitizing mutation and/or anaplastic lymphoma (ALK) translocation/ROS1 rearrangement.
78. The method according to any one of items 75-77, wherein the subject has received up to four prior systemic treatment regimens for advanced/metastatic disease and has experienced disease progression on or after last prior systemic treatment, such as disease progression determined by radiography.
79. The method according to item 78, wherein the subject has received platinum-based chemotherapy.
80. The method according to item 78, wherein the subject is not eligible for platinum-based therapy and has received alternative chemotherapy, e.g., a treatment with gemcitabine-containing regimen.
81. The method according to any one of the preceding items, wherein the subject has received prior treatment with checkpoint inhibitor(s), such as agent(s) targeting PD-1/PD-L, such as a PD-1/PD-L1 inhibitor.
82. The method according to any one of the preceding items, wherein the subject has experienced disease progression on or after treatment with checkpoint inhibitor(s), such as agent(s) targeting PD-1/PD-L, such as a PD-1/PD-L1 inhibitor.
83. The method according to any one of the preceding items, wherein the subject has experienced disease progression on or after last prior treatment with checkpoint inhibitor(s), such as agent(s) targeting PD-1/PD-L, such as a PD-1/PD-L1 inhibitor.
84. The method according to any one of item 78-83, wherein the subject has experienced disease progression on or after last prior systemic treatment, such as disease progression determined by radiography.
85. The method according to any one of the preceding items, wherein the subject has not received prior treatment with checkpoint inhibitor(s), such as agent(s) targeting PD-1/PD-L, such as a PD-1/PD-L1 inhibitor.
86. The method according to any one of the preceding items, wherein the tumor or cancer is an endometrial cancer.
87. The method according to item 86, wherein the subject has epithelial endometrial histology including: endometrioid, serous, squamous, clear-cell carcinoma, or carcinosarcoma.
88. The method according to item 86 or 87, wherein the subject has received up to four prior systemic treatment regimens for advanced/metastatic disease and has experienced disease progression on or after last prior systemic treatment, such as disease progression determined by radiography.
89. The method according to any one of items 86-88, wherein the subject has not received prior treatment with checkpoint inhibitor(s), such as agent(s) targeting PD-1/PD-L, such as a PD-1/PD-L1 inhibitor.
90. The method according to any one of the preceding items, wherein the tumor or cancer is an urothelial cancer, including cancer of the bladder, ureter, urethra, or renal pelvis.
91. The method according to item 90, wherein the subject has received up to four prior systemic treatment regimens for advanced/metastatic disease and has experienced disease progression on or after last prior systemic treatment, such as disease progression determined by radiography.
92. The method according to item 90 or 91, wherein the subject has received prior treatment with checkpoint inhibitor(s), such as agent(s) targeting PD-1/PD-L, such as a PD-1/PD-L1 inhibitor.
93. The method according to item 90 or 91, wherein the subject has received platinum-based chemotherapy.
94. The method according to any one of items 90 or 91, wherein the subject is not eligible for platinum-based therapy and has received alternative chemotherapy, e.g., a treatment with gemcitabine-containing regimen.
95. The method according to any one of the preceding items, wherein the tumor or cancer is a breast cancer, such as a triple negative breast cancer (TNBC).
96. The method according to item 95, wherein the TNBC is HER2 negative, such as determined by Fluorescence in situ hybridization (FISH) or determination of protein expression by immunohistochemistry. Progesterone receptor negative, estrogen receptor negative. . .
97. The method according to item 95 or 76, wherein the subject has received at least one prior systemic treatment regimen for locally advanced/metastatic disease, such as at least one prior systemic treatment regimen including anthracycline-, taxane-, antimetabolite- or microtubule inhibitor-containing regimens.
98. The method according to item 97, wherein the subject has received at the most 4 prior systemic treatment regimens for locally advanced/metastatic disease, such including as at least one prior systemic treatment regimen including anthracycline-, taxane-, antimetabolite- or microtubule inhibitor-containing regimens.
99. The method according to any one of items 95-98, wherein the subject has received prior treatment with checkpoint inhibitor(s), such as agent(s) targeting PD-1/PD-L, such as a PD-1/PD-L1 inhibitor.
100. The method according to item 99, wherein the subject has experienced disease progression on or after said prior treatment with checkpoint inhibitor(s), such as disease progression determined by radiography.
101. The method according to any one of items 95-98, wherein the subject has not received prior treatment with checkpoint inhibitor(s), such as agent(s) targeting PD-1/PD-L, such as a PD-1/PD-L1 inhibitor.
102. The method according to any one of the preceding items, wherein the tumor or cancer is a head and neck cancer, such as a squamous cell carcinoma of the head and neck (SCCHN).
103. The method according to item 102, wherein the tumor or cancer is recurrent or metastatic SCCHN.
104. The method according to item 102 or 103, wherein the tumor or cancer is cancer of the oral cavity, pharynx or larynx.
105. The method according to any one of items 102-104, wherein the subject has received up to four prior systemic treatment regimens for recurrent/metastatic disease and has experienced disease progression on or after last prior systemic treatment, such as disease progression determined by radiography.
106. The method according to item 105, wherein the subject has received platinum-based chemotherapy.
107. The method according to item 105, wherein the subject is not eligible for platinum-based therapy and has alternative chemotherapy.
108. The method according to any one of items 102-107, wherein the subject has received prior treatment with checkpoint inhibitor(s), such as agent(s) targeting PD-1/PD-L, such as a PD-1/PD-L1 inhibitor.
109. The method according to item 108, wherein the subject has experienced disease progression on or after said prior treatment with checkpoint inhibitor(s), such as disease progression determined by radiography.
110. The method according to any one of items 102-107, wherein the subject has not received prior treatment with checkpoint inhibitor(s), such as agent(s) targeting PD-1/PD-L, such as a PD-1/PD-L1 inhibitor.
111. The method according to any one of the preceding items, wherein the tumor or cancer is a cervical cancer.
112. The method according to item 111, wherein the cervical cancer is of squamous cell, adenocarcinoma or adenosquamous histology.
113. The method according to item 111 or 112, wherein the subject has received at least one prior systemic treatment regimen for recurrent/metastatic disease, such as chemotherapy in combination with treatment targeting vascular endothelial growth factor A, such as treatment with bevacizumab, and has experienced disease progression on or after last prior systemic treatment, such as disease progression determined by radiography.
114. The method according to item 113, wherein the subject has received at the most 4 prior systemic treatment regimens for recurrent/metastatic disease, including chemotherapy in combination with treatment targeting vascular endothelial growth factor A, such as treatment with bevacizumab.
115. The method according to any one of item 111-114, wherein the subject has not received prior treatment with checkpoint inhibitor(s), such as agent(s) targeting PD-1/PD-L, such as a PD-1/PD-L1 inhibitor.
116. A composition comprising a taxane chemotherapeutic agent and a binding agent comprising a first binding region binding to human CD137, and a second binding region binding to human PD-L1.
117. The composition according to item 116, wherein the amount of binding agent in the composition is between 25-400 mg or 1.7×10⁻⁷-2.7×10⁻⁶ mol.
118. The composition according to item 116, comprising about 80 mg of said binding agent.
119. The composition according to any one of items 116-118, wherein the binding agent is as defined in any one of items 3-50.
120. The composition according to any one of items 116-119, wherein the taxane chemotherapeutic drug is as defined in any one of items 51-52.
121. The composition according to any one of items 116-120, wherein the composition is for systemic administration.
122. The composition according to any one of items 116-121, wherein the composition is for injection or infusion, such as intravenous injection or infusion.
123. The composition according to any one of items 122, wherein the binding agent and the taxane chemotherapeutic drug are in aqueous solution, such in 0.9% NaCl (saline), at a volume of 50-500 mL, such as 100-250 mL.
124. A binding agent comprising a first binding region binding to human CD137, and a second binding region binding to human PD-L1 for use in treatment of cancer or for use in reducing or preventing progression of a tumor, wherein the binding agent is used in combination with a taxane chemotherapeutic agent.
125. The binding agent for use according to item 124, the binding agent being as defined in any one of items 3-50.
126. The binding agent for use according to item 124 or 125, wherein the taxane chemotherapeutic agent is as defined in any one of items 51-52.
127. A taxane chemotherapeutic agent for use in treatment of cancer or for use in reducing or preventing progression of a tumor, wherein the taxane chemotherapeutic agent is used in combination with a binding agent comprising a first binding region binding to human CD137, and a second binding region binding to human PD-L1.
128. The taxane chemotherapeutic agent for use according to item 127, wherein the binding agent is as defined in any one of items 3-50.
129. The taxane chemotherapeutic agent for use according to item 127 or 128, the taxane chemotherapeutic agent being as defined in any one of items

### Sequences

TABLE 7

| SEQ ID | NAME | SEQUENCE |
|---|---|---|
| 1 | VH_CD137-009-H7 | EVQLVESGGGLVQPGRSLRLSCTASGFSLNDYWMSWVRQAPGKGLEWV |
| | | GYIDVGGSLYYAASVKGRFTISRDDSKSIAYLQMNSLKTEDTAVYYCARGGL |
| | | TYGFDLWGQGTLVTVSS |
| 2 | VH_CD137-009-H7_CDR1 | GFSLNDYW |
| 3 | VH_CD137-009-H7_CDR2 | IDVGGSL |
| 4 | VH_CD137-009-H7_CDR3 | ARGGLTYGFDL |
| 5 | VL_CD137-009-L2 | DIVMTQSPSSLSASVGDRVTITCQASEDISSYLAWYQQKPGKAPKRLIYGAS |
| | | DLASGVPSRFSASGSGTDYTFTISSLQPEDIATYYCHYYATISGLGVAFGGGT |
| | | KVEIK |
| 6 | VL_CD137-009-L2_CDR1 | EDISSY |
| | VL_CD137-009-L2_CDR2 | GAS |
| 7 | VL_CD137-009-L2_CDR3 | HYYATISGLGVA |
| 8 | VH-PD-L1-547 | EVQLLEPGGGLVQPGGSLRLSCEASGSTFSTYAMSWVRQAPGKGLEWVS |
| | | GFSGSGGFTFYADSVRGRFTISRDSSKNTLFLQMSSLRAEDTAVYYCAIPAR |
| | | GYNYGSFQHWGQGTLVTVSS |
| 9 | VH- PD-L1-547-CDR1 | GSTFSTYA |
| 10 | VH- PD-L1-547-CDR2 | FSGSGGFT |
| 11 | VH- PD-L1-547-CDR3 | AIPARGYNYGSFQH |
| 12 | VL- PD-L1-547 | SYVLTQPPSVSVAPGQTARITCGGNNIGSKSVHWYQQKPGQAPVLVVYD |
| | | DNDRPSGLPERFSGSNSGNTATLTISRVEAGDEADYYCQVWDSSSDHVVF |
| | | GGGTKLTVL |
| 13 | VL- PD-L1-547-CDR1 | NIGSKS |
| | VL- PD-L1-547-CDR2 | DDN |
| 14 | VL- PD-L1-547-CDR3 | QVWDSSSDHVV |
| 15 | IgG1-Fc | ASTKGPSVFPLAPSSKSTSGGTAALGCLVKDYFPEPVTVSWNSGALTSGVH |
| | | TFPAVLQSSGLYSLSSVVTVPSSSLGTQTYICNVNHKPSNTKVDKRVEPKSC |
| | | DKTHTCPPCPAPELLGGPSVFLFPPKPKDTLMISRTPEVTCVVVDVSHEDPE |
| | | VKFNWYVDGVEVHNAKTKPREEQYNSTYRVVSVLTVLHQDWLNGKEYKC |
| | | KVSNKALPAPIEKTISKAKGQPREPQVYTLPPSREEMTKNQVSLTCLVKGFY |
| | | PSDIAVEWESNGQPENNYKTTPPVLDSDGSFFLYSKLTVDKSRWQQGNVF |
| | | SCSVMHEALHNHYTQKSLSLSPG |
| 16 | IgG1-Fc_F405L | ASTKGPSVFPLAPSSKSTSGGTAALGCLVKDYFPEPVTVSWNSGALTSGVH |
| | | TFPAVLQSSGLYSLSSVVTVPSSSLGTQTYICNVNHKPSNTKVDKRVEPKSC |
| | | DKTHTCPPCPAPELLGGPSVFLFPPKPKDTLMISRTPEVTCVVVDVSHEDPE |
| | | VKFNWYVDGVEVHNAKTKPREEQYNSTYRVVSVLTVLHQDWLNGKEYKC |
| | | KVSNKALPAPIEKTISKAKGQPREPQVYTLPPSREEMTKNQVSLTCLVKGFY |
| | | PSDIAVEWESNGQPENNYKTTPPVLDSDGSFLLYSKLTVDKSRWQQGNVF |
| | | SCSVMHEALHNHYTQKSLSLSPG |
| 17 | IgG1-Fc_K409R | ASTKGPSVFPLAPSSKSTSGGTAALGCLVKDYFPEPVTVSWNSGALTSGVH |
| | | TFPAVLQSSGLYSLSSVVTVPSSSLGTQTYICNVNHKPSNTKVDKRVEPKSC |
| | | DKTHTCPPCPAPELLGGPSVFLFPPKPKDTLMISRTPEVTCVVVDVSHEDPE |
| | | VKFNWYVDGVEVHNAKTKPREEQYNSTYRVVSVLTVLHQDWLNGKEYKC |
| | | KVSNKALPAPIEKTISKAKGQPREPQVYTLPPSREEMTKNQVSLTCLVKGFY |
| | | PSDIAVEWESNGQPENNYKTTPPVLDSDGSFFLYSRLTVDKSRWQQGNVF |
| | | SCSVMHEALHNHYTQKSLSLSPG |
| 18 | IgG1-Fc_FEA | ASTKGPSVFPLAPSSKSTSGGTAALGCLVKDYFPEPVTVSWNSGALTSGVH |
| | | TFPAVLQSSGLYSLSSVVTVPSSSLGTQTYICNVNHKPSNTKVDKRVEPKSC |
| | | DKTHTCPPCPAPEFEGGPSVFLFPPKPKDTLMISRTPEVTCVVVAVSHEDP |
| | | EVKFNWYVDGVEVHNAKTKPREEQYNSTYRVVSVLTVLHQDWLNGKEYK |
| | | CKVSNKALPAPIEKTISKAKGQPREPQVYTLPPSREEMTKNQVSLTCLVKGF |
| | | YPSDIAVEWESNGQPENNYKTTPPVLDSDGSFFLYSKLTVDKSRWQQGNV |
| | | FSCSVMHEALHNHYTQKSLSLSPG |
| 19 | IgG1-FEAR-Fc | ASTKGPSVFPLAPSSKSTSGGTAALGCLVKDYFPEPVTVSWNSGALTSGVH |
| | | TFPAVLQSSGLYSLSSVVTVPSSSLGTQTYICNVNHKPSNTKVDKRVEPKSC |
| | | DKTHTCPPCPAPEFEGGPSVFLFPPKPKDTLMISRTPEVTCVVVAVSHEDP |
| | | EVKFNWYVDGVEVHNAKTKPREEQYNSTYRVVSVLTVLHQDWLNGKEYK |
| | | CKVSNKALPAPIEKTISKAKGQPREPQVYTLPPSREEMTKNQVSLTCLVKGF |
| | | YPSDIAVEWESNGQPENNYKTTPPVLDSDGSFFLYSRLTVDKSRWQQGN |
| | | VFSCSVMHEALHNHYTQKSLSLSPG |
| 20 | IgG1-FEAL-Fc | ASTKGPSVFPLAPSSKSTSGGTAALGCLVKDYFPEPVTVSWNSGALTSGVH |
| | | TFPAVLQSSGLYSLSSVVTVPSSSLGTQTYICNVNHKPSNTKVDKRVEPKSC |
| | | DKTHTCPPCPAPEFEGGPSVFLFPPKPKDTLMISRTPEVTCVVVAVSHEDP |
| | | EVKFNWYVDGVEVHNAKTKPREEQYNSTYRVVSVLTVLHQDWLNGKEYK |
| | | CKVSNKALPAPIEKTISKAKGQPREPQVYTLPPSREEMTKNQVSLTCLVKGF |
| | | YPSDIAVEWESNGQPENNYKTTPPVLDSDGSFLLYSKLTVDKSRWQQGNV |
| | | FSCSVMHEALHNHYTQKSLSLSPG |
| 21 | Kappa-C | RTVAAPSVFIFPPSDEQLKSGTASVVCLLNNFYPREAKVQWKVDNALQSG |
| | | NSQESVTEQDSKDSTYSLSSTLTLSKADYEKHKVYACEVTHQGLSSPVTKSF |
| | | NRGEC |
| 22 | Lambda-C | GQPKAAPSVTLFPPSSEELQANKATLVCLISDFYPGAVTVAWKADSSPVKA |
| | | GVETTTPSKQSNNKYAASSYLSLTPEQWKSHRSYSCQVTHEGSTVEKTVAP |
| | | TECS |
| 23 | Human CD137 (UniProtKB- | MGNSCYNIVALLLVLNFERTRSLQDPCSNCPAGTFCDNNRNQICSPCPPN |
| | Q07011; incl. signal | SFSSAGGQRTCDICRQCKGVFRTRKECSSTSNAECDCTPGFHCLGAGCSM |
| | peptide sequence: aa 1- | CEQDCKQGQELTKKGCKDCCFGTFNDQKRGICRPWTNCSLDGKSVLVNG |
| | 23) | TKERDVVCGPSPADLSPGASSVTPPAPAREPGHSPQIISFFLALTSTALLFLLF |
| | | FLTLRFSVVKRGRKKLLYIFKQPFMRPVQTTQEEDGCSCRFPEEEEGGCEL |
| 24 | Human CD137 (UniProtKB- | LQDPCSNCPAGTFCDNNRNQICSPCPPNSFSSAGGQRTCDICRQCKGVFR |
| | Q07011; mature | TRKECSSTSNAECDCTPGFHCLGAGCSMCEQDCKQGQELTKKGCKDCCF |
| | sequence) | GTFNDQKRGICRPWTNCSLDGKSVLVNGTKERDVVCGPSPADLSPGASSV |
| | | TPPAPAREPGHSPQIISFFLALTSTALLFLLFFLTLRFSVVKRGRKKLLYIFKQP |
| | | FMRPVQTTQEEDGCSCRFPEEEEGGCEL |
| 25 | Human PD-L1 (UniProtKB- | MRIFAVFIFMTYWHLLNAFTVTVPKDLYVVEYGSNMTIECFPVEKQLDLAA |
| | Q9NZQ7; incl. signal | LIVYWEMEDKNIIQFVHGEEDLKVQHSSYRQRARLLKDQLSLGNAALQITD |
| | peptide sequence: aa 1- | VKLQDAGVYRCMISYGGADYKRITVKVNAPYNKINQRILVVDPVTSEHELT |
| | 18) | CQAEGYPKAEVIWTSSDHQVLSGKTTTTNSKREEKLFNVTSTLRINTTTNEI |
| | | FYCTFRRLDPEENHTAELVIPELPLAHPPNERTHLVILGAILLCLGVALTFIFRL |
| | | RKGRMMDVKKCGIQDTNSKKQSDTHLEET |
| 26 | Human PD-L1 (UniProtKB- | FTVTVPKDLYVVEYGSNMTIECKFPVEKQLDLAALIVYWEMEDKNIIQFVH |
| | Q9NZQ7; mature | GEEDLKVQHSSYRQRARLLKDQLSLGNAALQITDVKLQDAGVYRCMISYG |
| | sequence) | GADYKRITVKVNAPYNKINQRILVVDPVTSEHELTCQAEGYPKAEVIWTSS |
| | | DHQVLSGKTTTTNSKREEKLFNVTSTLRINTTTNEIFYCTFRRLDPEENHTAE |
| | | LVIPELPLAHPPNERTHLVILGAILLCLGVALTFIFRLRKGRMMDVKKCGIQD |
| | | TNSKKQSDTHLEET |
| 27 | Homo Sapiens EGFR | MRPSGTAGAALLALLAALCPASRALEEKKVCQGTSNKLTQLGTFEDHFLSL |
| | | QRMFNNCEVVLGNLEITYVQRNYDLSFLKTIQEVAGYVLIALNTVERIPLEN |
| | | LQIIRGNMYYENSYALAVLSNYDANKTGLKELPMRNLQEILHGAVRFSNNP |
| | | ALCNVESIQWRDIVSSDFLSNMSMDFQNHLGSCQKCDPSCPNGSCWGA |
| | | GEENCQKLTKIICAQQCSGRCRGKSPSDCCHNQCAAGCTGPRESDCLVCR |
| | | KFRDEATCKDTCPPLMLYNPTTYQMDVNPEGKYSFGATCVKKCPRNYVVT |
| | | DHGSCVRACGADSYEMEEDGVRKCKKCEGPCRKVCNGIGIGEFKDSLSIN |
| | | ATNIKHFKNCTSISGDLHILPVAFRGDSFTHTPPLDPQELDILKTVKEITGFLLI |
| | | QAWPENRTDLHAFENLEIIRGRTKQHGQFSLAVVSLNITSLGLRSLKEISDG |
| | | DVIISGNKNLCYANTINWKKLFGTSGQKTKIISNRGENSCKATGQVCHALC |
| | | SPEGCWGPEPRDCVSCRNVSRGRECVDKCNLLEGEPREFVENSECIQCHP |
| | | ECLPQAMNITCTGRGPDNCIQCAHYIDGPHCVKTCPAGVMGENNTLVW |
| | | KYADAGHVCHLCHPNCTYGCTGPGLEGCPTNGPKIPSIATGMVGALLLLLV |
| | | VALGIGLFMRRRHIVRKRTLRRLLQERELVEPLTPSGEAPNQALLRILKETEF |
| | | KKIKVLGSGAFGTVYKGLWIPEGEKVKIPVAIKELREATSPKANKEILDEAYV |
| | | MASVDNPHVCRLLGICLTSTVQLITQLMPFGCLLDYVREHKDNIGSQYLLN |
| | | WCVQIAKGMNYLEDRRLVHRDLAARNVLVKTPQHVKITDFGLAKLLGAEE |
| | | KEYHAEGGKVPIKWMALESILHRIYTQSDVWSYGVTVWELMTFGSKPYD |
| | | GIPASEISSILEKGERLPQPPICTIDVYMIMVKCWMIDADSRPKFRELIIEFSK |
| | | MARDPQRYLVIQGDERMHLPSPTDSNFYRALMDEEDMDDVVDADEYLI |
| | | PQQGFFSSPSTSRTPLLSSLSATSNNSTVACIDRNGLQSCPIKEDSFLQRYSS |
| | | DPTGALTEDSIDDTFLPVPEYINQSVPKRPAGSVQNPVYHNQPLNPAPSRD |
| | | PHYQDPHSTAVGNPEYLNTVQPTCVNSTFDSPAHWAQKGSHQISLDNPD |
| | | YQQDFFPKEAKPNGIFKGSTAENAEYLRVAPQSSEFIGA |
| 28 | VH_CD137-009 | QSLEESGGRLVTPGTPLTLTCTVSGFSLNDYWMSWVRQAPGKGLEWIGYI |
| | | DVGGSLYYASWAKGRFTISRTSTTVDLKMTSLTTEDTATYFCARGGLTYGF |
| | | DLWGPGTLVTVSS |
| 29 | VL_CD137-009 | DIVMTQTPASVSEPVGGTVTINCQASEDISSYLAWYQQKPGQRPKRLIYG |
| | | ASDLASGVPSRFSASGSGTEYALTISDLESADAATYYCHYYATISGLGVAFG |
| | | GGTEVVVK |
| 30 | m4-1BB-3H3 VH | EMQLVESGGGLVQPGRSMKLSCAGSGFTLSDYGVAWVRQAPKKGL |
| | | EWVAYISYAGGTTYYRESVKGRFTISRDNAKSTLYLQMDSLRSED |
| | | TATYYCTIDGYGGYSGSHWYFDFWGPGTMVTVSS |
| 31 | m4-1BB-3H3 VL | DIQMTQSPSLLSASVGDRVTLNCRTSQNVYKNLAWYQQKLGEAPK |
| | | LLIYNANSLQAGIPSRFSGSGSGTDFTLTISSLOPEDVATYFCQQ |
| | | YYSGNTFGAGTNLELK |
| 32 | MPDL3280A VH | EVOLVESGGGLVQPGGSLRLSCAASGFTFSDSWIHWYRQAPGKGLEWYA |
| | | WISPYGGSTYYADSVKGRFTISADTSKNTAYLQMNSLRAEDTAVYYCARR |
| | | HWPGGFDYWGQGTLVTVSS |
| 33 | MPDL3280A VL | DIQMTQSPSSLSASVGDRVTITCRASQDVSTAVAWYQQKPGKAPKLLIYSA |
| | | SFLYSGVPSRFSGSGSGTDFTLTISSLOPEDFATYYCQQYLYHPATFGQGTK |
| | | VEIK |
| 34 | AALT | AKTTAPSVYPLAPVCGDTTGSSVTLGCLVKGYFPEPVTLTWNSGSLSSGVH |
| | | TFPAVLQSDLYTLSSSVTVTSSTWPSQSITCNVAHPASSTKVDKKIEPRGPTI |
| | | KPCPPCKCPAPNAAGGPSVFIFPPKIKDVLMISLSPMVTCVVVDVSEDDPD |
| | | VQISWFVNNVEVLTAQTQTHREDYNSTLRVVSALPIQHQDWMSGKEFKC |
| | | KVNNKALPAPIERTISKPKGSVRAPQVYVLPPPEEEMTKKQVTLTCMVTDF |
| | | MPEDIYVEWTNNGKTELNYKNTEPVLDSDGSYLMYSKLTVEKKNWVERN |
| | | SYSCSVVHEGLHNHHTTKSFSRTPGK |
| 35 | AAKR | AKTTAPSVYPLAPVCGDTTGSSVTLGCLVKGYFPEPVTLTWNSGSLSSGVH |
| | | TFPAVLQSDLYTLSSSVTVTSSTWPSQSITCNVAHPASSTKVDKKIEPRGPTI |
| | | KPCPPCKCPAPNAAGGPSVFIFPPKIKDVLMISLSPMVTCVVVDVSEDDPD |
| | | VQISWFVNNVEVLTAQTQTHREDYNSTLRVVSALPIQHQDWMSGKEFKC |
| | | KVNNKALPAPIERTISKPKGSVRAPQVYVLPPPEEEMTKKQVTLTCMVKDF |
| | | MPEDIYVEWTNNGKTELNYKNTEPVLDSDGSYFMYSRLRVEKKNWVERN |
| | | SYSCSVVHEGLHNHHTTKSFSRTPGK |
| 36 | Constant region kappa LC | RADAAPTVSIFPPSSEQLTSGGASVVCFLNNFYPKDINVKWKIDGSERQNG |
| | | VLNSWTDQDSKDSTYSMSSTLTLTKDEYERHNSYTCEATHKTSTSPIVKSFN |
| | | RNEC |

The present invention is further illustrated by the following examples, which should not be construed as limiting the scope of the invention.

### EXAMPLES

### Example 1: Generation of CD137 Antibody

The antibodies CD137-005 and CD137-009 were generated as described in example 1 of WO2016/110584. In short, rabbits were immunized with a mixture of proteins containing a human CD137-Fc fusion protein. Single B cells from blood were sorted and screened for production of CD137 specific antibody by ELISA and flow cytometry. From screening-positive B cells, RNA was extracted and sequencing was performed. The variable regions of heavy and light chain were gene synthesized and cloned into a human IgG1 kappa expression vector or human IgG1 lambda expression vector including a human IgG1 heavy chain containing the following amino acid mutations: L234F, L235E, D265A and F405L (FEAL) or F405L (FEAL) wherein the amino acid position number is according to EU numbering (correspond to SEQ ID NO: 20). The variable region sequences of the chimeric CD137 antibody (CD137-009) are shown in the Sequence Listing SEQ ID NO: 28 and SEQ ID NO: 29 herein.

### Example 2: Humanization of the Rabbit (Chimeric) CD137 Antibody

Humanized antibody sequences from the rabbit anti-CD137-009 were generated at Antitope (Cambridge, UK). Humanized antibody sequences were generated using germline humanization (CDR-grafting) technology. Humanized V region genes were designed based on human germline sequences with closest homology to the VH and Vκ amino acid sequences of the rabbit antibody. A series of seven VH and three Vκ (VL) germline humanized V-region genes were designed. Structural models of the non-human parental antibody V regions were produced using Swiss PDB and analyzed in order to identify amino acids in the V region frameworks that may be important for the binding properties of the antibody. These amino acids were noted for incorporation into one or more variant CDR-grafted antibodies. The germline sequences used as the basis for the humanized designs are shown in Table 8.

TABLE 8
Closest matching human germline V segment and J segment sequences.

| | Heavy chain | | Light chain (K) | |
|---|---|---|---|---|
| | Human V region | Human J region | Human V region | Human J region |
| Antibody | germline segment | germline segment | germline segment | germline segment |
| Rabbit anti- | hIGHV3-49*04 | hIGHJ4 | hIGKV1-33*01 | IGKJ4 |
| CD137-009 | | | | |

Variant sequences with the lowest incidence of potential T cell epitopes were then selected using Antitope's proprietary in silico technologies, iTope™ and TCED™ (T Cell Epitope Database) (Perry, L. C. A, Jones, T. D. and Baker, M. P. New Approaches to Prediction of Immune Responses to Therapeutic Proteins during Preclinical Development (2008). Drugs in R&D 9 (6): 385-396; 20 Bryson, C. J., Jones, T. D. and Baker, M. P. Prediction of Immunogenicity of Therapeutic Proteins (2010). Biodrugs 24 (1):1-8). Finally, the nucleotide sequences of the designed variants have been codon-optimized.

The variable region sequences of the humanized CD137 antibody (CD137-009-HC7LC2) are shown in the Sequence Listing SEQ ID NO: 1 and SEQ ID NO: 5 herein.

### Example 3: Generation of PD-L1 Antibody

Immunization and hybridoma generation were performed at Aldevron GmbH (Freiburg, Germany). A cDNA encoding amino acid 19-238 of human PD-L1 was cloned into Aldevron proprietary expression plasmids. Antibody PD-L1-547 was generated by immunization of OmniRat animals (transgenic rats expressing a diversified repertoire of antibodies with fully human idiotypes; Ligand Pharmaceuticals Inc., San Diego, USA) using intradermal application of human PD-L1 cDNA-coated gold-particles using a hand-held device for particle-bombardment (“gene gun”). Serum samples were collected after a series of immunizations and tested in flow cytometry on HEK cells transiently transfected with the aforementioned expression plasmids to express human PD-L1. Antibody-producing cells were isolated and fused with mouse myeloma cells (Ag8) according to standard procedures. RNA from hybridomas producing PD-L1 specific antibody was extracted and sequencing was performed. The variable regions of heavy and light chain (SEQ ID NOs: 8 and 12) were gene synthesized and cloned into a human IgG1 lambda expression vector including a human IgG1 heavy chain containing the following amino acid mutations: L234F, L235E, D265A and K409R (FEAR) wherein the amino acid position number is according to EU numbering (correspond to SEQ ID NO: 19).

### Example 4: Generation of Bispecific Antibodies by 2-MEA-Induced Fab-Arm Exchange

Bispecific IgG1 antibodies were generated by Fab-arm-exchange under controlled reducing conditions. The basis for this method is the use of complementary CH3 domains, which promote the formation of heterodimers under specific assay conditions as described in WO2011/131746. The F405L and K409R (EU numbering) mutations were introduced into the relevant antibodies to create antibody pairs with complementary CH3 domains.

To generate bispecific antibodies, the two parental complementary antibodies, each antibody at a final concentration of 0.5 mg/mL, were incubated with 75 mM 2-mercaptoethylamine-HCl (2-MEA) in a total volume of 100 μL PBS at 31° C. for 5 hours. The reduction reaction was stopped by removing the reducing agent 2-MEA using spin columns (Microcon centrifugal filters, 30k, Millipore) according to the manufacturer's protocol.

Bispecific antibodies were generated by combining the following antibodies from Example 1 and 3
CD137-009-FEAL antibody combined with the PD-L1-547-FEAR antibody
PD-L1-547-FEAL antibody combined with the CD137-009-FEAR antibody
GEN1046 (PD-L1-547-FEAL antibody combined with CD137-009-HC7LC2-FEAR antibody (CD137 binding arm: SEQ ID NOs: 1, 5, 21, 19; PD-L1 binding arm: SEQ ID NOs: 8, 12, 22, 20),
b12-FEAL antibody combined with the PD-L1-547-FEAR antibody, with CD137-009-FEAR or with CD137-009-HC7LC2-FEAR antibody, using as the first arm the antibody b12 which is a gp120 specific antibody (Barbas, CF. J Mol Biol. 1993 Apr. 5; 230(3):812-23)
PD-L1-547-FEAL or CD137-009-FEAL with b12-FEAR antibody.

### Example 5: Pharmacodynamic Evaluation of GEN1046 in Peripheral Blood in Patients with Advanced Solid Tumors

To investigate the biological activity of GEN1046 at various dose levels in patients with advanced tumors, blood and serum samples were collected at baseline and at multiple timepoints on treatment. Based on the mechanism of action of GEN1046, it was anticipated that dose levels with biological activity will modulate circulating levels of interferon-γ and induce proliferation of peripheral CD8 T cells.

To determine serum levels of interferon-gamma (IFN-γ), serum samples were collected from patients at baseline and at multiple timepoints post administration of GEN1046 in cycle 1 and cycle 2 (days 1 [2 h and between 4-6 h post-administration], 2, 3, 8, and 15). Serum levels of IFN-γ were measured by a Meso Scale Discovery (MSD) multiplex immune-assay (cat. no. K15209G) following the manufacturer's instructions.

To measure peripheral modulation of immune cells subsets, immunophenotyping of peripheral blood was conducted in whole blood collected in EDTA tubes at baseline and at multiple timepoints post GEN1046 administration in cycle 1 and cycle 2 (days 2, 3, 8 and 15). 100 μL of whole blood was added to fluorochrome-conjugated monoclonal antibodies that bind specifically to cell surface antigens: CD45RA-FITC (clone LEU-18, BD Biosciences cat. no. 335039), CCR7-BV510 (clone 3D12, BD Biosciences, cat. no. 563449), CD8-PerCP-Cy5.5 (clone RPA-T8, BD Biosciences, cat. no. 560662). After incubation on ice, the stained samples were treated with FACS Lysing Solution (BD Biosciences, Catalog No 349202) to lyse erythrocytes. Excess antibody and cell debris were removed by washing with Stain Buffer (BD Biosciences, cat. no. 554656). Following lyse/wash, cells were fixed and permeabilized by incubation with Permeabilizing Solution 2 buffer (BD Biosciences, cat. no. 340973). Next, cells were washed and resuspended in Stain Buffer and incubated on ice with antibody to Ki67 (BV421 B56, BD Biosciences, cat. no. 562899) to detect proliferating cells. After incubation, excess antibody was removed by washing with Stain Buffer. Cells were resuspended in Stain Buffer and acquired on a BD FACSCanto™ II flow cytometer (Becton Dickinson) within 1 hour of staining.

Administration of GEN1046 to cancer patients resulted in modulation of circulating levels of IFN-γ and proliferating effector memory CD8 T cells (Table 1). Levels of IFN-γ increased more than 2-fold in the first treatment cycle across all dose levels tested. [symbol] Maximal increases were detected at the 50 mg and 80 mg dose levels, and most of the patients in the 80 mg cohort (75%) had fold-increase >2 (Table 1). GEN1046 also elicited proliferation of effector memory CD8⁺ T cells as measured by an increase in the frequency of Ki67⁺CD8⁺ CD45RA⁻CCR7⁻ T cells. Comparable to the changes observed with modulation of circulating levels of IFNγ, maximal and more consistent modulation of proliferating CD8+ effector memory T cells was observed in patients in the 80 mg cohort. Particularly in the 400 mg cohort the magnitude of the changes in both the circulating levels of IFN-γ and proliferating effector memory CD8 T cells were lower compared to the 25-200 mg cohorts. These results showed that GEN1046 elicited an immune response characterized by modulation of immune effector cells and soluble factors critical for the generation of antitumor immune responses, with responses of greater magnitude at the 80 mg dose level.

TABLE 9
GEN1046 Modulation of Peripheral Pharmacodynamic Endpoints in cancer patients:
Peak Fold-change from Baseline during Cycle 1 by Dose Level^{a}

| | GEN1046 | GEN1046 | GEN1046 | GEN1046 | GEN1046 |
|---|---|---|---|---|---|
| | 25 mg | 50 mg | 80 mg | 200 mg | 400 mg |
| Interferon-γ^{b} | | | | | |
| n | 4 | 4 | 8 | 8 | 6 |
| Min | 1.17 | 1.06 | 1.45 | 1.47 | 1.18 |
| Q1 | 2.05 | 1.89 | 2.82 | 2.35 | 1.32 |
| Median | 3.90 | 4.63 | 4.49 | 3.48 | 2.56 |
| Q3 | 9.99 | 6.90 | 5.94 | 4.89 | 3.37 |
| Max | 15.11 | 7.27 | 12.17 | 5.20 | 102.08 |
| Proliferating Effector | | | | | |
| Memory CD8 T cells^{c} | | | | | |
| n | 3 | 2 | 8 | 8 | 7 |
| Min | 2.00 | 2.00 | 1.00 | 0.67 | 1.00 |
| Q1 | 2.00 | 2.00 | 2.00 | 1.40 | 1.06 |
| Median | 2.00 | 2.50 | 3.42 | 2.83 | 1.50 |
| Q3 | 3.50 | 3.00 | 9.75 | 5.25 | 2.00 |
| Max | 5.00 | 3.00 | 31.40 | 6.67 | 7.00 |

Preliminary data as of 27 Jan. 2020.
n: number of patients per dose cohort; Min: lowest measured value; Q1: 25th percentile; Q3: 75th percentile; Max: maximum measured value.
^{a}Pharmacodynamic assessments, including changes in circulating levels of interferon-gamma and effector memory T cells, were conducted using blood samples from patients with advanced solid tumors enrolled in the dose escalation phase of an open-label, multi-center safety trial of GEN1046 (NCT03917381).
^{b}Circulating levels of interferon-gamma were measured in serum samples at baseline, and at multiple timepoints post administration of GEN1046 in cycle 1 and cycle 2 (days 1 [2 h and between 4-6 h post-administration], 2, 3, 8, and 15). Interferon-gamma levels in serum samples were determined by Meso Scale Discovery (MSD) multiplex immune assay.
^{c}Immunophenotyping of peripheral blood was conducted in whole blood collected at baseline and at multiple timepoints post administration of GEN1046 in cycle 1 and cycle 2 (days 2, 3, 8 and 15). The frequency of proliferating (Ki67⁺) effector memory CD8 T cells (CD8⁺CD45RA⁻CCR7⁻ T cells) were assessed in whole blood samples by flow cytometry.

### Example 6: Preliminary Data from Dose Escalation

Trial design:

Clinical trial on GCT1046-01 (ClinicalTrials.gov Identifier: NCT03917381) was designed as a two-part trial, including an ongoing dose escalation part and a planned expansion part.

The trial was designed as an open-label, multi-center, Phase I/IIa safety trial of GEN1046 (DuoBody*-PD-L1×4-1BB). The trial consists of 2 parts; a First-in-Human (FIH) dose escalation (Phase I) and an expansion (Phase 11a). FIG. 2 shows a schematic representation of the clinical trial design.

Dose Escalation

The dose escalation was designed to evaluate GEN1046 in subjects with solid malignant tumors to determine the maximum tolerated dose (MTD) or maximum administered dose (MAD) and/or the recommended phase 2 dose (RP2D).

For dose escalation, subject was required to be a man or woman 18 years of age and was required to have measurable disease according to RECIST 1.1.

Subjects was required to have a histologically or cytologically confirmed non-CNS solid tumor that was metastatic or unresectable and for whom there was no available standard therapy likely to confer clinical benefit, or subjects who are not candidates for such available therapy, and for whom, in the opinion of the investigator, experimental therapy with GEN1046 could be beneficial.

In the dose escalation, subjects received one infusion of GEN1046 every third week (1Q3W) until protocol defined treatment discontinuation criteria are met; e.g. Radiographic disease progression or clinical progression. GEN1046 was administered using i.v. infusion over a minimum of 60 minutes on Day 1 of each 3-week treatment cycle (21 days). The concept of the design of the trial is shown in FIG. 2.

The 1Q3W dose escalation was designed to potentially (dependent on data collected during the trial) evaluate GEN1046 at 7 main dose levels: 25, 80, 200, 400, 800, 1200 and 1600 mg fixed, and 6 optional intermediate dose levels 50, 140, 300, 600, 1000 and 1400 mg fixed.

The recommended phase 2 dose (RP2D) was based on a review of the available safety and dosing information and could be lower than the maximum tolerated dose (MTD).

Expansion

The aim of the expansion is to provide further data on the safety, tolerability, MoA, PK and anti-tumor activity of the selected dose/schedule.

Expansion was designed to initiate recruitment in up to 6 tumor types (7 parallel cohorts), i.e., in NSCLC, EC, UC, TNBC, SCCHN, and cervical cancer. Further expansion cohorts in additional tumor types may be opened based on preliminary efficacy signals generated in the dose escalation. The sponsor will determine the priority of opening the disease-specific expansion cohorts based on the data obtained in the dose escalation.

NSCLC Expansion Cohorts

The NSCLC expansion cohorts should include subjects with squamous histology as well as subjects with non-squamous histology.

Since response rates and other disease related outcomes may differ in a PD-1/PD-L1 naïve population versus a PD-1/L1 pre-treated population, NSCLC patients were separated into different cohorts to ensure sufficient evidence of preliminary efficacy. Cohort 2 aims to explore preliminary efficacy in PD-1/L1 naive patients with NSCLC where SOC with PD-1/L1 inhibitors is restricted or unavailable. If preliminary clinical evidence suggests a substantial improvement over available therapies in a population with high unmet medical need (e.g., PD-L1 low or negative) as determined by the DMC's review of the totality of the data, the Sponsor may request to open Cohort 2 in areas where access to PD-1/L1 inhibitors is not restricted.

UC Expansion Cohort

The UC cohort was designed to include both subjects who are eligible to receive platinum-based chemotherapy and subjects who are not eligible to receive platinum-based chemotherapy.

SCCHN and TNBC Expansion Cohorts

The SCCHN and TNBC cohorts may include both subjects who have received prior treatment with a PD-1/PD-L1 inhibitor and subjects who have not received treatment with a prior PD-1/L1 inhibitor.

Inclusion Criteria

Subjects are eligible to be included in the trial only if all of the following criteria apply:

Subject must be a man or woman 18 years of age Subject and must have measurable disease according to RECIST 1.1.

Subjects must have histologically or cytological confirmed diagnosis of relapsed or refractory, advanced and/or metastatic NSCLC, EC, UC, TNBC, SCCHN, or cervical cancer who are no longer candidates for or refuse standard therapy (if subjects had access and were eligible for the respective treatments), and who have failed anticancer therapy as follows:

Expansion Cohort 1 (NSCLC): PD-1/L1 Pre-Treated

NSCLC subjects who have received up to 4 prior systemic treatment regimens (adjuvant and maintenance treatment is considered being part of one treatment line) for advanced/metastatic disease with radiographic disease progression on or after last prior treatment.

NSCLC subjects of any histology may be enrolled. Subjects with a histological or cytological diagnosis of non-squamous NSCLC must not have an epidermal growth factor (EGFR)-sensitizing mutation and/or anaplastic lymphoma (ALK) translocation/ROS1 rearrangement. EGFR sensitizing mutations are those mutations that are amenable to treatment with an approved tyrosine kinase inhibitor (TKI). Documentation of EGFR and ALK status should be available per local assessment. If documentation of EGFR and ALK status is unavailable, sponsor medical monitor approval is required prior to enrollment.

Subjects should have received platinum-based therapy (or alternative chemotherapy due to platinum ineligibility, e.g., a gemcitabine-containing regimen).

Subjects must have received prior treatment with a PD-1/L1 inhibitor alone or in combination and must have radiographic disease progression on treatment. Sponsor approval is required for subjects with a BOR of SD or PD on a CPI containing regimen with a treatment duration of up to 16 weeks.

Expansion Cohort 2 (NSCLC)—PD-1/L1 Naive

NSCLC subjects who have received up to 4 prior systemic treatment regimens (maintenance treatment is considered being part of one treatment line) for metastatic disease with radiographic disease progression on or after last prior treatment.

NSCLC subjects of any histology may be enrolled. Subjects with a histological or cytological diagnosis of non-squamous NSCLC must not have an epidermal growth factor (EGFR)-sensitizing mutation and/or anaplastic lymphoma kinase (ALK) translocation/ROS1 rearrangement. EGFR sensitizing mutations are those mutations that are amendable to treatment with an approved tyrosine kinase inhibitor (TKI). Documentation of EGFR and ALK status should be available per local assessment. If documentation of EGFR and ALK status is unavailable, sponsor medical monitor approval is required prior to enrollment.

Subjects should have received platinum-based therapy (or alternative chemotherapy due to platinum ineligibility, e.g., a gemcitabine-containing regimen).

Subjects must not have received prior treatment with a PD-1/L1 inhibitor.

Expansion Cohort 3 (UC):

UC (of the bladder, ureter, urethra, or renal pelvis) subjects who have received up to 4 prior systemic treatment regimens (adjuvant and maintenance treatment is considered being part of one treatment line) for locally advanced/metastatic disease with radiographic disease progression on or after last prior treatment.

Subjects must have received prior treatment with a PD-1/L1 inhibitor alone or in combination and must have radiographic disease progression on treatment. Sponsor approval is required for subjects with a BOR of SD or PD on a CPI containing regimen with a treatment duration of up to 16 weeks.

Local results from the most recent PD-L1 test should be provided prior to enrollment (if available).
Cohort 3a: For subjects who are eligible to receive platinum-based therapy: Subjects must have received platinum-based chemotherapy.
Cohort 3b: For subjects ineligible to receive platinum-based therapy: Subjects must not be eligible for any platinum-based or any cisplatin-containing chemotherapy.

Expansion Cohort 4 (EC):

EC subjects who have received up to 4 prior systemic treatment regimens (maintenance treatment is considered being part of one treatment line) for advanced/metastatic disease with radiographic disease progression on or after last prior treatment.

Subjects must have epithelial endometrial histology including: endometrioid, serous, squamous, clear-cell carcinoma, or carcinosarcoma. Sarcomas and mesenchymal EC are excluded.

Subjects must not have received prior treatment with a PD-1/L1 inhibitor (established local label/access need to be respected).

Expansion Cohort 5 (TNBC):

TNBC defined as HER2-negative [HER2 is negative by FISH] assay (non-amplified ratio of HER2 to CEP17 <2.0 single probe average HER2 gene copy number<4 signals/cell) or alternatively HER2 protein expression by IHC result is 1+ negative or IHC 0− negative and ER and PgR negative status (defined as <1% of cells expressing hormonal receptors via IHC analysis) as per local assessment. Subjects who have received up to 4 prior systemic treatment regimens including but not limited to anthracycline-, taxane-, antimetabolite- or microtubule inhibitor-containing regimens (maintenance treatment is considered being part of one treatment line) for locally advanced/metastatic disease with radiographic disease progression on or after last prior treatment.

Subjects with a prior history of a breast cancer with a different phenotype must have confirmation of TNBC from a biopsy obtained after the subject's last prior systemic therapy.
Cohort 5a—Subjects who have received prior treatment with a PD-1/L1 inhibitor: Subjects must have received prior treatment with a PD-1/L1 inhibitor alone or in combination and must have radiographic disease progression on treatment.
Cohort 5b—Subjects who have not received prior treatment with a PD-1/L1 inhibitor: Subjects must not have received prior treatment with a PD-1/L1 inhibitor.

Expansion Cohort 6 (SCCHN):

Recurrent or metastatic SCCHN (oral cavity, pharynx, larynx) subjects who have received up to 4 prior systemic treatment regimens for recurrent/metastatic disease with radiographic PD on or after last prior treatment (maintenance treatment is considered being part of one treatment line).

Subjects must have disease progression on or after prior therapy with platinum-based chemotherapy (alternative combination chemotherapy is acceptable if the subject's platinum ineligibility status is documented).
Cohort 6a—Subjects who have received prior treatment with a PD-1/L1 inhibitor: Subjects must have received prior treatment with a PD-1/L1 inhibitor alone or in combination and must have radiographic disease progression on treatment. Sponsor approval is required for subjects with a BOR of SD or PD on a CPI containing regimen with a treatment duration of up to 16 weeks.
Cohort 6b—Subjects who have not received prior treatment with a PD-1/L1 inhibitor: Subjects must not have received prior treatment with a PD-1/L1 inhibitor.

Expansion Cohort 7 (Cervical Cancer):

Cervical cancer subjects who have received up to 4 prior systemic treatment regimens including chemotherapy in combination with bevacizumab (according to the applicable labeling) unless the subject is ineligible for bevacizumab according to local standards (chemotherapy administered in the adjuvant or neoadjuvant setting, or in combination with radiation therapy should not be counted as a prior line of therapy) for recurrent/metastatic disease with radiographic disease progression on or after last prior treatment.

Subjects must have cervical cancer of squamous cell, adenocarcinoma, or adenosquamous histology.

Subjects must not have received prior treatment with a PD-1/L1 inhibitor (established local label/access need to be respected).

Results

Dose Escalation

The following preliminary results were obtained during dose escalation. Table 10 shows Best Overall Response (RECIST v1.1) by Dose Level upon enrolment and dosing of a total of 30 patients (Data Extraction Date: 3 Feb. 2020).

Tables 11 and 12 show Objective Response Rate and Confirmed Objective Response Rate, respectively (RECIST v1.1) by Dose Level upon enrolment and dosing of a total of 61 patients (Data cut-off: Oct. 12, 2020).

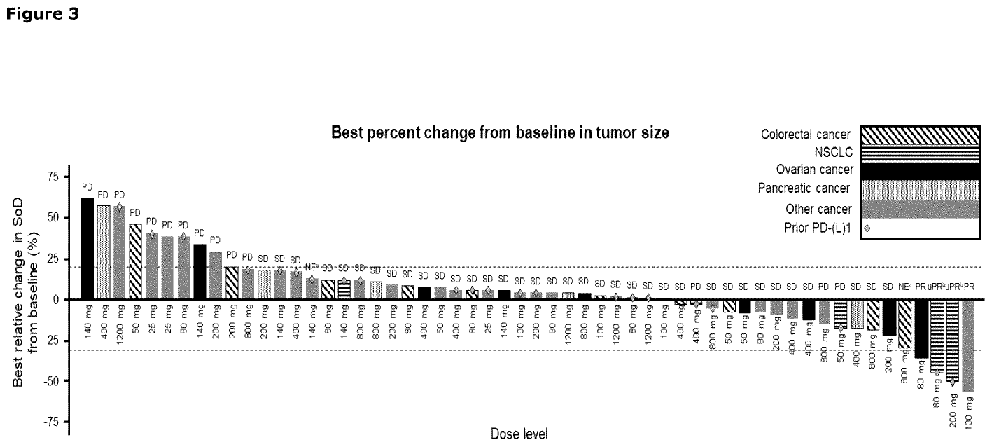

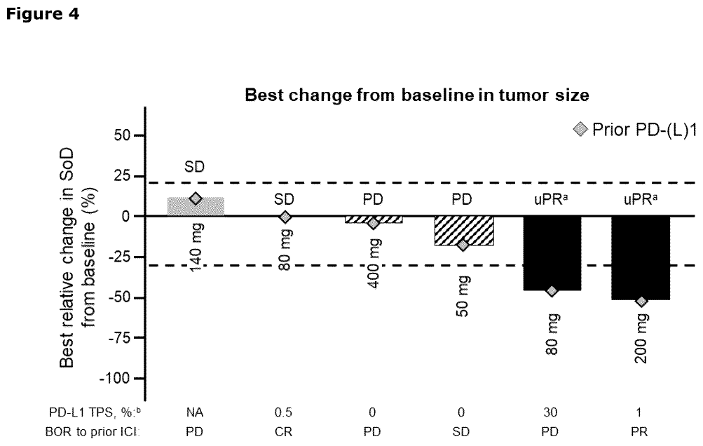

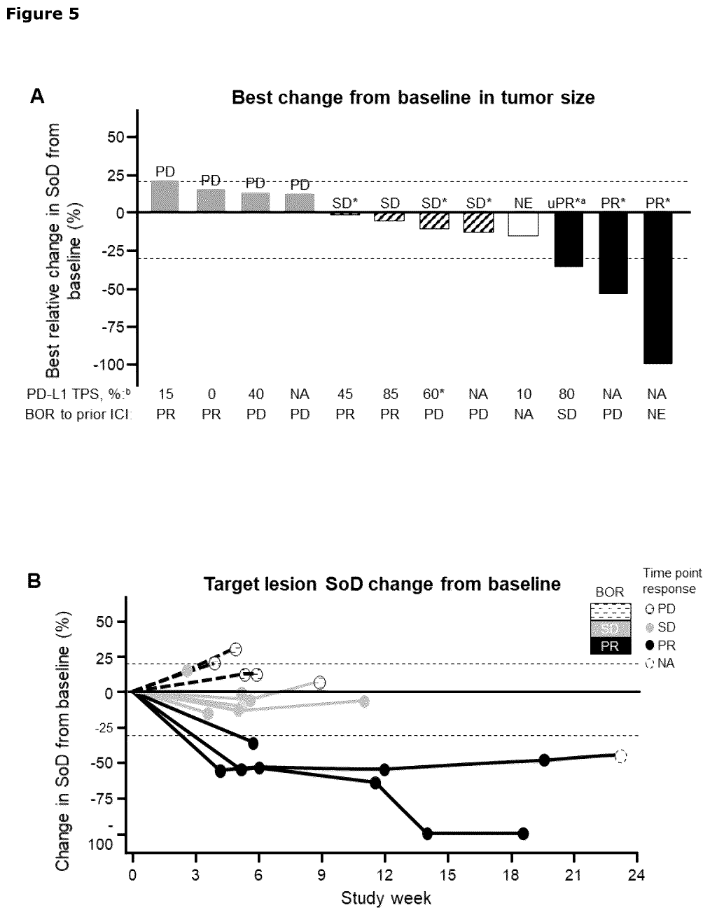

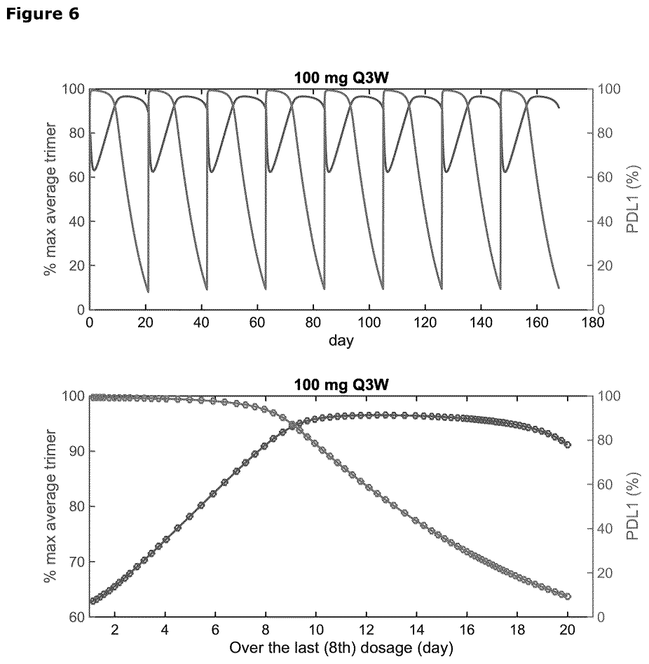

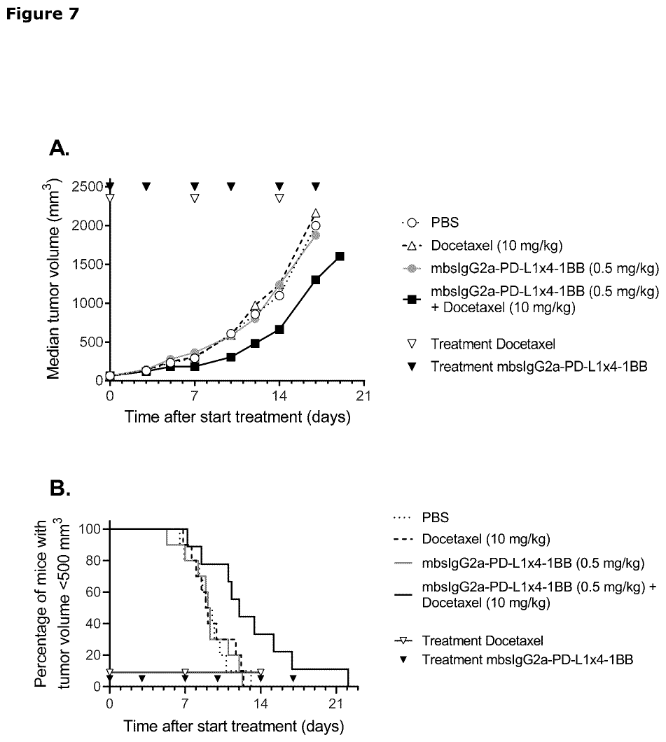

Best percent change from baseline in tumor size in all patients is shown in FIG. 3. Disease control occurred in 40/61 (65.6%) patients in the dose escalation phase. Partial response (PR) was achieved in four patients with triple-negative breast cancer, ovarian cancer, or non-small cell lung cancer (NSCLC); 36 patients maintained stable disease.

Clinical activity observed in patients with NSCLC (best change from baseline in tumor size) is show in in FIG. 4 (Data cut-off: Oct. 12, 2020). Of six patients with NSCLC, all of whom had received prior checkpoint immunotherapy, two achieved unconfirmed PR, two maintained stable disease, and two experienced progressive disease.

TABLE 10
Best Overall Response (RECIST v1.1) by Dose Level.

| | 25 mg | | 50 mg | | 80 mg | | 140 mg | | 200 mg | | 400 mg | | Total | |
|---|---|---|---|---|---|---|---|---|---|---|---|---|---|---|
| | (n = 2) | | (n = 5) | | (n = 8) | | (n = 1) | | (n = 8) | | (n = 6) | | (n = 30) | |
| Complete | 0 | (0) | 0 | (0) | 0 | (0) | 0 | (0) | 0 | (0) | 0 | (0) | 0 | (0) |
| Response | | | | | | | | | | | | | | |
| Partial | 0 | (0) | 0 | (0) | 2¹ | (25) | 0 | (0) | 1¹ | (12.5) | 0 | (0) | 3 | (10) |
| Response | | | | | | | | | | | | | | |
| Stable | 0 | (0) | 3 | (60) | 5 | (62.5) | 0 | (0) | 5 | (62.5) | 6 | (100) | 19 | (63.3) |
| Disease | | | | | | | | | | | | | | |
| Progressive | 2 | (100) | 2 | (40) | 1 | (12.5) | 1 | (100) | 2 | (25) | 0 | (0) | 8 | (26.6) |
| Disease | | | | | | | | | | | | | | |

¹uPR

TABLE 11
Objective Response Rate - dose escalation

| | Total | | 25 mg | 50 mg | 80 mg | 100 mg | 140 mg | 200 mg | 400 mg | 800 mg | 1200 mg |
|---|---|---|---|---|---|---|---|---|---|---|---|
| N | 61 | | 4 | 5 | 9 | 6 | 6 | 9 | 9 | 9 | 4 |
| Best Overall | | | | | | | | | | | |
| Response | | | | | | | | | | | |
| CR (Complete | 0 | | 0 | 0 | 0 | 0 | 0 | 0 | 0 | 0 | 0 |
| Response) | | | | | | | | | | | |
| PR (Partial | 4 | (6.6%) | 0 | 0 | 2 (22.2%) | 1 (16.7%) | 0 | 1 (11.1%) | 0 | 0 | 0 |
| Response) | | | | | | | | | | | |
| SD (Stable | 36 | (59.0%) | 1 (25.0%) | 3 (60.0%) | 6 (66.7%) | 3 (50.0%) | 3 (50.0%) | 5 (55.6%) | 7 (77.8%) | 5 (55.6%) | 3 (75.0%) |
| Disease) | | | | | | | | | | | |
| PD (Progressive | 14 | (23.0%) | 2 (50.0%) | 2 (40.0%) | 1 (11.1%) | 0 | 2 (33.3%) | 2 (22.2%) | 2 (22.2%) | 2 (22.2%) | 1 (25.0%) |
| Disease) | | | | | | | | | | | |
| NE (Not | 7 | (11.5%) | 1 (25.0%) | 0 | 0 | 2 (33.3%) | 1 (16.7%) | 1 (11.1%) | 0 | 2 (22.2%) | 0 |
| Evaluable) | | | | | | | | | | | |
| Objective | 4 | (6.6%) | 0 | 0 | 2 (22.2%) | 1 (16.7%) | 0 | 1 (11.1%) | 0 | 0 | 0 |
| Response | | | | | | | | | | | |
| (CR + PR) Rate | | | | | | | | | | | |
| Disease Control | 40 | (65.6%) | 1 (25.0%) | 3 (60.0%) | 8 (88.9%) | 4 (66.7%) | 3 (50.0%) | 6 (66.7%) | 7 (77.8%) | 5 (55.6%) | 3 (75.0%) |
| (PR + PR + | | | | | | | | | | | |
| SD) Rate | | | | | | | | | | | |

TABLE 12
Confirmed Response Rate - dose escalation

| | Total | | 25 mg | 50 mg | 80 mg | 100 mg | 140 mg | 200 mg | 400 mg | 800 mg | 1200 mg |
|---|---|---|---|---|---|---|---|---|---|---|---|
| N | 61 | | 4 | 5 | 9 | 6 | 6 | 9 | 9 | 9 | 4 |
| Confirmed Best | | | | | | | | | | | |
| Overall Response | | | | | | | | | | | |
| CR (Complete | 0 | | 0 | 0 | 0 | 0 | 0 | 0 | 0 | 0 | 0 |
| Response) | | | | | | | | | | | |
| PR (Partial | 2 | (3.3%) | 0 | 0 | 1 (11.1%) | 1 (16.7%) | 0 | 0 | 0 | 0 | 0 |
| Response) | | | | | | | | | | | |
| SD (Stable | 38 | (62.3%) | 1 (25.0%) | 3 (60.0%) | 7 (77.8%) | 3 (50.0%) | 3 (50.0%) | 6 (66.7%) | 7 (77.8%) | 5 (55.6%) | 3 (75.0%) |
| Disease) | | | | | | | | | | | |
| PD (Progressive | 14 | (23.0%) | 2 (50.0%) | 2 (40.0%) | 1 (11.1%) | 0 | 2 (33.3%) | 2 (22.2%) | 2 (22.2%) | 2 (22.2%) | 1 (25.0%) |
| Disease) | | | | | | | | | | | |
| NE (Not | 7 | (11.5%) | 1 (25.0%) | 0 | 0 | 2 (33.3%) | 1 (16.7%) | 1 (11.1%) | 0 | 2 (22.2%) | 0 |
| Evaluable) | | | | | | | | | | | |
| Confirmed | 2 | (3.3%) | 0 | 0 | 1 (11.1%) | 1 (16.7%) | 0 | 0 | 0 | 0 | 0 |
| Objective | | | | | | | | | | | |
| Response (CR + | | | | | | | | | | | |
| PR) Rate | | | | | | | | | | | |
| Confirmed Disease | 40 | (65.6%) | 1 (25.0%) | 3 (60.0%) | 8 (88.9%) | 4 (66.7%) | 3 (50.0%) | 6 (66.7%) | 7 (77.8%) | 5 (55.6%) | 3 (75.0%) |
| Control (PR + | | | | | | | | | | | |
| PR + SD) Rate | | | | | | | | | | | |

Expansion:

Expansion cohort 1: As of Oct. 12, 2020, 24 patients were enrolled in expansion cohort 1, which includes patients with NSCLC (PD-1/L1 pre-treated). 12 patients could be assessed post-baseline, with confirmed progression on or after checkpoint inhibitor therapy (FIG. 5).

Conclusions:

GEN1046 is a first-in-class, next-generation, PD-L1×4-1BB bispecific antibody with an acceptable safety profile and encouraging early clinical activity, unlike the existing 4-1BB agonists.

In the dose escalation phase of this phase I/IIa study, GEN1046 demonstrated a manageable safety profile and preliminary clinical activity in a heavily pretreated population with advanced solid tumors.

Most adverse events were mild to moderate; treatment-related Grade 3 transaminase elevations resolved with corticosteroids. No treatment-related bilirubin increases or Grade 4 transaminase elevations were observed. Six patients had dose limiting toxicities (DLTs); Maximum tolerated dose (MTD) was not reached.

Clinical benefit across different dose levels was observed in patients, including those resistant to prior immunotherapy and those with tumors typically less sensitive to immune checkpoint inhibitors (ICIs).

Disease control was achieved in 65.6% of patients, including partial responses in triple negative breast cancer (1), ovarian cancer (1), and ICI pre-treated NSCLC (2).

Modulation of pharmacodynamic endpoints was observed across a broad range of dose levels demonstrating biological activity.

Encouraging preliminary responses were observed in the expansion cohort currently enrolling patients with NSCLC, who had received prior checkpoint immunotherapy exposure.

### Example 7: Pharmacokinetic/Pharmacodynamic Model

An integrated semi-mechanistic PK/PD (Pharmacokinetic/Pharmacodynamic) model was developed that assumes distribution of GEN1046 into central and peripheral PK compartments, as well as partitioning into tumor and lymph compartments. The model leverages PK and pharmacodynamic data as well as physiological parameters from literature for parameterizations of expressions of PD-L1 and 4-1BB, and T-cell trafficking into these cells. Model compartments consist of well-mixed 2- and 3-dimensional spaces and free drug transfer between all compartments. In addition, the model incorporates dynamic binding of GEN1046 to PD-L1 and 4-1BB to predict trimer (crosslinking to PD-L1 and 4-1BB) formation and receptor occupancy (RO) for PD-L1 and 4-1BB in tumor. Simulations showed that trimer formation is optimal at a dose of 80 mg, and model predicted RO in tumor for PD-L1 and 4-1BB was deemed sufficient at doses between 80 to 140 mg. Increasing doses≥200 mg resulted in reduced trimer formation. In addition, based on available clinical pharmacodynamic data, higher magnitude and consistent modulation of peripheral pharmacodynamic endpoints (IFNγ and proliferating Ki67+ effector memory CD8⁺ T cells) were seen at dose levels≥200 mg. In light of PK/pharmacodynamic modeling predictions and available clinical data, the optimal dose of GEN1046 was predicted to be in the range of 80 to 140 mg. At 100 mg dose 1Q3W, maximal trimer formation and average RO for PD-L1 (%) is maintained at reasonable levels during the entire dosing interval.

Model Predicted Maximal Trimer Formation and Receptor Occupancy for PDL1 at 100 mg 1Q3W is shown in FIG. 6.

### Example 8: GEN1046 in Combination with Docetaxel in a MC38 Syngeneic Mouse Tumor Model

Methods

MC38 mouse colon cancer cells were cultured in Dulbecco's Modified Eagle Medium supplemented with 10% heat-inactivated fetal bovine serum at 37° C., 5% CO₂. MC38 cells were harvested from a cell culture growing in log-phase and quantified.

MC38 cells (1×10⁶ tumor cells in 100 μL PBS) were injected subcutaneously in the right lower flank of female C57BL/6 mice (obtained from Vital River Laboratories Research Models and Services; age 6-8 weeks at start of experiment).

Tumor growth was evaluated three times per week using a caliper. Tumor volumes (mm³) were calculated from caliper measurements as ([length]×[width]²)/2, where the length is the longest tumor dimension, and the width is the longest tumor dimension perpendicular to the length.

Mice were randomized into groups (n=10/group) with an average tumor volume of 64 mm³ prior to treatment. On treatment days, the mice were injected intraperitoneally with mbsIgG2a-PD-L1×4-1BB (0.5 mg/kg; injection volume of 10 μL/g body weight; two doses weekly for three weeks [2QW×3]), docetaxel (10 mg/kg; injection volume of 10 μL/g body weight; QW×3), a combination of mbsIgG2a-PD-L1×4-1BB (0.5 mg/kg; 2QW×3) with docetaxel (10 mg/kg; in two separate injections [mbsIgG2a-PD-L1×4-1BB followed by docetaxel after 30 min] with an injection volume of 10 μL/g body weight; QW×3), or PBS with an injection volume of 10 μL/g body weight (Table 13).

The mice were monitored daily for clinical signs of illness. Body weight measurements were performed three times a week after randomization. The experiment ended for the individual mice when the tumor volume exceeded 1500 mm³ or when the animals reached humane endpoints (e.g. when mice showed body weight loss >20%, when tumors showed ulceration [>75%], when serious clinical signs were observed and/or when the tumor growth blocked the physical activity of the mouse).

TABLE 13
Treatment groups and dosing regimen

| Treatment | N per | | | Dosing | Dosing | Seq IDs/ |
|---|---|---|---|---|---|---|
| group | group | Treatment | Dose ^{a} | route | regimen | Supplier, cat. no. |
| 1 | 10 | PBS | N/A | IP | 2QW × 3 ^{a} | N/A |
| 2 | 10 | Docetaxel | 10 mg/kg | IP | QW × 3 ^{a} | Hengrui Medicine |
| 3 | 10 | mbslgG2a-PD- | 0.5 mg/kg | IP | 2QW × 3 ^{a} | SEQ ID NOs: 30-36 |
| | | L1x4-1BB | | | | |
| 4 | 10 | mbslgG2a-PD- | 0.5 mg/kg + | IP | 2QW × 3 + | SEQ ID NOs: 30-36 |
| | | L1x4-1BB + | 10 mg/kg | | QW × 3 ^{a} | Hengrui Medicine |
| | | Docetaxel | | | | |

^{a} 2QW × 3: two doses weekly for three weeks

Results

Rapid tumor outgrowth was observed in MC38-bearing mice treated with PBS (FIG. 7A). In mice treated with docetaxel (10 mg/kg; QW×3) or mbsIgG2a-PD-L1×4-1BB (0.5 mg/kg; 2QW×3) tumor outgrowth was comparable to the PBS-treated group (FIG. 7A). However, in mice treated with mbsIgG2a-PD-L1×4-1BB (0.5 mg/kg; 2QW×3) combined with docetaxel (10 mg/kg; QW×3) tumor outgrowth was delayed (FIG. 7A). Kaplan-Meier analysis showed that treatment with the combination of mbsIgG2a-PD-L1×4-1BB and docetaxel induced a significant increase in progression-free survival, defined as the percentage of mice with tumor volume smaller than 500 mm³, when compared to the PBS-treated group (p<0.01) and compared to mbsIgG2a-PD-L1×4-1BB or Docetaxel alone (p<0.05; Mantel-Cox; FIG. 7B, Table 14). Hence, these findings are consistent with potentiated anti-tumor activity by the combination of Docetaxel and mbsIgG2a-PD-L1×4-1BB. Furthermore, the combination treatment was well-tolerated as no significant body weight loss was observed.

TABLE 14
Mantel-Cox analysis of the progression-free survival induced
by mbsIgG2a-PD-L1x4-1BB, docetaxel (either alone or in
combination) in the MC38 model in C57BL/6 mice.

| | | | Progression-free |
|---|---|---|---|
| | | | survival¹ |
| Treatment groups compared | | | Mantel-Cox P value |
| PBS | vs | docetaxel | 0.994 |
| PBS | vs | mbsIgG2a-PD-L1x4-1BB | 0.967 |
| PBS | vs | mbsIgG2a-PD-L1x4-1BB + | 0.009 |
| | | docetaxel | |
| Docetaxel | vs | mbsIgG2a-PD-L1x4-1BB | 0.785 |
| Docetaxel | vs | mbsIgG2a-PD-L1x4-1BB + | 0.033 |
| | | docetaxel | |
| mbsIgG2a-PD- | vs | mbsIgG2a-PD-L1x4-1BB + | 0.022 |
| L1x4-1BB | | docetaxel | |

¹Tumor volume <500mm³ was used as the cut-off for progression-free survival. Mantel-Cox analysis was performed after progression of all animals to a tumor volume above 500 mm³.

These results provide rationale for evaluating the combination of GEN1046 with docetaxel in cancer patients to increase anti-tumor efficacy and enhance survival.

### Example 9: Combination with Docetaxel

In the dose escalation portion of the trial, no MTD was determined, and doses up to 1200 mg 1Q3W were administered safely. The determination of dose was based on the totality of the available clinical efficacy and safety data, and the magnitude and consistency of immune modulation of PD endpoints and PK/PD model predictions. Initial safety data indicated that the occurrence of the most frequent adverse events such as ALT/AST elevations are not dose dependent and would therefore not require a lower starting dose for GEN1046 for safety reasons. Preliminary efficacy was observed at doses of 80 to 200 mg Q3W (Example 6), and clinical pharmacodynamics endpoints indicated consistent modulation of proliferating (Ki67⁺) effector memory CD8⁺ T cells and circulating levels of IFNγ at dose levels of ≥200 mg Q3W. These results were in line with predicted optimal trimer formation and average receptor occupation for PD-L1 blockade in tumor, which was observed at doses in the range of 80 to 140 mg Q3W using mechanistic modeling. The most frequent adverse events reported in patient receiving GEN1046 were transaminase elevations, followed by hyperthyroidism, fatigue, nausea, asthenia and neutrophil count decrease. Although mild or moderate increase of transaminases has been reported in NSCLC cancer patients receiving 75 mg/m² of docetaxel, these are not frequently seen side effects. Other common side effects of docetaxel, which overlap with adverse reactions seen with GEN1046, include fatigue, nausea, asthenia, and neutrophil count decreased. However, these events are not seen as frequently with GEN1046 as they are with docetaxel, and the vast majority of the events are mild or moderate.

Initial evaluation of GEN1046 in combination with docetaxel wa conducted in subjects with metastatic NSCLC who had progressed on platinum-based chemotherapy and PD-L1 inhibitor therapy. GEN1046 was administered in combination with Docetaxel (Taxotere®) using IV infusion on Day 1 of each 3-week treatment cycle until disease progression or until one of the predefined discontinuation of treatment criterion had been met. The docetaxel infusion began at least 30 minutes after the administration of GEN1046. Based on the limited potential for added toxicity with the combination and the lack of a clear dose dependency of GEN1046 with adverse events and efficacy, the GEN1046 dose was fixed at 100 mg. Docetaxel was administered at a dose of 55 mg/m² or 75 mg/m² depending on the number of prior Dose Limiting Toxicities observed. The maximum tested dose of docetaxel was be 75 mg/m². The GEN1046 dose of 100 mg 1Q3W used for the combination treatment was the Recommended Phase 2 Dose (RP2D), determined in the dose escalation part of the trial.

The following inclusion criteria applied to the combination arm (NSCLC): GEN1046 in combination with docetaxel:
NSCLC subjects of any histology may be enrolled. Subjects with a histological or cytological diagnosis of non-squamous NSCLC must not have an EGFR-sensitizing mutation and/or ALK translocation/ROS1 rearrangement. EGFR sensitizing mutations are those mutations that are amenable to treatment with an approved TKI. Documentation of EGFR and ALK status should be available per local assessment. If documentation of EGFR and ALK status is unavailable, sponsor medical monitor approval is required prior to enrollment.
Subject has progressed during/after treatment with an anti-PD1/Ligand 1 (L1) monoclonal antibody (mAb) administered either as monotherapy, or in combination.
Subjects have progressed during/after platinum doublet chemotherapy for metastatic disease with or without an anti-PD1/Ligand 1 (L1) monoclonal antibody mAb.
   Disease progression during/after PD-anti-PD1/Ligand 1 (L1)1 treatment progression is defined by meeting the following criteria:
      Has received at least 2 doses of an approved anti-PD-1/L1 mAb
      Has demonstrated disease progression (PD) during/after anti-PD-1/L1 as defined by RECIST 1.1.

Preliminary Results:

By November 2021 a total of 12 patients had been enrolled and dosed with GEN1046 and Docetaxel: six patients had been dosed with 55 mg/m² Docetaxel and six patients had been dosed with 75 mg/m² Docetaxel. Four patients remained on treatment: one patient dosed with 55 mg/m² Docetaxel and three patients dosed with 75 mg/m² Docetaxel. A total of eight patients were off-treatment. The main reasons for off-treatment were: Documented radiographic disease progression (5 subjects), subject discontinued due to disease progression, per serious adverse event (SAE) report (1 subject), subject requested to discontinue trial treatment (1 subject) and discontinuation from respiratory failure due to disease progression (1 subject).

TABLE 15
Cumulative GEN1046 Treatment-Related adverse effects by
grade. A 55 mg/m² Docetaxel, B with 75 mg/m² Docetaxel.

| | | | | |
|---|---|---|---|---|
| Grade 1 | Grade 2 | Grade 3 | Grade 4 | Grade 5 |

A.

| | | | | | |
|---|---|---|---|---|---|
| Total treated subjects | 4 | 4 | 3 | 0 | 0 |
| with related TEAEs | | | | | |
| Anemia | 0 | 0 | 3 | 0 | 0 |
| Fatigue | 0 | 2 | 1 | 0 | 0 |
| Pyrexia | 1 | 0 | 0 | 0 | 0 |
| Nausea | 1 | 0 | 0 | 0 | 0 |
| Vaginal hemorrhage | 1 | 0 | 0 | 0 | 0 |
| Decreased appetite | 0 | 1 | 0 | 0 | 0 |
| Vomiting | 0 | 1 | 0 | 0 | 0 |
| Dyspnea | 0 | 1 | 0 | 0 | 0 |
| Blood alkaline | 0 | 1 | 0 | 0 | 0 |
| phosphatase increase | | | | | |
| Hypokalemia | 0 | 1 | 0 | 0 | 0 |
| Hypomagnesaemia | 1 | 0 | 0 | 0 | 0 |
| Sinus tachycardia | 0 | 1 | 0 | 0 | 0 |
| Alopecia | 1 | 0 | 0 | 0 | 0 |
| Neutrophil | 0 | 0 | 1 | 0 | 0 |
| count decreased | | | | | |

B

| | | | | | |
|---|---|---|---|---|---|
| Total treated subjects | 4 | 4 | 2 | 1 | 0 |
| with related TEAEs | | | | | |
| Fatigue | 1 | 2 | 0 | 0 | 0 |
| Dyspnea | 1 | 2 | 0 | 0 | 0 |
| Hyperglycemia | 1 | 0 | 1 | 0 | 0 |
| Alanine aminotransferase | 1 | 0 | 0 | 0 | 0 |
| increased | | | | | |
| Lipase increased | 0 | 1 | 0 | 0 | 0 |
| Nausea | 0 | 1 | 0 | 0 | 0 |
| Anemia | 0 | 1 | 0 | 0 | 0 |
| Vomiting | 1 | 0 | 0 | 0 | 0 |
| Amylase increased | 1 | 0 | 0 | 0 | 0 |
| Musculoskeletal | 0 | 1 | 0 | 0 | 0 |
| chest pain | | | | | |
| Cough | 1 | 0 | 0 | 0 | 0 |
| Pruritus | 1 | 0 | 0 | 0 | 0 |
| Dry mouth | 1 | 0 | 0 | 0 | 0 |
| Neutrophil | 0 | 0 | 0 | 1 | 0 |
| count decreased | | | | | |
| Gastroesophageal | 1 | 0 | 0 | 0 | 0 |
| reflux disease | | | | | |
| Blood thyroid stimulating | 1 | 0 | 0 | 0 | 0 |
| hormone abnormal | | | | | |
| Hiccups | 0 | 1 | 0 | 0 | 0 |
| Palmar plantar | 1 | 0 | 0 | 0 | 0 |
| erythrodysesthesia | | | | | |
| Syndrome (PPE) | | | | | |

TABLE 16
Docetaxel Treatment-Related adverse effects by grade.
A 55 mg/m² Docetaxel, B with 75 mg/m² Docetaxel.

| | | | | | |
|---|---|---|---|---|---|
| AE term (Coded) | Grade 1 | Grade 2 | Grade 3 | Grade 4 | Grade 5 |

A

| | | | | | |
|---|---|---|---|---|---|
| Anemia | 1 | 2 | 2 | 0 | 0 |
| Fatigue | 0 | 2 | 1 | 0 | 0 |
| Febrile neutropenia | 0 | 0 | 1 | 0 | 0 |
| Nausea | 1 | 0 | 0 | 0 | 0 |
| Dysgeusia | 0 | 1 | 0 | 0 | 0 |
| Neutrophil | 0 | 0 | 0 | 1 | 0 |
| count decreased | | | | | |
| White blood cell | 0 | 0 | 0 | 1 | 0 |
| count decreased | | | | | |
| Hypokalemia | 0 | 1 | 0 | 0 | 0 |
| Hypomagnesaemia | 1 | 0 | 0 | 0 | 0 |
| Pyrexia | 1 | 1 | 0 | 0 | 0 |
| Vomiting | 0 | 2 | 0 | 0 | 0 |
| Blood alkaline | 0 | 1 | 1 | 0 | 0 |
| phosphatase increase | | | | | |
| Hyponatremia | 1 | 0 | 0 | 0 | 0 |
| Infusion site | 1 | 0 | 0 | 0 | 0 |
| extravasation | | | | | |

B

| | | | | | |
|---|---|---|---|---|---|
| Fatigue | 1 | 2 | 0 | 0 | 0 |
| Nausea | 1 | 2 | 0 | 0 | 0 |
| Anaemia | 0 | 0 | 1 | 0 | 0 |
| Lacrimation increased | 1 | 0 | 0 | 0 | 0 |
| Diarrhea | 1 | 0 | 0 | 0 | 0 |
| Not coded: periphery | 1 | 0 | 0 | 0 | 0 |
| sensory neuropathy | | | | | |
| Dyspnea | 0 | 0 | 0 | 1 | 0 |
| Gastrooesophageal | 0 | 1 | 0 | 0 | 0 |
| reflux disease | | | | | |
| Hiccups | 0 | 1 | 0 | 0 | 0 |
| Vomiting | 1 | 0 | 0 | 0 | 0 |
| Neutrophil | 0 | 0 | 1 | 1 | 0 |
| count decreased | | | | | |

Toxicities of Special Interest:

Hepatotoxicity: No reported drug-related hepatoxicities to either GEN1046 or Docetaxel

Myelosuppression: No new adverse effects of neutropenia in the DLT period. No major difference in anemia between dose levels

Immune-related toxicities: None reported for GEN1046

There were no dose reductions or withdrawals in the DLT period. Docetaxel was interrupted during a single event of infusion site extravasation (55 mg/m²).

Efficacy:

Preliminary data on efficacy was obtained from the six subjects dosed with GEN1046 and 75 mg/m² Docetaxel. In one of the six patients the first post-baseline scan was indicative of response according to RECIST Criteria v1.1.

### Summary:

12 subjects were dosed
No Dose Limiting Toxicity reported at ether dose level of Docetaxel (55 mg/m² or 75 mg/m²)
No serious adverse events related to GEN1046 in the dose limiting toxicity period
The higher dose of 75 mg/m² Docetaxel is manageable.
No immune related toxicity, data do not suggest added or synergistic toxicity
The combination is manageable and well tolerated
Preliminary indication of response in subjects dosed with GEN1046 and 75 mg/m² Docetaxel.

1. A method for reducing or preventing progression of a tumor or treating cancer in a subject, comprising providing to the subject combined treatment with
I) a binding agent comprising a first binding region binding to human CD137, and a second binding region binding to human PD-L1; and
ii) a taxane chemotherapeutic agent.

2. The method according to claim 1, comprising administering the binding agent and the taxane chemotherapeutic agent to said subject in at least one treatment cycle.

3. The method according to any one of the preceding claims, wherein the first binding region binds to human CD137 having the sequence set forth in SEQ ID NO: 24, and/or the second binding region binds to human PD-L1 having the sequence set forth in SEQ ID NO: 26.

4. The method according to any one of the preceding claims, wherein the binding agent activates human CD137 when bound thereto and inhibits the binding of human PD-L1 to human PD-1 when bound to PD-L1.

5. The method according to any one of the preceding claims, wherein
a) the first binding region comprises a heavy chain variable region (VH) comprising the CDR1, CDR2, and CDR3 sequences of SEQ ID NO: 1, and a light chain variable region (VL) comprising the CDR1, CDR2, and CDR3 sequences of SEQ ID NO: 5;
and
b) the second antigen-binding region comprises a heavy chain variable region (VH) comprising the CDR1, CDR2, and CDR3 sequences of SEQ ID NO: 8, and a light chain variable region (VL) comprising the CDR1, CDR2, and CDR3 sequences of SEQ ID NO: 12.

6. The method according to any one of the preceding claims, wherein
a) the first binding region comprises a heavy chain variable region (VH) comprising the CDR1, CDR2, and CDR3 sequences set forth in: SEQ ID NO: 2, 3, and 4, respectively, and a light chain variable region (VL) comprising the CDR1, CDR2, and CDR3 sequences set forth in: SEQ ID NO: 6, GAS, 7, respectively;
and
b) the second antigen-binding region comprises a heavy chain variable region (VH) comprising the CDR1, CDR2, and CDR3 sequences set forth in: SEQ ID NO: 9, 10, 11 respectively, and a light chain variable region (VL) comprising the CDR1, CDR2, and CDR3 sequences set forth in: SEQ ID NO: 13, DDN, 14, respectively.

7. The method according to any one of the preceding claims, wherein
a) The first binding region comprises a heavy chain variable region (VH) comprising an amino acid sequence having at least 90%, at least 95%, at least 97%, at least 99%, or 100% sequence identity to SEQ ID NO: 1 and a light chain variable region (VL) region comprising an amino acid sequence having at least 90%, at least 95%, at least 97%, at least 99%, or 100% sequence identity to SEQ ID NO: 5;
and
b) the second binding region comprises a heavy chain variable region (VH) comprising an amino acid sequence having at least 90%, at least 95%, at least 97%, at least 99%, or 100% sequence identity to SEQ ID NO: 8 and a light chain variable region (VL) region comprising an amino acid sequence having at least 90%, at least 95%, at least 97%, at least 99%, or 100% sequence identity to SEQ ID NO: 12.

8. The method according to any one of the preceding claims, wherein
a) The first binding region comprises a heavy chain variable region (VH) comprising the amino acid sequence set forth in SEQ ID NO: 1 and a light chain variable region (VL) region comprising the amino acid sequence set forth in SEQ ID NO: 5;
and
b) the second binding region comprises a heavy chain variable region (VH) comprising the amino acid sequence set forth in SEQ ID NO: 8 and a light chain variable region (VL) region comprising the amino acid sequence set forth in SEQ ID NO: 12.

9. The method according to any one of the preceding claims, wherein the binding agent is an antibody, a multispecific antibody, such as a bispecific antibody.

10. The method according to any one of the preceding claims, wherein the binding agent is in the format of a full-length antibody or an antibody fragment.

11. The method according to any one of claims 5-10, wherein each variable region comprises three complementarity determining regions (CDR1, CDR2, and CDR3) and four framework regions (FR1, FR2, FR3, and FR4).

12. The method according to claim 11, wherein said complementarity determining regions and said framework regions are arranged from amino-terminus to carboxy-terminus in the following order: FR1, CDR1, FR2, CDR2, FR3, CDR3, FR4.

13. The method according to any one of the preceding claims, which comprises
i) a polypeptide comprising, consisting of or consisting essentially of, said first heavy chain variable region (VH) and a first heavy chain constant region (CH), and
ii) a polypeptide comprising, consisting of or consisting essentially of, said second heavy chain variable region (VH) and a second heavy chain constant region (CH).

14. The method according to any one of the preceding claims, which comprises
i) a polypeptide comprising said first light chain variable region (VL) and further comprising a first light chain constant region (CL), and
a polypeptide comprising said second light chain variable region (VL) and further comprising a second light chain constant region (CL).

15. The method according to any one of the preceding claims, wherein the binding agent is an antibody comprising a first binding arm and a second binding arm, wherein
the first binding arm comprises
i) a polypeptide comprising said first heavy chain variable region (VH) and said first heavy chain constant region (CH), and
ii) a polypeptide comprising said first light chain variable region (VL) and said first light chain constant region (CL);
and the second binding arm comprises
i) a polypeptide comprising said second heavy chain variable region (VH) and said second heavy chain constant region (CH), and
ii) a polypeptide comprising said second light chain variable region (VL) and said second light chain constant region (CL).

16. The method according to any one of the preceding claims, which comprises
i) a first heavy chain and light chain comprising said antigen-binding region capable of binding to CD137, and
a second heavy chain and light chain comprising said antigen-binding region capable of binding PD-L1.

17. The method according to any one of the preceding claims, wherein said binding agent comprises
i) a first heavy chain and light chain comprising said antigen-binding region capable of binding to CD137, the first heavy chain comprising a first heavy chain constant region and the first light chain comprising a first light chain constant region; and
ii) a second heavy chain and light chain comprising said antigen-binding region capable of binding PD-L1, the second heavy chain comprising a second heavy chain constant region and the second light chain comprising a second light chain constant region.

18. The method according to any one of claims 13-17, wherein each of the first and second heavy chain constant regions (CH) comprises one or more of a constant heavy chain 1 (CH1) region, a hinge region, a constant heavy chain 2 (CH2) region and a constant heavy chain 3 (CH3) region, preferably at least a hinge region, a CH2 region and a CH3 region.

19. The method according to any one of claims 13-18, wherein each of the first and second heavy chain constant regions (CHs) comprises a CH3 region and wherein the two CH3 regions comprise asymmetrical mutations.

20. The method according to any one of claims 13-19, wherein in said first heavy chain constant region (CH) at least one of the amino acids in a position corresponding to a position selected from the group consisting of T366, L368, K370, D399, F405, Y407, and K409 in a human IgG1 heavy chain according to EU numbering has been substituted, and in said second heavy chain constant region (CH) at least one of the amino acids in a position corresponding to a position selected from the group consisting of T366, L368, K370, D399, F405, Y407, and K409 in a human IgG1 heavy chain according to EU numbering has been substituted, and wherein said first and said second heavy chains are not substituted in the same positions.

21. The method according to claim 20, wherein (i) the amino acid in the position corresponding to F405 in a human IgG1 heavy chain according to EU numbering is L in said first heavy chain constant region (CH), and the amino acid in the position corresponding to K409 in a human IgG1 heavy chain according to EU numbering is R in said second heavy chain constant region (CH), or (ii) the amino acid in the position corresponding to K409 in a human IgG1 heavy chain according to EU numbering is R in said first heavy chain, and the amino acid in the position corresponding to F405 in a human IgG1 heavy chain according to EU numbering is L in said second heavy chain.

22. The method according to any of the preceding claims, wherein said binding agent induces Fc-mediated effector function to a lesser extent compared to another antibody comprising the same first and second antigen binding regions and two heavy chain constant regions (CHs) comprising human IgG1 hinge, CH2 and CH3 regions.

23. The method according to claim 22, wherein said first and second heavy chain constant regions (CHs) are modified so that the antibody induces Fc-mediated effector function to a lesser extent compared to an antibody which is identical except for comprising non-modified first and second heavy chain constant regions (CHs).

24. The method according to claim 23, wherein each of said non-modified first and second heavy chain constant regions (CHs) comprises the amino acid sequence set forth in SEQ ID NO: 15.

25. The method according to any of claims 23-24, wherein said Fc-mediated effector function is measured by binding to Fcγ receptors, binding to C1q, or induction of Fc-mediated cross-linking of Fcγ receptors.

26. The method according to claim 25, wherein said Fc-mediated effector function is measured by binding to C1q.

27. The method according to any one of claims 22-26, wherein said first and second heavy chain constant regions have been modified so that binding of C1q to said antibody is reduced compared to a wild-type antibody, preferably reduced by at least 70%, at least 80%, at least 90%, at least 95%, at least 97%, or 100%, wherein C1q binding is preferably determined by ELISA.

28. The method according to any one of the preceding claims, wherein in at least one of said first and second heavy chain constant regions (CH), one or more amino acids in the positions corresponding to positions L234, L235, D265, N297, and P331 in a human IgG1 heavy chain according to EU numbering, are not L, L, D, N, and P, respectively.

29. The method according to claim 28, wherein the positions corresponding to positions L234 and L235 in a human IgG1 heavy chain according to EU numbering are F and E, respectively, in said first and second heavy chains.

30. The method according to claim 28 or 29, wherein the positions corresponding to positions L234, L235, and D265 in a human IgG1 heavy chain according to EU numbering are F, E, and A, respectively, in said first and second heavy chain constant regions (HCs).

31. The method according to any one of claims 28-30, wherein the positions corresponding to positions L234 and L235 in a human IgG1 heavy chain according to EU numbering of both the first and second heavy chain constant regions are F and E, respectively, and wherein (i) the position corresponding to F405 in a human IgG1 heavy chain according to EU numbering of the first heavy chain constant region is L, and the position corresponding to K409 in a human IgG1 heavy chain according to EU numbering of the second heavy chain is R, or (ii) the position corresponding to K409 in a human IgG1 heavy chain according to EU numbering of the first heavy chain constant region is R, and the position corresponding to F405 in a human IgG1 heavy chain according to EU numbering of the second heavy chain is L.

32. The method according to any one of claims 28-31, wherein the positions corresponding to positions L234, L235, and D265 in a human IgG1 heavy chain according to EU numbering of both the first and second heavy chain constant regions are F, E, and A, respectively, and wherein (i) the position corresponding to F405 in a human IgG1 heavy chain according to EU numbering of the first heavy chain constant region is L, and the position corresponding to K409 in a human IgG1 heavy chain according to EU numbering of the second heavy chain constant region is R, or (ii) the position corresponding to K409 in a human IgG1 heavy chain according to EU numbering of the first heavy chain is R, and the position corresponding to F405 in a human IgG1 heavy chain according to EU numbering of the second heavy chain is L.

33. The method according to any one of claims 13-32, wherein the constant region of said first and/or second heavy chain comprises or consists essentially of or consists of an amino acid sequence selected from the group consisting of
a) the sequence set forth in SEQ ID NO: 15 [IgG1-FC],
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 10 substitutions, such as at the most 9 substitutions, at the most 8, at the most 7, at the most 6, at the most 5, at the most 4, at the most 3, at the most 2 or at the most 1 substitution compared to the amino acid sequence defined in a) or b).

34. The method according to any one of claims 13-33, wherein the constant region of said first or second heavy chain, such as the second heavy chain, comprises or consists essentially of or consists of an amino acid sequence selected from the group consisting of
a) the sequence set forth in SEQ ID NO: 16 [IgG1-F405L],
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 9 substitutions, such as at the most 8, at the most 7, at the most 6, at the most 5, at the most 4, at the most 3, at the most 2 or at the most 1 substitution compared to the amino acid sequence defined in a) or b).

35. The method according to any one of claims 13-34, wherein the constant region of said first or second heavy chain, such as the first heavy chain comprises or consists essentially of or consists of an amino acid sequence selected from the group consisting of
a) the sequence set forth in SEQ ID NO: 17 [IgG1-F409R]
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 10 substitutions, such as at the most 9 substitutions, at the most 8, at the most 7, at the most 6, at the most 5, at the most 4 substitutions, at the most 3, at the most 2 or at the most 1 substitution compared to the amino acid sequence defined in a) or b).

36. The method according to any one of claims 13-35, wherein the constant region of said first and/or second heavy chain comprises or consists essentially of or consists of an amino acid sequence selected from the group consisting of
a) the sequence set forth in SEQ ID NO: 18 [IgG1-Fc_FEA],
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 7 substitutions, such as at the most 6 substitutions, at the most 5, at the most 4, at the most 3, at the most 2 or at the most 1 substitution compared to the amino acid sequence defined in a) or b).

37. The method according to any one of claims 13-36, wherein the constant region of said first and/or second heavy chain, such as the second heavy chain, comprises or consists essentially of or consists of an amino acid sequence selected from the group consisting of
a) the sequence set forth in SEQ ID NO: 20 [IgG1-Fc_FEAL],
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 6 substitutions, such as at the most 5 substitutions, at the most 4 substitutions, at the most 3, at the most 2 or at the most 1 substitution compared to the amino acid sequence defined in a) or b).

38. The method according to any one of claims 13-37, wherein the constant region of said first and/or second heavy chain, such as the first heavy chain, comprises or consists essentially of or consists of an amino acid sequence selected from the group consisting of
a) the sequence set forth in SEQ ID NO: 19 [IgG1-Fc_FEAR]
b) a subsequence of the sequence in a), such as a subsequence wherein 1, 2, 3, 4, 5, 6, 7, 8, 9 or 10 consecutive amino acids has/have deleted, starting from the N-terminus or C-terminus of the sequence defined in a); and
c) a sequence having at the most 6 substitutions, such as at the most 5 substitutions, at the most 4, at the most 3, at the most 2 or at the most 1 substitution compared to the amino acid sequence defined in a) or b).

39. The method according to any one of the preceding claims, wherein said binding agent comprises a kappa (κ) light chain constant region.

40. The method according to any one of the preceding claims, wherein said binding agent comprises a lambda (λ) light chain constant region.

41. The method according to any one of the preceding claims, wherein said first light chain constant region is a kappa (κ) light chain constant region.

42. The method according to any one of the preceding claims, wherein said second light chain constant region is a lambda (λ) light chain constant region.

43. The method according to any one of the preceding claims, wherein said first light chain constant region is a lambda (λ) light chain constant region.

44. The method according to any one of the preceding claims, wherein said second light chain constant region is a kappa (κ) light chain constant region.

45. The method according to any one of claims 39-44, wherein the kappa (κ) light chain comprises an amino acid sequence selected from the group consisting of
a) the sequence set forth in SEQ ID NO: 21,
b) a subsequence of the sequence in a), such as a subsequence wherein 1, 2, 3, 4, 5, 6, 7, 8, 9 or 10 consecutive amino acids has/have been deleted, starting from the N-terminus or C-terminus of the sequence defined in a); and
c) a sequence having at the most 10 substitutions, such as at the most 9 substitutions, at the most 8, at the most 7, at the most 6, at the most 5, at the most 4 substitutions, at the most 3, at the most 2 or at the most 1 substitution, compared to the amino acid sequence defined in a) or b).

46. The method according to any one of claims 40-45, wherein the lambda (λ) light chain comprises an amino acid sequence selected from the group consisting of
a) the sequence set forth in SEQ ID NO: 22,
b) a subsequence of the sequence in a), such as a subsequence wherein 1, 2, 3, 4, 5, 6, 7, 8, 9 or 10 consecutive amino acids has/have been deleted, starting from the N-terminus or C-terminus of the sequence defined in a); and
c) a sequence having at the most 10 substitutions, such as at the most 9 substitutions, at the most 8, at the most 7, at the most 6, at the most 5, at the most 4 substitutions, at the most 3, at the most 2 or at the most 1 substitution, compared to the amino acid sequence defined in a) or b).

47. The method according to any one of the preceding claims, wherein the binding agent is of an isotype selected from the group consisting of IgG1, IgG2, IgG3, and IgG4.

48. The method according to any one of the preceding claims, wherein the binding agent is a full-length IgG1 antibody.

49. The method according to any one of the preceding claims, wherein said antibody is of the IgG1m(f) allotype.

50. The method according to any one of the preceding claims, wherein said antibody is acasunlimab or a biosimilar thereof.

51. The method according to any one of the preceding claims, wherein the taxane is selected from the group consisting of: Docetaxel, paclitaxel, cabazitaxel and tesetaxel.

52. The method according to any one of the preceding claims, wherein the taxane chemotherapeutic drug is docetaxel.

53. The method according to any one of the preceding claims, wherein the amount of binding agent administered in each dose and/or in each treatment cycle is
a) about 0.3-15 mg/kg body weight or about 25-1200 mg in total; and/or
b) about 2.1×10⁻⁹−1.2×10⁻⁷ mol/kg body weight or about 1.7×10⁻⁷−8.1×10⁻⁶ mol in total.

54. The method according to any one of the preceding claims, wherein the amount of binding agent administered in each dose and/or in each treatment cycle is
a) about 0.3-10 mg/kg body weight or about 25-800 mg in total; and/or
b) about 2.1×10⁻⁹−6.8×10⁻⁸ mol/kg body weight or about 1.7×10⁻²−5.4×10⁻⁶ mol in total.

55. The method according to any one of the preceding claims, wherein the amount of binding agent administered in each dose and/or in each treatment cycle is
a) about 0.3-5 mg/kg body weight or about 25-400 mg in total; and/or
b) about 2.1×10⁻⁹−3.4×10⁻⁸ mol/kg body weight or about 1.7×10⁻²−2.7×10⁻⁶ mol in total.

56. The method according to any one of the preceding claims, wherein the amount of binding agent administered in each dose and/or in each treatment cycle is
a) about 1.25 mg/kg body weight or about 100 mg in total; and/or
b) about 8.5×10⁻⁹ mol/kg body weight or about 6.8×10⁻² mol in total.

57. The method according to any one of the preceding claims, wherein the amount of taxane chemotherapeutic agent administered in each dose and/or in each treatment cycle is about 10-200 mg/m², such as 20-40 mg/m², 30-50 mg/m², 40-100 mg/m², 50-100 mg/m², 50-80 mg/m², 50-70 mg/m², 50-60 mg/m², 50-110 mg/m², 60-100 mg/m², 60-100 mg/m², 60-90 mg/m², 70-80 mg/m², 80-200 mg/m², 90-180 mg/m², 90-110 mg/m², 100-175 mg/m², or such as a bout 170-180 mg/m².

58. The method according to any one of the preceding claims, wherein the taxane chemotherapeutic agent is docetaxel and the amount administered in each dose and/or in each treatment cycle is about 50-60 mg/m², such as about 55 mg/m².

59. The method according to any one of the preceding claims, wherein the taxane chemotherapeutic agent is docetaxel and the amount administered in each dose and/or in each treatment cycle is about 70-80 mg/m², such as about 75 mg/m².

60. The method according to any one of the preceding claims, wherein the binding agent and/or the taxane chemotherapeutic agent is/are administered by systemic administration.

61. The method according to any one of the preceding claims, wherein the binding agent and/or the taxane chemotherapeutic agent is/are administered by intravenous injection or infusion.

62. The method according to any one of the preceding claims, wherein at least one dose of said binding agent and at least one dose of said taxana chemotherapeutic drug are administered in each treatment cycle.

63. The method according to any one of the preceding claims, wherein each treatment cycle is three weeks (21 days).

64. The method according to any one of the preceding claims, wherein said binding agent and said taxane chemotherapeutic agent are administered on the same day.

65. The method according to any one of the preceding claims, wherein each dose of said binding agent is infused over a minimum of 30 minutes, such as over a minimum of 60 minutes, a minimum of 90 minutes, a minimum of 120 minutes or a minimum of 240 minutes.

66. The method according to any one of the preceding claims, wherein administration of the binding agent precedes administration of the taxane chemotherapeutic agent by at least 30 minutes, such as by at least 1 hour or such as at least 2 hours.

67. The method according to any one of the preceding claims, wherein one dose of said binding agent and/or one dose of said taxane chemotherapeutic agent is/are administered on day 1 of each treatment cycle.

68. The method according to any one of the preceding claims, wherein one dose of said binding agent is administered every third week (1Q3W), such as on day 1 on each three-week treatment cycle.

69. The method according to any one of the preceding claims, wherein one dose of said taxane chemotherapeutic agent is administered every third week (1Q3W), such as on day 1 on each three-week treatment cycle.

70. The method according to any one of the preceding claims, wherein each dose of taxane chemotherapeutic agent is preceded by steroid premedication, such as premedication with an oral corticosteroid; e.g. administration of about 8 mg dexamethasone 2 times a day for 3 days starting 1 day prior to administration of the taxane chemotherapeutic agent.

71. The method according to any one of the preceding claims, wherein the subject is a human subject.

72. The method according to any one of the preceding claims, wherein the tumor or cancer is a solid tumor.

73. The method according to any one of the preceding claims, wherein the tumor or cancer is selected from the group consisting of melanoma, ovarian cancer, lung cancer (e.g. non-small cell lung cancer (NSCLC), colorectal cancer, head and neck cancer, gastric cancer, breast cancer, renal cancer, urothelial cancer, bladder cancer, esophageal cancer, pancreatic cancer, hepatic cancer, thymoma and thymic carcinoma, brain cancer, glioma, adrenocortical carcinoma, thyroid cancer, other skin cancers, sarcoma, multiple myeloma, leukemia, lymphoma, myelodysplastic syndromes, ovarian cancer, endometrial cancer, prostate cancer, penile cancer, cervical cancer, Hodgkin's lymphoma, non-Hodgkin's lymphoma, Merkel cell carcinoma and mesothelioma.

74. The method according to any one of the preceding claims, wherein the tumor or cancer is selected from the group consisting of lung cancer (e.g. non-small cell lung cancer (NSCLC), urothelial cancer (cancer of the bladder, ureter, urethra, or renal pelvis), endometrial cancer (EC), breast cancer (e.g. triple negative breast cancer (TNBC)), squamous cell carcinoma of the head and neck (SCCHN) (e.g. cancer of the oral cavity, pharynx or larynx) and cervical cancer.

75. The method according to any one of the preceding claims, wherein the tumor or cancer is a lung cancer.

76. The method according to claim 75, wherein the lung cancer is a non-small cell lung cancer (NSCLC), such as a squamous or non-squamous NSCLC.

77. The method according to claim 76, wherein the NSCLC does not have an epidermal growth factor (EGFR)-sensitizing mutation and/or anaplastic lymphoma (ALK) translocation/ROS1 rearrangement.

78. The method according to any one of claims 75-77, wherein the subject has received up to four prior systemic treatment regimens for advanced/metastatic disease and has experienced disease progression on or after last prior systemic treatment, such as disease progression determined by radiography.

79. The method according to claim 78, wherein the subject has received platinum-based chemotherapy.

80. The method according to claim 78, wherein the subject is not eligible for platinum-based therapy and has received alternative chemotherapy, e.g., a treatment with gemcitabine-containing regimen.

81. The method according to any one of the preceding claims, wherein the subject has received prior treatment with checkpoint inhibitor(s), such as agent(s) targeting PD-1/PD-L, such as a PD-1/PD-L1 inhibitor.

82. The method according to any one of the preceding claims, wherein the subject has experienced disease progression on or after treatment with checkpoint inhibitor(s), such as agent(s) targeting PD-1/PD-L, such as a PD-1/PD-L1 inhibitor.

83. The method according to any one of the preceding claims, wherein the subject has experienced disease progression on or after last prior treatment with checkpoint inhibitor(s), such as agent(s) targeting PD-1/PD-L, such as a PD-1/PD-L1 inhibitor.

84. The method according to any one of claims 78-83, wherein the subject has experienced disease progression on or after last prior systemic treatment, such as disease progression determined by radiography.

85. The method according to any one of the preceding claims, wherein the subject has not received prior treatment with checkpoint inhibitor(s), such as agent(s) targeting PD-1/PD-L, such as a PD-1/PD-L1 inhibitor.

86. The method according to any one of the preceding claims, wherein the tumor or cancer is an endometrial cancer.

87. The method according to claim 86, wherein the subject has epithelial endometrial histology including: endometrioid, serous, squamous, clear-cell carcinoma, or carcinosarcoma.

88. The method according to claim 86 or 87, wherein the subject has received up to four prior systemic treatment regimens for advanced/metastatic disease and has experienced disease progression on or after last prior systemic treatment, such as disease progression determined by radiography.

89. The method according to any one of claims 86-88, wherein the subject has not received prior treatment with checkpoint inhibitor(s), such as agent(s) targeting PD-1/PD-L, such as a PD-1/PD-L1 inhibitor.

90. The method according to any one of the preceding claims, wherein the tumor or cancer is an urothelial cancer, including cancer of the bladder, ureter, urethra, or renal pelvis.

91. The method according to claim 90, wherein the subject has received up to four prior systemic treatment regimens for advanced/metastatic disease and has experienced disease progression on or after last prior systemic treatment, such as disease progression determined by radiography.

92. The method according to claim 90 or 91, wherein the subject has received prior treatment with checkpoint inhibitor(s), such as agent(s) targeting PD-1/PD-L, such as a PD-1/PD-L1 inhibitor.

93. The method according to claim 90 or 91, wherein the subject has received platinum-based chemotherapy.

94. The method according to any one of claim 90 or 91, wherein the subject is not eligible for platinum-based therapy and has received alternative chemotherapy, e.g., a treatment with gemcitabine-containing regimen.

95. The method according to any one of the preceding claims, wherein the tumor or cancer is a breast cancer, such as a triple negative breast cancer (TNBC).

96. The method according to claim 95, wherein the TNBC is HER2 negative, such as determined by Fluorescence in situ hybridization (FISH) or determination of protein expression by immunohistochemistry. Progesterone receptor negative, estrogen receptor negative.

97. The method according to claim 95 or 96, wherein the subject has received at least one prior systemic treatment regimen for locally advanced/metastatic disease, such as at least one prior systemic treatment regimen including anthracycline-, taxane-, antimetabolite- or microtubule inhibitor-containing regimens.

98. The method according to claim 97, wherein the subject has received at the most 4 prior systemic treatment regimens for locally advanced/metastatic disease, such including as at least one prior systemic treatment regimen including anthracycline-, taxane-, antimetabolite- or microtubule inhibitor-containing regimens.

99. The method according to any one of claims 95-98, wherein the subject has received prior treatment with checkpoint inhibitor(s), such as agent(s) targeting PD-1/PD-L, such as a PD-1/PD-L1 inhibitor.

100. The method according to claim 99, wherein the subject has experienced disease progression on or after said prior treatment with checkpoint inhibitor(s), such as disease progression determined by radiography.

101. The method according to any one of claims 95-98, wherein the subject has not received prior treatment with checkpoint inhibitor(s), such as agent(s) targeting PD-1/PD-L, such as a PD-1/PD-L1 inhibitor.

102. The method according to any one of the preceding claims, wherein the tumor or cancer is a head and neck cancer, such as a squamous cell carcinoma of the head and neck (SCCHN).

103. The method according to claim 102, wherein the tumor or cancer is recurrent or metastatic SCCHN.

104. The method according to claim 102 or 103, wherein the tumor or cancer is cancer of the oral cavity, pharynx or larynx.

105. The method according to any one of claims 102-104, wherein the subject has received up to four prior systemic treatment regimens for recurrent/metastatic disease and has experienced disease progression on or after last prior systemic treatment, such as disease progression determined by radiography.

106. The method according to claim 105, wherein the subject has received platinum-based chemotherapy.

107. The method according to claim 105, wherein the subject is not eligible for platinum-based therapy and has alternative chemotherapy.

108. The method according to any one of claims 102-107, wherein the subject has received prior treatment with checkpoint inhibitor(s), such as agent(s) targeting PD-1/PD-L, such as a PD-1/PD-L1 inhibitor.

109. The method according to claim 108, wherein the subject has experienced disease progression on or after said prior treatment with checkpoint inhibitor(s), such as disease progression determined by radiography.

110. The method according to any one of claims 102-107, wherein the subject has not received prior treatment with checkpoint inhibitor(s), such as agent(s) targeting PD-1/PD-L, such as a PD-1/PD-L1 inhibitor.

111. The method according to any one of the preceding claims, wherein the tumor or cancer is a cervical cancer.

112. The method according to claim 111, wherein the cervical cancer is of squamous cell, adenocarcinoma or adenosquamous histology.

113. The method according to claim 111 or 112, wherein the subject has received at least one prior systemic treatment regimen for recurrent/metastatic disease, such as chemotherapy in combination with treatment targeting vascular endothelial growth factor A, such as treatment with bevacizumab, and has experienced disease progression on or after last prior systemic treatment, such as disease progression determined by radiography.

114. The method according to claim 113, wherein the subject has received at the most 4 prior systemic treatment regimens for recurrent/metastatic disease, including chemotherapy in combination with treatment targeting vascular endothelial growth factor A, such as treatment with bevacizumab.

115. The method according to any one of claims 111-114, wherein the subject has not received prior treatment with checkpoint inhibitor(s), such as agent(s) targeting PD-1/PD-L, such as a PD-1/PD-L1 inhibitor.

116. A composition comprising a taxane chemotherapeutic agent and a binding agent comprising a first binding region binding to human CD137, and a second binding region binding to human PD-L1.

117. The composition according to claim 116, wherein the amount of binding agent in the composition is between 25-400 mg or 1.7×10⁻⁷-2.7×10⁻⁶ mol.

118. The composition according to claim 116, comprising about 80 mg of said binding agent.

119. The composition according to any one of claims 116-118, wherein the binding agent is as defined in any one of claims 3-50.

120. The composition according to any one of claims 116-119, wherein the taxane chemotherapeutic drug is as defined in any one of claims 51-52.

121. The composition according to any one of claims 116-120, wherein the composition is for systemic administration.

122. The composition according to any one of claims 116-121, wherein the composition is for injection or infusion, such as intravenous injection or infusion.

123. The composition according to any one of claims 116-122, wherein the binding agent and the taxne chemotherapeutic drug are in aqueous solution, such in 0.9% NaCl (saline), at a volume of 50-500 mL, such as 100-250 mL.

124. A binding agent comprising a first binding region binding to human CD137, and a second binding region binding to human PD-L1 for use in treatment of cancer or for use in reducing or preventing progression of a tumor, wherein the binding agent is used in combination with a taxane chemotherapeutic agent.

125. The binding agent for use according to claim 124, the binding agent being as defined in any one of claims 3-50.

126. The binding agent for use according to claim 124 or 125, wherein the taxane chemotherapeutic agent is as defined in any one of claims 51-52.

127. A taxane chemotherapeutic agent for use in treatment of cancer or for use in reducing or preventing progression of a tumor, wherein the taxane chemotherapeutic agent is used in combination with a binding agent comprising a first binding region binding to human CD137, and a second binding region binding to human PD-11.

128. The taxane chemotherapeutic agent for use according to claim 127, wherein the binding agent is as defined in any one of claims 3-50.

129. The taxane chemotherapeutic agent for use according to claim 127 or 128, the taxane chemotherapeutic agent being as defined in any one of claims 51-52.


SEQUENCE LISTING (36 sequences)

SEQ ID NO: 1 - protein, 117 aa, Artificial sequence
  Antibody variable domain sequence
EVQLVESGGGLVQPGRSLRLSCTASGFSLNDYWMSWVRQAPGKGLEWVGYIDVGGSLYYAASVKGRFTISRDDSKSIAYLQMNSLKTEDTAVYYCARGGLTYGFDLWGQGTLVTVSS

SEQ ID NO: 2 - protein, 8 aa, Artificial sequence
  Antibody CDR sequence
GFSLNDYW

SEQ ID NO: 3 - protein, 7 aa, Artificial sequence
  Antibody CDR sequence
IDVGGSL

SEQ ID NO: 4 - protein, 11 aa, Artificial sequence
  Antibody CDR sequence
ARGGLTYGFDL

SEQ ID NO: 5 - protein, 110 aa, Artificial sequence
  Antibody variable domain sequence
DIVMTQSPSSLSASVGDRVTITCQASEDISSYLAWYQQKPGKAPKRLIYGASDLASGVPSRFSASGSGTDYTFTISSLQPEDIATYYCHYYATISGLGVAFGGGTKVEIK

SEQ ID NO: 6 - protein, 6 aa, Artificial sequence
  Antibody CDR sequence
EDISSY

SEQ ID NO: 7 - protein, 12 aa, Artificial sequence
  Antibody CDR sequence
HYYATISGLGVA

SEQ ID NO: 8 - protein, 121 aa, Artificial sequence
  Antibody variable domain sequence
EVQLLEPGGGLVQPGGSLRLSCEASGSTFSTYAMSWVRQAPGKGLEWVSGFSGSGGFTFYADSVRGRFTISRDSSKNTLFLQMSSLRAEDTAVYYCAIPARGYNYGSFQHWGQGTLVTVSS

SEQ ID NO: 9 - protein, 8 aa, Artificial sequence
  Antibody CDR sequence
GSTFSTYA

SEQ ID NO: 10 - protein, 8 aa, Artificial sequence
  Antibody CDR sequence
FSGSGGFT

SEQ ID NO: 11 - protein, 14 aa, Artificial sequence
  Antibody CDR sequence
AIPARGYNYGSFQH

SEQ ID NO: 12 - protein, 108 aa, Artificial sequence
  Antibody variable domain sequence
SYVLTQPPSVSVAPGQTARITCGGNNIGSKSVHWYQQKPGQAPVLVVYDDNDRPSGLPERFSGSNSGNTATLTISRVEAGDEADYYCQVWDSSSDHVVFGGGTKLTVL

SEQ ID NO: 13 - protein, 6 aa, Artificial sequence
  Antibody CDR sequence
NIGSKS

SEQ ID NO: 14 - protein, 11 aa, Artificial sequence
  Antibody CDR sequence
QVWDSSSDHVV

SEQ ID NO: 15 - protein, 329 aa, Homo Sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 329 aa, Artificial sequence
  Antibody constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 17 - protein, 329 aa, Artificial sequence
  Antibody constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 18 - protein, 329 aa, Artificial sequence
  Antibody constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 329 aa, Artificial sequence
  Antibody constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 20 - protein, 329 aa, Artificial sequence
  Antibody constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 21 - protein, 107 aa, Homo Sapiens [sequence omitted]

SEQ ID NO: 22 - protein, 106 aa, Homo Sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 23 - protein, 254 aa, Homo Sapiens
MGNSCYNIVALLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 24 - protein, 232 aa, Homo Sapiens
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 25 - protein, 289 aa, Homo Sapiens
MRIFAVFIFMTYWHLLNAFTVTVPKDLYVVEYGSNMTIECFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 26 - protein, 272 aa, Homo Sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 27 - protein, 1209 aa, Homo Sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 28 - protein, 114 aa, Artificial sequence
  Antibody variable domain sequence
QSLEESGGRLVTPGTPLTLTCTVSGFSLNDYWMSWVRQAPGKGLEWIGYIDVGGSLYYASWAKGRFTISRTSTTVDLKMTSLTTEDTATYFCARGGLTYGFDLWGPGTLVTVSS

SEQ ID NO: 29 - protein, 110 aa, Artificial sequence
  Antibody variable domain sequence
DIVMTQTPASVSEPVGGTVTINCQASEDISSYLAWYQQKPGQRPKRLIYGASDLASGVPSRFSASGSGTEYALTISDLESADAATYYCHYYATISGLGVAFGGGTEVVVK

SEQ ID NO: 30 - protein, 124 aa, Artificial sequence
  Antibody variable domain sequence
EMQLVESGGGLVQPGRSMKLSCAGSGFTLSDYGVAWVRQAPKKGLEWVAYISYAGGTTYYRESVKGRFTISRDNAKSTLYLQMDSLRSEDTATYYCTIDGYGGYSGSHWYFDFWGPGTMVTVSS

SEQ ID NO: 31 - protein, 106 aa, Artificial sequence
  Antibody variable domain sequence
DIQMTQSPSLLSASVGDRVTLNCRTSQNVYKNLAWYQQKLGEAPKLLIYNANSLQAGIPSRFSGSGSGTDFTLTISSLQPEDVATYFCQQYYSGNTFGAGTNLELK

SEQ ID NO: 32 - protein, 118 aa, Artificial sequence
  Antibody variable domain sequence
EVQLVESGGGLVQPGGSLRLSCAASGFTFSDSWIHWYRQAPGKGLEWYAWISPYGGSTYYADSVKGRFTISADTSKNTAYLQMNSLRAEDTAVYYCARRHWPGGFDYWGQGTLVTVSS

SEQ ID NO: 33 - protein, 107 aa, Artificial sequence [sequence omitted]

SEQ ID NO: 34 - protein, 330 aa, Artificial sequence
  Antibody constant region
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPMVTCVVVDVSEDDPDVQISWFVNNVEVLTAQTQTHREDYNSTLRVVSALPIQHQDWMSGKEFKCKVNNKALPAPIERTISKPKGSVRAPQVYVLPPPEEEMTKKQVTLTCMVTDFMPEDIYVEWTNNGKTELNYKNTEPVLDSDGSYLMYSKLTVEKKNWVERNSYSCSVVHEGLHNHHTTKSFSRTPGK

SEQ ID NO: 35 - protein, 330 aa, Artificial sequence
  Antibody constant region
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPMVTCVVVDVSEDDPDVQISWFVNNVEVLTAQTQTHREDYNSTLRVVSALPIQHQDWMSGKEFKCKVNNKALPAPIERTISKPKGSVRAPQVYVLPPPEEEMTKKQVTLTCMVKDFMPEDIYVEWTNNGKTELNYKNTEPVLDSDGSYFMYSRLRVEKKNWVERNSYSCSVVHEGLHNHHTTKSFSRTPGK

SEQ ID NO: 36 - protein, 107 aa, Mus musculus
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC